(19) United States
(12) Patent Application Publication
(10) Pub. No.: US 2026/0284263 A1
(43) Pub. Date: Sep. 24, 2026

(54) METHOD FOR FABRICATION AND MODULATION OF EXTRACELLULAR MATRIX BIOMATERIALS

(71) Applicant: The Chinese University of Hong Kong, Shatin, New Territories, Hong Kong SAR (CN)

(72) Inventors: Dan WANG, Taipo, New Territories, Hong Kong SAR (CN); Rocky Sung Chi TUAN, Shatin, New Territories, Hong Kong SAR (CN); Dai Fei Elmer KER, Taipo, New Territories, Hong Kong SAR (CN); Shu Ting HUANG, Shatin, New Territories, Hong Kong SAR (CN); Yang XU, Taipo, New Territories, Hong Kong SAR (CN)

(21) Appl. No.: 19/475,398

(22) PCT Filed: Apr. 19, 2024
(86) PCT No.: PCT/CN2024/088835

§ 371 Date: Oct. 14, 2025

Related U.S. Application Data
(60) Provisional application No. 63/460,860, filed on Apr. 20, 2023.

(51) Int. Cl. A61L 27/36; A61L 27/18; A61L 27/22; A61L 27/52; A61L 27/54

(52) U.S. Cl. CPC A61L 27/3633; A61L 27/18; A61L 27/222; A61L 27/52; A61L 27/54

(57) ABSTRACT

Provided herein are graft materials particularly suitable for use to repair tendon defects including massive rotator cuff tendon defects. The provided graft materials have a hybrid core-shell structure, where the shell includes a photocrosslinkable hydrogel and an extracellular matrix, and where the core includes an elastomeric polymer covalently crosslinked to the shell. Also provided are methods of producing the disclosed graft materials, and methods of using the graft materials to repair a tendon defect.

### CROSS-REFERENCES TO RELATED APPLICATIONS

The present application claims priority to U.S. Provisional Application No. 63/460,860, filed Apr. 20, 2023, the full disclosure of which is incorporated by reference in its entirety for all purposes.

### BACKGROUND

Tendons are anatomical structures that connect muscles to bones and generate force transmission during joint movement (C. Rieu, L. Picaut, G. Mosser & L. Trichet, Curr. Pharm. Des. 23, (2017): 3483; F. Wu, M. Nerlich & D. Docheva, EFORT Open Rev. 2, (2017): 332; D. Wang et al., Biomaterials 272, (2021): 120789). Despite its strong mechanical strength, tendon tears often occur because of acute (overload) or degenerative (aging) processes (R. A. Sun Han Chang, J. F. Shanley, M. E. Kersh & B. A. C. Harley, Sci Adv 6, (2020): eabb6763). Severe tendon injuries affect more than 102.5 million adults every year and place a considerable burden on healthcare systems (>$2 billion annually, with postsurgery complications resulting in nearly 1 million additional days of inpatient care each year) (M. A. Fernandez-Yague, et al., Adv. Mater. 33, (2021): e2008788). Moreover, as population age and life expectancy increase, tendon injuries are becoming more common. Unfortunately, the natural healing process of tendons is slow due to its hypocellular and hypovascular nature, and repaired tendon can be inferior in structure and function (C. Rieu, L. Picaut, G. Mosser & L. Trichet, Cuff. Pharm. Des. 23, (2017): 3483; X. Zhang, D. Wang, K. K. Mak, R. S. Tuan & D. F. E. Ker, Front. Physiol. 12, (2021): 691954). For example, repaired tendon after clinical intervention can still lead to 20-90% failure rates (A. C. Abraham et al., Sci. Transl. Med. 11, (2019): eaav4319) with poor functional outcomes such as largely reduced mechanical strength (Young's modulus can be reduced by 60% compared to the normal tendon) (W. L. Lim, L. L. Liau, M. H. Ng, S. R. Chowdhury & J. X. Law, J. X. Tissue Eng. Regen. Med. 16, (2019): 549). As a result, clinical outcomes remain unsatisfactory due to high failure rates, risk of injury recurrence, limited availability of donor tendon grafts, and unsatisfactory long-term functional recovery, especially in cases of large-to-massive tendon injuries (F. Wu, M. Nerlich & D. Docheva, EFORT Open Rev. 2, (2017): 332).

To enhance tendon regeneration, especially in large-to-massive tears, the current clinical practice has focused on two major areas—maximizing mechanical support and optimizing the biological environment (G. Depres-Tremblay et al., J. Shoulder Elbow Surg. 25, (2016): 2078). This includes innovating on surgical repair techniques (double row, acromioplasty, maximizing footprint coverage), sutures and fixation devices (knotless suture anchors, FIBERWIRE, suture tape), and biomaterials (Synthetic: X-Repair, SportsMesh/Artelon; Biologic: RESTORE, ZIMMER® Collagen Repair Patch/PERMACOL™, GRAFTJACKET®, Conexa) (G. M. Gartsman, Shoulder Arthroscopy (Second Edition), W.B. Saunders, (2009); J. D. Rees, A. M. Wilson & R. L. Wolman, Rheumatology (Oxford) 45, (2006): 508). However, no single approach has resulted in complete functional repair. For example, GRAFTJACKET®, a popular clinical graft, exhibits biological activity such as increased collagen expression (R. D. J. Smith et al., Eur. Cells Mater. 31, (2016): 107), but is often inferior in terms of mechanical properties (e.g., storage modulus and toughness) when compared to synthetic grafts such as polyurethane-based SportsMesh/Artelon (S. Chaudhury, C. Holland, M. S. Thompson, F. Vollrath & A. J. Carr, J. Shoulder Elbow Surg. 21, (2012): 1168). This mismatched mechanical property presumably contributes to high re-tear rates, especially in large-to-massive tendon injuries (D. Qu, C. Z. Mosher, M. K. Boushell & H. H. Lu, Ann. Biomed. Eng. 43, (2015): 697).

To augment tendon repair, a number of growth factors (GFs) that have been shown to be active in both tendon development and healing, such as platelet-derived growth factor-BB (PDGF-BB), transforming growth factor-β1 (TGF-β1), and growth and differentiation factor-5 (GDF-5) (B. Y. Zhang, P. Xu, Q. Luo & G. B. Song, World J. Stem Cells 13, (2021): 115; M. Govoni et al., Tissue Eng. Part A 23, (2017): 811), have also been applied. However, the application of GFs in clinical tendon repair raises concerns such as off-target tissue formation. For example, GDF-5 and TGF-β have been shown to not only promote tendon healing but also to play roles in chondrogenesis and osteogenesis (Z. Mahmoudi et al., Carbohydr. Polym. 229, (2020): 115551; H. Shimaoka et al., J. Biomed. Mater. Res. A 68. (2004): 168).

Compared to GFs, tissue-derived extracellular matrix (ECM) exhibits potent and tissue-specific regenerative properties (G. Yang et al., Biomaterials 34, (2013): 9295). The ECM is a composite of cell-secreted molecules that offers biochemical and structural support to cells, tissues, and organs (H. R. Screen, D. E. Berk, K. E. Kadler, F. Ramirez & M. F. Young, J. Orthop. Res. 33, (2015): 793). This complex three-dimensional (3D) network of interacting macromolecules occupies the space between cells and is principally responsible for both force transmission and tissue structure maintenance (Y. S. Kim, M. Majid, A. J. Melchiorri & A. G. Mikos, Bioeng. Transl. Med. 4, (2019): 83). The ECM is also unique in its tissue-specific bioactivity because each tissue or organ contains a unique ECM composition that contributes to tissue-specific structure and function (Y. S. Kim, M. Majid, A. J. Melchiorri & A. G. Mikos, Bioeng. Transl. Med. 4, (2019): 83; B. B. Rothrauff, G. Yang & R. S. Tuan, Stem Cell Res. Ther. 8, (2017): 133). ECM-based scaffolds have various advantages over synthetic scaffolds, as their native protein composition allows the maintenance of native integrin-binding sites (E. Bianchi et al., Pharmaceutics 13, (2021): 89). Tendon ECM-supplemented hydrogels were shown to be capable of inducing human adipose-derived stem cell (hASC) proliferation and production of tenogenesis-associated proteins (B. B. Rothrauff, G. Yang & R. S. Tuan, Stem Cell Res. Ther. 8, (2017): 133). The implantation of ECM-based scaffolds significantly increased the ultimate failure load in a rat Achilles tendon (AT) full-thickness defect model compared to that achieved with the saline control (M. T. Spang, K. L. Christman, Acta Biomater. 68, (2018): 1).

As a result, ECM-based biomaterials have been used in a wide range of applications in both preclinical and clinical settings, including a number of Food and Drug Administration (FDA)-approved ECM scaffolds, such as GRAFTJACKET®, ZIMMER® Collagen Repair Patch/PERMACOL™, and TissueMend that have been utilized for clinical tendon repair (Y. Jiang, R. Li, C. Han & L. Huang, Int. J. Mol. Med. 47, (2021): 463; S. J. Lee, Y. S. Choi & S. W. Cho, Adv. Exp. Med. Biol. 1064, (2018): 161). Furthermore, the past decade has witnessed substantial progress towards the next generation of ECM-based biomaterials, such as the development of various decellularization techniques that can better preserve the native ECM structure and composition (G. S. Hussey, J. L. Dziki & S. F. Badylak, Nat. Rev. Mater. 3, (2018): 159). Indeed, tendon tissues have been directly decellularized and used as a scaffold material to promote tendon regeneration (S. Xie et al., J. Orthop. Res. 37, (2019): 887). Nevertheless, the dense collagenous architecture can serve as a barrier for cell infiltration, resulting in localization of cells and their subsequent proliferation occurring at the surface of the construct (C. W. Cheng, L. D. Solorio & E. Alsberg, Biotechnol. Adv. 32, (2014): 462; K. Shimomura, B. B. Rothrauff & R. S. Tuan, Am. J. Sports Med. 45, (2017): 604). To overcome these limitations while retaining the tissue-specific bioactivity inherent in the ECM, decellularized tissues can be further solubilized with enzymatic or chaotropic agents, resulting in a solution form that can be readily combined with a diverse array of biomaterials (B. B. Rothrauff, G. Yang & R. S. Tuan, Stem Cell Res. Ther. 8, (2017): 133; B. B. Rothrauff et al., J. Tissue Eng. Regen. Med. 12, (2018): e159).

Batch-to-batch variations, related to variables such as donor sex and age, directly influence the bioactivity of ECM-based scaffolds (H. R. Screen, D. E. Berk, K. E. Kadler, F. Ramirez & M. F. Young, J. Orthop. Res. 33, (2015): 793) and represent a major concern for their clinical applications (J. Kim et al., Nat. Commun. 8, (2017): 842; T. J. McKee, G. Perlman, M. Morris & S. V. Komarova, Sci. Rep. 9, (2019): 10542). To minimize these variations, it is important to decipher the functional components of the tendon ECM in order to achieve reproducible and reliable bioactivity, as well as optimal clinical efficacy in ECM-based tissue engineering strategies. The tendon ECM is composed predominantly of collagens (~60-85% dry weight) and other non-collagenous matrix (NCM) components (~15-40% dry weight), including proteoglycans, glycosaminoglycans (GAGs), and glycoproteins (N. Taye, S. Z. Karoulias & D. Hubmacher, J. Orthop. Res. 38, (2020): 23). Collagen fibers, which align parallel to the long axis of the tendon, have a hierarchical fibrous architecture and contribute to the structural and mechanical properties of tendon tissue (R. Parenteau-Bareil, R. Gauvin & F. Berthod, Materials (Basel) 3, (2010): 1863). Collagens have been widely utilized as a biomaterial in tendon tissue engineering, such as oriented collagen fiber membranes, collagen hydrogels, and electrospun collagen scaffolds (R. Parenteau-Bareil, R. Gauvin & F. Berthod, Materials (Basel) 3, (2010): 1863). Interestingly, tendon NCM components have also been found to play important roles in tendon development and regeneration. For example, decorin and biglycan contribute to collagen structure maintenance, fiber realignment, and tendon mechanical properties (N. Taye, S. Z. Karoulias & D. Hubmacher, J. Orthop. Res. 38, (2020): 23). Hyaluronic acid (HA), a high-molecular-weight GAG that is enriched in tendon ECM, can stimulate progenitor cell proliferation and collagen deposition as well as improve tendon healing (N. Taye, S. Z. Karoulias & D. Hubmacher, J. Orthop. Res. 38, (2020): 23; J. I. Liang et al., J. Mater. Sci. Mater. Med. 25, (2014): 217; T. de Wit et al., J. Orthop. Res. 27, (2009): 408). Chondroitin sulfate (CS), another GAG, has also been demonstrated with positive effects on tendon regeneration (L. Lippiello, Evid. Based Complement. Alternat. Med. 4, (2007): 219; H. Yuan et al., Int. J. Biol. Macromol. 170, (2021): 248). These finding suggest that NCM plays an important role in ECM-mediated tendon regeneration. However, compared to collagen, the function of the NCM in tendon regeneration is less understood (N. Taye, S. Z. Karoulias & D. Hubmacher, J. Orthop. Res. 38, (2020): 23), and an in-depth analysis of the functional components in tendon ECM active in tendon development and repair is needed to facilitate the development of ECM-based tendon tissue engineering strategies.

An additional requirement for ECM grafts to serve as an effective load augmentation device for functional tendon repair is that they must have the mechanical, structural, and suture retention properties necessary to withstand the high in vivo tensile loads on the repair site (J. Cui et al. NPJ Regen. Med. 7, (2022): 26; H. Liu et al., Acta Biomater. 56, (2017): 129; 1. Calejo et al., Adv. Healthc Mater. 11, (2022): e2102863). However, the disparity between the structural and material properties of current ECM scaffolds and native tendons suggests that in their current configuration, they may not be capable of providing appreciable mechanical reinforcement to primary rotator cuff repairs (M. Beldjilali-Labro et al., Materials (Basel) 11, (2018): 1116). Therefore, mechanical augmentation of ECM-based scaffolds is a potentially promising approach to improve their clinical performance for tendon repair. A potential, synergistic effect may be expected by using natural ECM in combination with synthetic biomaterials. Examples of such efforts include embedding collagen-glycosaminoglycan scaffolds with 3D-printed synthetic acrylonitrile butadiene styrene polymers (L. C. Mozdzen, R. Rodgers, J. M. Banks, R. C. Bailey & B. A. C. Harley, Acta Biomaterialia 33, (2016): 25), as well as electrochemical alignment of collagen to produce mechanically strong ECM-based scaffolds (A. Islam et al., Clin. Biomech. 30, (2015): 669). Such approaches have attained ultimate stresses (0.9 MPa) (L. C. Mozdzen, R. Rodgers, J. M. Banks, R. C. Bailey & B. A. Harley, Acta Biomater. 33, (2016): 25) and ultimate loads (59.9 N) (A. Islam et al., Clin. Biomech. 30, (2015): 669) that approach those of native tendons (human supraspinatus tendon (SSPT); ultimate stresses: 11.9-22.1 MPa (T. Matsuhashi et al., Clin. Anat. 27, (2014): 702); Ultimate load: 652 N (A. Ratcliffe et al., Ann. Biomed. Eng. 43, (2015): 819)). Despite such commendable efforts, it is noteworthy that the tensile properties of tendons vary dramatically, and for the supraspinatus tendon, which is the most frequently torn rotator cuff tendon, much higher mechanical attributes are desired (Tensile strength: 4.1-22.1 MPa (T. Matsuhashi et al., Clin. Anat. 27, (2014): 702; E. Itoi et al. J. Orthop. Res. 13, (1995): 578); Ultimate load: 652 N (A. Ratcliffe et al., Ann. Biomed. Eng. 43, (2015): 819)). In addition, there are few reports of large animal studies utilizing massive tendon defects that achieved similar mechanical attributes as native intact tendon. Therefore, using ECM-based scaffolds for tendon repair, while promising for its strong regenerative property, remains a compromise. Specifically, inferior mechanical properties of these scaffolds can lead to a high re-tear rate.

Thus, graft materials having improved mechanical and chemical properties are needed to provide more effective and robust treatment options, e.g., options for treating damage to tendons. The present disclosure addresses these and other needs by providing compositions and methods related to graft materials having several beneficial advantages, particularly for use in repairing tendon defects.

### BRIEF SUMMARY

The present disclosure generally relates to an improved hybrid scaffold graft material construct, which reconciles the need for a mechanically competent material, while retaining robust tenogenic features that are required for repairing large tendon defects. The provided graft material includes two distinct portions in the form of a core and a shell. The core portion includes an elastomeric polymer which possesses human tendon-like biomechanical properties and exceptional suture retention. The shell portion includes an extracellular matrix and a hydrogel which is photocrosslinked with the core portion to form the hybrid construct (FIG. 1).

In one aspect, the disclosure is to a graft material including a shell and a core. The shell of the graft material includes a photocrosslinkable hydrogel and an extracellular matrix. The core of the graft material includes an elastomeric polymer covalently linked to the shell.

In another aspect, the disclosure is to a method of repairing a defect of a tendon of a subject. The method includes implanting a graft material as disclosed herein in the subject proximate to the defect of the tendon.

In another aspect, the disclosure is to a method for producing a graft material. The method includes providing an extracellular matrix. The method further includes forming a pre-hydrogel mixture including a photocrosslinkable polymer and the extracellular matrix. The method further includes providing an elastomeric polymer. The method further includes absorbing a photocrosslinking agent onto a surface of the elastomeric polymer, thereby yielding a treated elastomeric polymer. The method further includes applying the pre-hydrogel mixture to the treated elastomeric polymer, thereby creating a pre-graft material. The method further includes irradiating the pre-graft material with ultraviolet light for an exposure duration, thereby producing the graft material.

### BRIEF DESCRIPTION OF THE DRAWINGS

FIG. 1 presents a schematic illustration of a fabrication process for a hybrid scaffold graft material in accordance with a provided embodiment. The core portion (elastomeric polymer, as a mechanically robust scaffold component) of the hybrid scaffold is bonded to a shell portion (hydrogel, as tenogenic cues) using benzophenone and UV-mediated photocrosslinking.

FIG. 2 presents a flow diagram of quality assurance and control steps and in vivo studies described in the Examples.

FIG. 3 presents a gel image showing that urea-extracted tendon extracellular matrix (tECM) contains abundant low molecular weight proteins (<100 kDa) that are absent in commercially available collagen type I solution (Col1). The consistency of tECM extraction was confirmed by a nearly identical protein profile among independent extracts of tECM (batches 1, 2, and 3).

FIG. 4 presents a series of graphs showing significantly reduced dsDNA content in tECM compared to untreated tendon tissues (raw tendon) based on a dsDNA assay. The hydroxyproline and sGAG assays showed the consistency of collagen and sGAG contents among 3 independent batches of tECM (n=3, mean±SEM; ***p<0.001).

FIG. 5 presents a series of microscopy images showing that, when used as a cell culture supplement, the tECM (10% v/v with 2% fetal bovine serum (FBS)) resulted in enhanced production of tenogenesis-associated markers (tenascin-C(TNC), and COL1) and F-actin compared to 2% FBS (v/v) and commercially available Col1 (10% v/v with 2% FBS) as observed using immunofluorescence staining after 6 days of culture (n=3 isolates).

FIG. 6 presents a schematic illustration of an improved dehydration process for preparing the polyol of a graft material elastomeric polymer in accordance with a provided embodiment. FIG. 6 also presents a table of mechanical characterization data demonstrating that the resulting elastomeric polymer possesses human tendon-like tensile properties.

FIG. 7 presents a diagram of ex vivo studies of release, degradation, and swelling behaviors of gelatin methacrylol (GelMA) and tECM-GeIMA hydrogels.

FIG. 8 presents a pair of graphs plotting data from a release test for a tECM-GelMA hydrogel. The tECM-GelMA hydrogel initiated a burst release within the first 2 days, followed by a sustained release profile over a period of 15 days (n=3, mean±SEM).

FIG. 9 presents a graph plotting data from a degradation test for GeIMA and tECM-GelMA hydrogels. The tECM-Ge1MA hydrogels showed a faster degradation profile compared to the GelMA hydrogel alone (n=4, mean±SEM; *p<0.05, **p<0.01).

FIG. 10 presents a graph plotting data from a swelling test for GeIMA and tECM-GelMA hydrogels. The swelling behavior of the tECM-GelMA hydrogel and GelMA hydrogel were comparable (n=3, mean f SEM).

FIG. 11 presents a schematic illustration showing implementation of a 90°-peeling test in accordance with American Society for Testing and Materials (ASTM) standard D638-10 for assessing the interface bonding integrity of the provided hybrid scaffold graft materials. FIG. 11 also presents a representative peeling force (per width)-displacement graph, bar plots of average peeling force and peak load, and images of bonded (photocrosslinked) and nonbonded (non-photocrosslinked) hybrid scaffold (n=8; mean±SEM; *, p<0.05).

FIG. 12 presents representative scanning electron microscope (SEM) images of a cross-section of bonded and nonbonded hybrid scaffold graft materials. White arrows indicate the interface of the GelMA hydrogel and QHM elastomer (n=3).

FIG. 13 presents a schematic illustration and images of bonded hybrid scaffold graft materials for mouse subcutaneous implantation studies. FIG. 13 also presents images from a histological analysis of subcutaneously implanted bonded hybrid scaffold at 7- and 18-days post-implantation. Black arrows indicate the interface of the GelMA hydrogel and QHM elastomer (n=3 per timepoint).

FIG. 14 presents a schematic illustration of tensile sample dimensions and a photograph of a tensile test set up for assessing tensile properties of the provided hybrid scaffold graft materials. FIG. 14 also presents representative force-displacement graphs and bar plots of tensile properties of GelMA hydrogel, acellular dermal matrix (ADM), QHM elastomer, and HyS (n=8; mean±SEM; **, p<0.01; ***, p<0.001).

FIG. 15 presents a schematic illustration of suture retention sample dimensions and a photograph of a suture retention test set up for assessing tensile properties of the provided hybrid scaffold graft materials. FIG. 15 also presents representative images before (0 N) and during (25 N) suture retention testing and suture migration value of ADM, QHM, and HyS (n=9; mean±SEM; ***, p<0.001).

FIG. 16 presents a schematic illustration of in vitro bioactivity assays of the cyto-bioactivity and histo-biocompatibility of tECM-HyS.

FIG. 17 presents microscopy images showing viability (live/dead assay) of hASCs encapsulated in HyS or tECM-HyS (Q: QHM elastomer, H: hydrogel).

FIG. 18 presents a graph plotting results from the viability assay of FIG. 17 (n=3, isolates per time point per group; live; red: dead; mean SEM; ns: p, <0.05).

FIG. 19 presents a graph showing proliferation (dsDNA assay) of hASCs encapsulated in HyS or tECM-HyS (n=3, isolates per timepoint per group; mean SEM; *, p<0.05).

FIG. 20 presents a series of graphs showing gene expression and fluorescence staining of tenogenesis-associated markers (TNC, COL1, TNMD) and cytoskeleton (F-actin) of hASCs encapsulated in HyS or tECM-HyS (n=3, isolates per timepoint per group; mean±SEM; *, p<0.05, **, p<0.01).

FIG. 21 presents a series of microscopy images from the assays plotted in the graphs of FIG. 20.

FIG. 22 presents a schematic illustration of a mouse subcutaneous implantation study of in vivo histo-biocompatibility. FIG. 22 also presents images of H&E and immunohistochemical (IHC) staining of subcutaneously implanted HyS and tECM-HyS after 7 and 28 days of implantation. Black arrows indicate the cell infiltration on scaffold surface. White dashed lines indicate the scaffold surface (T: tissue, S: scaffold; n=3 mice per group per timepoint).

FIG. 23 presents a schematic illustration showing the experimental overview of an in vivo assessment of tendon healing and gait function for tECM-HyS mediated repair in a rat massive rotator cuff tendon defect model.

FIG. 24 presents a schematic illustration and representative macroscopic images of surgical procedures according to the experimental overview of FIG. 23.

FIG. 25 presents a series of graphs showing data from a gait analysis according to the experimental overview of FIG. 23. The gait analysis includes 6 parameters, i.e., contact area, print width, stride length, print intensity, swing duration, and LII, for each of the control, defect only, and tECM-HyS groups at preoperative 2 days and postoperative 14, 28, and 56 days. Data were calculated by the ratio of the right front paws (surgery side) and the left front paws (contralateral side). FIG. 25 also presents representative prints of the right and left front paws of each group. Similar gait performance was observed in tECM-HyS and intact control group, indicating recovered shoulder function in the tECM-HyS group at 28-days post-implantation (n=10; mean±SEM; *, p<0.05; **, p<0.01; ***, p<0.001).

FIG. 26 presents data from a section from the original footprints recorded from a healthy rat (running to the right) using the CatWalk system a rat gait analysis, and the set run criteria. The length of each bar (in Print view) represents the duration of the stance phase for that particular paw. The space between bars represents the duration of the swing phase. The run speed of 28.15±7.47 was selected for analysis.

FIG. 27 presents images and graphs showing the gross appearance as well as measurement of cross-sectional area (CSA) as well as length of supraspinatus tendon (SSPT) and muscle for a macroscopic analysis of rat rotator cuff tendon healing (M: muscle, T: tendon, B: bone; n=10; mean SEM; **p<0.01).

FIG. 28 presents images from H&E staining of rat SSPT of intact control, defect only, and tECM-HyS groups for a histological analysis of rat rotator cuff tendon healing (n=3; T: tendon, NT: neotissue. Black arrows indicate aligned and wavy extracellular fibers embedded with tendon-like cells).

FIG. 29 presents reconstructed images and graphs from a microcomputed tomography (micro-CT) analysis of the structure of the proximal humerus in rats. Although in this animal model, tendon defects were only made on rat SSPT, bone tunnels were created to secure the scaffolds using sutures. Therefore, micro-CT was performed to evaluate bone healing within the bone tunnels. Representative micro-CT images of the proximal humerus of the rats are shown. Arrows indicate the bone tunnels created in the surgery. No statistically significant difference of bone volume fraction (bone volume/total volume; BV/TV), bone mineral density (BMD) of TV, and BMD of BV was observed between defect and tECM-HyS groups, which were significantly lower than intact control group. No statistically significant difference of trabecular number (Tb·N), trabecular thickness (Tb·Th), and trabecular separation (Tb·Sp) was observed among 3 groups. No evidence of ectopic ossification was observed in any group (n=8; mean±SEM; *p<0.05, **p<0.01, ***p<0.001).

FIG. 30 presents a schematic illustration showing an experimental overview for in vivo histological assessment of rotator cuff tendon healing for tECM-HyS mediated repair in rabbit massive rotator cuff tendon defect model at 1 month and 3 months after surgery.

FIG. 31 presents a schematic illustration and representative macroscopic images of surgical procedures according to the experimental overview of FIG. 30. Arrows indicate tendon and implanted scaffolds.

FIG. 32 presents microscopy images and graphs showing representative gross tissue appearance, histological observation (H&E, PR: picrosirius red, and PL: polarized light), and semiquantitative analyses of rabbit SSPT for the intact control, HyS, and tECM-HyS mediated repair groups at 1 month and 3 month after surgery. Data showed enhanced collagen synthesis and better ECM organization in tECM-HyS group, which was similar to intact control group at 3 month after surgery, indicating that tECM-HyS enhanced rotator cuff tendon regeneration and biomechanical features in a rabbit massive tendon defect (>1 cm) model (n=3; mean f SEM; *, p<0.05; for panel C, (*) as different from the control group, (#) as different from the HyS group).

FIG. 33 presents a microscopy image showing region of interest (ROI) selection for histological analysis of rabbit rotator cuff tendon healing. 4 ROI were selected around the implant sites as shown.

FIG. 34 presents representative microscopy images of picrosirius red staining and quantification of collagen area in the histological analysis of FIG. 33 using polarized light microscopy.

FIG. 35 presents representative SEM images at low (8000×) and at high (30000×) magnification taken from intact control, HyS, and tECM-HyS group at 3 months after surgery in a rabbit massive rotator cuff tendon defect model. FIG. 35 also presents graphs with a histogram and semi-quantitative analysis showing the distribution of fiber size of each group (n=3, mean±SEM; *, p<0.05).

FIG. 36 presents graphs showing nanoscale elastic modulus maps derived from nanoindenter and quantitative analyses of the intact control, HyS, and tECM-HyS groups from the experiment of FIG. 35.

FIG. 37 presents an illustration and photographs of a representative tensile test set up for mechanical assessment of tECM-HyS mediated repair in a rabbit massive rotator cuff tendon defect model at 3 months after surgery.

FIG. 38 presents an illustration of the ROI for tensile testing and failure mode for each group in the mechanical assessment of FIG. 37.

FIG. 39 presents results from the mechanical assessment of FIGS. 37 and 38 in graphs showing representative load/displacement curve, ultimate load, and stiffness of rabbit SSPT for intact control, HyS, and tECM-HyS groups at 3 months after surgery as well as HyS-RCT group. HyS-RCT: HyS repaired rabbit cadaver tendon (n=7; mean i SEM; *, p<0.05; **, p<0.01; ***, p<0.001).

FIG. 40 presents an image of an SDS-PAGE gel of enzymatically digested tECM. The tECM was digested with pepsin, HYAL, and ChABC and protein composition was analyzed by SDS-PAGE with Coomassie brilliant blue staining. Pepsin digestion (P-tECM lane) removed most of the protein bands with molecular weights lower than that of the collagen a chains (*) and β dimeric forms (see comparison to corresponding bands in the commercial Coll lane). While treatment with HYAL (HYAL-tECM lane) and ChABC (ChABC-tECM lane) did not significantly change the protein electrophoretic profile compared to tECM, some increased band intensity was observed in the <50 kDa molecular weight range.

FIG. 41 presents a schematic diagram of the cell culture setup used to test the pro-tenogenesis effects of enzymatically digested tECM on hASCs. Different enzymatically digested tECM preparations were applied to hASC culture as a medium supplement.

FIG. 42 presents an image of a Western blot analysis of enzymatically digested tECM. The analysis demonstrated the presence of fibronectin (FMOD) and fibromodulin (FN) in tECM, HYAL-tECM, and ChABC-tECM, but not in P-tECM. Biglycan (BGN) was detected in HYAL-tECM and ChABC-tECM but not tECM or P-tECM.

FIG. 43 presents a pair of graphs plotting collagen and sGAG concentrations in enzymatically digested tECM. While the collagen content was similar in all tested samples, the sGAG content was significantly higher in tECM and P-tECM than in HYAL-tECM and ChABC-tECM.

FIG. 44 presents results of a growth factor (GF) array analysis revealing similar levels of FGF-2, EGF, IGF-1, and TGF-β3 in all tested groups of enzymatically digested tECM.

FIG. 45 presents representative microscopy images of fluorescence staining of the cultures of FIG. 41. After 6 days of culture, DAPI staining suggested decreased hASC proliferation in the P-tECM-treated group compared to the tECM-treated group. No significant differences were observed among the HYAL-tECM-, ChABC-tECM-, and tECM-treated groups. The P-tECM-treated group exhibited decreased staining intensities of TNC, COL1, and F-actin compared to those in the other groups but similar to those in the Coll- and 2% FBS-treated groups.

FIG. 46 presents graphs showing results from semi-quantitative analyses of the cultures of FIG. 41. These results showed that pepsin digestion compromised the proliferative and tenogenic activity of tECM, whereas other enzyme digestions did not significantly affect these activities (n=3 isolates; mean±SEM; *, p<0.05; **, p<0.01; ***, p<0.001).

FIG. 47 presents graphs plotting the expression of tenogenesis-associated genes in hASCs treated with P-tECM. On day 3, significantly higher tenogenic gene expression (i.e., SCX and MKX) was observed in the tECM-treated group compared to other groups. By day 6, all tenogenic genes were expressed at significantly higher levels in the tECM-treated group than other groups. Pepsin digestion of the tECM (P-tECM) significantly reduced tenogenic gene expression in comparison to the tECM-treated group, which was comparable to controls treated with 2% FBS- and Coll-treated groups. (n=3 isolates; mean±SEM; *, p<0.05; **; p<0.01; *** p<0.001).

FIG. 48 presents representative microscopy images of fluorescence staining showing tenogenic bioactivity of P-tECM supplemented with 10 ng/mL of tenogenesis-associated growth factors (GFs). Under 10 ng/mL GF supplementation, the cell proliferation and the staining intensity of tenogenic markers (TNC and COL1) showed no significant difference compared to P-tECM-treated group and were significantly lower than the tECM-treated group.

FIG. 49 presents graphs showing results from semi-quantitative analyses of the cultures of FIG. 48 (n=3 isolates; mean SEM; *, p<0.05, **, p<0.01, ***, p<0.001).

FIG. 50 presents graphs plotting qPCR analysis data from the cultures of FIG. 48, showing that the treatment of TGF-β1, TGF-β3, and the GF mixture, but not FGF-2 or IGF-1, induced comparable tenogenesis-associated gene (i.e., TNC, MKX, and SCX) expression with tECM group (n=3 isolates; mean SEM; *, p<0.05, **, p<0.01, ***, p<0.001).

FIG. 51 presents representative microscopy images of fluorescence staining showing tenogenic bioactivity of P-tECM supplemented with 50 ng/mL of tenogenesis-associated GFs. Under 50 ng/mL GF supplementation, the cell proliferation showed no significant difference compared to P-tECM-treated group and were significantly lower than the tECM-treated group. The staining intensity of TNC, but not COL1 or F-actin, was enhanced in the TGF-β1- and TGF-β-supplemented P-tECM-treated groups but still significantly lower than the tECM-treated group. The staining intensities of all tenogenic markers in the FGF-2- and IGF-1-supplemented P-tECM-treated groups were not comparable to those in the tECM-treated group.

FIG. 52 presents graphs showing results from semi-quantitative analyses of the cultures of FIG. 51 (n=3 isolates; mean±SEM; *, p<0.05, **, p<0.01, ***, p<0.001).

FIG. 53 presents graphs plotting qPCR analysis data from the cultures of FIG. 51, showed that similar to tECM group, the treatment of TGF-β1 and TGF-β3 induced the tenogenesis- and proliferation-associated gene expression (i.e., TNC, MKX, and SCX), other genes were not significantly higher than FGF-2, IGF-1, and GF mixture group (n=3 isolates; mean±SEM; *, p<0.05, **, p<0.01, ***, p<0.001).

FIG. 54 presents a schematic diagram of a gelation-based fractionation of tECM into CM and NCM fractions in accordance with a provided embodiment.

FIG. 55 presents images from SDS-PAGE analyses for evaluating fractionation outcomes among four different thermal incubation durations (1, 4, 6, and 24 hours). SDS-PAGE/Coomassie blue staining showed that collagens and non-collagens were separated only after gelation for 4 hours and longer.

FIG. 56 presents images from SDS-PAGE and WB analyses for evaluating fractionation outcomes. Coomassie blue staining showed prominent collagen bands (α1-, α2-, and β-chains) in the 1 mg/mL tECM, Col1, and CM groups (all loaded at 1 mg/mL), but not evident in the NCM group. WB analysis showed that immune-positive collagen-associated bands (α- and β-chains) were present in the tECM, Col1, and CM groups but not in the NCM group.

FIG. 57 presents graphs showing results from BCA and hydroxyproline assays to quantify total protein collagen, and sGAG concentrations in CM, NCM, and tECM. The protein content by BCA assay in the NCM fraction was 97.48% (0.85±0.07 mg/mL) and CM fraction was 9.92% (0.10±0.01 mg/mL) of that in tECM. (n=4 repeats; mean f SEM; ***, p<0.001)

FIG. 58 presents a table showing results from BCA and hydroxyproline assays to quantify total protein collagen, and sGAG concentrations in CM, NCM, and tECM. For collagen content by hydroxyproline assay, the NCM fraction was (0.02±0.01 mg/mL) 14.71% and CM was (0.09±0.01 mg/mL) 85.28% of that in tECM. These results confirmed the efficient fractionation of collagen and non-collagenous components of tECM.

FIG. 59 presents data from GFs array analysis of CM, NCM, and tECM, showing that tECM, CM, and NCM exhibited comparable GF content, such as FGF-2, EGF, IGF-1, and TGF-β3. n=2, technical replicates. These results confirmed the efficient fractionation of collagen and non-collagenous components of tECM.

FIG. 60 presents a schematic diagram of NCM and CM fractionation in accordance with a provided embodiment.

FIG. 61 presents representative DAPI, cytoskeletal and immunofluorescence staining images showing tenogenic bioactivity of the CM and NCM tECM fractions in 2D culture. The NCM-treated group also showed enhanced staining intensities of tenogenic markers (TNC, and COL1) and F-actin compared to those in the CM-treated and Coll-treated groups.

FIG. 62 presents graphs showing results from semi-quantitative analyses of the cultures of FIG. 61. DAPI cell counting suggested that cell proliferation was higher in the tECM- and NCM-treated groups compared to the CM- and Coll-treated groups (n=3 isolates; mean±SEM; *, p<0.05; **, p<0.01; ***, p<0.001).

FIG. 63 presents graphs showing results from qPCR analyses for tenogenic markers in the cultures of FIG. 61. The NCM-treated group showed comparable gene expression of tenogenic markers (SCX, MKX, and TNC) with tECM-treated groups, which was significantly higher than those in the CM- and Col1-treated groups. Results indicated that the NCM fraction, but not the CM fraction, stimulated cell proliferation and tenogenic differentiation of hASCs to similar extents as tECM (n=3 isolates; mean SEM; *, p<0.05; **, p<0.01; ***, p<0.001).

FIG. 64 presents a schematic diagram of a cell culture setup used to test the tenogenic bioactivity of P-tECM supplemented with NCM.

FIG. 65 presents representative images of fluorescence staining for the cell culture of FIG. 58. Similar to tECM-treated group, both NCM- and P-tECM+NCM-treated group showed significantly enhanced staining intensities of tenogenic markers (TNC and COL1) and F-actin compared to those in the 2% FBS- and P-tECM-treated groups.

FIG. 66 presents graphs showing results from semiquantitative analyses for the cell culture of FIG. 64. DAPI cell counting suggested that cell proliferation was higher in the tECM-, NCM-, and P-tECM+NCM-treated groups compared to the 2% FBS- and P-tECM-treated groups. Results indicated that the supplementation of NCM fraction restored the lost bioactivity of acid-pepsin digested tECM, in terms of cell proliferation and tenogenic differentiation (n=3; mean±SEM; *, p<0.05; **, p<0.01; ***, p<0.001).

FIG. 67 presents a series of graphs showing results from qPCR analysis of the cell culture of FIG. 64 for tenogenesis- and proliferation-associated genes. The expression levels of tenogenesis-associated genes, including COL1A1, TNC, and COL3A1, were significantly higher in the tECM group compared to the control and P-tECM groups. Addition of NCM, i.e., NCM+P-tECM treatment of the control and P-tECM groups, significantly increased BGN expression (n=3, biological replicates; mean±SEM; *, p<0.05; **, p<0.01; ***, p<0.001).

FIG. 68 presents a schematic illustration of the experimental scheme to perform RNA-Seq analysis of tECM-, CM-, and NCM-driven hASC differentiation.

FIG. 69 presents a principal component analysis (PCA) graph for the experiment of FIG. 68, revealing the variance among the groups and indicating that a primary source of variation in transcriptional profiles is tied to groups with or without NCM.

FIG. 70 presents a heatmap of transcriptome analysis for the experiment of FIG. 68, showing data for 544 differentially expressed genes (DEGs) from three culture groups.

FIG. 71 presents a Venn diagram showing numbers of DEGs in three pairwise comparisons for the experiment of FIG. 68. These results suggest that compared to CM treatment, tECM and NCM treatment triggered similar transcriptional profiles. Also shown are volcano plots of upregulated and downregulated DEGs in the tECM versus CM and NCM versus CM groups.

FIG. 72 presents a heatmap of cell-specific gene signatures associated with stem cells, proliferation, tenogenesis, chondrogenesis, and osteogenesis across the three culture groups of the experiment of FIG. 68. The results show that both the tECM and NCM groups induce elevated expression of proliferation and tenogenesis genes compared with the CM group.

FIG. 73 presents data from a gene ontology (GO) enrichment analysis of DEGs in the tECM versus. CM and NCM versus CM groups of the experiment of FIG. 68. The size and color of the bubbles represent the fold enrichment and FDR value (negative log 10 transformed), respectively, of DEGs enriched in each term.

FIG. 74 presents data from a KEGG pathway analysis of DEGs in the tECM versus. CM and NCM versus CM groups of the experiment of FIG. 68. The size and color of the bubbles represent the fold enrichment and FDR value (negative log 10 transformed), respectively, of DEGs enriched in each pathway.

FIG. 75 presents data from a gene set enrichment analysis (GSEA) for the experiment of FIG. 68. A positive normalized enrichment score (NES) indicates enrichment in the tECM and NCM compared to CM groups. Presented NES are significant (FDR <0.25).

FIG. 76 presents a Venn diagram showing 118 overlapping genes that were upregulated in the tECM versus. CM and NCM versus CM groups of the experiment of FIG. 68.

FIG. 77 presents a series of hierarchical trees showing the biological process and cellular compounds GO enriched for the 118 overlapping genes of FIG. 76. The color of the nodes represents the significance of the corresponding GO terms. The results demonstrated that tECM and NCM shared similar GO enrichment patterns related to collagen synthesis; as well as demonstrated enrichment in gene sets associated with proliferation and tenogenesis (n=3, biological replicates).

FIG. 78 presents a schematic diagram of a cell culture setup used to test tenogenic bioactivities of the CM and NCM fractions of tECM in 3D hASC culture.

FIG. 79 presents representative images of fluorescence staining of tenogenic markers and F-actin. Results showed that NCM (both 1 and 3 mg/mL) showed comparable staining intensity of TNC and SCX with the tECM-treated group, which was higher than those in CM-, Coll-treated, and control groups.

FIG. 80 presents graphs showing results from qPCR analyses for tenogenic markers and proliferation-associated markers. The qPCR results showed that the tenogenic genes, i.e., SCX, MKX, and COL1A1, were expressed at comparable levels in the tECM- and NCM (1- or 3-mg/mL)-treated groups. The 3-mg/mL NCM group showed significantly enhanced expression levels of TNC over the 1-mg/mL NCM group, and enhanced levels of SCX, MKX and TNC over control, Col1-, and CM-treated groups (n=3 isolates; mean±SEM; *, p<0.05; **, p<0.01; ***, p<0.001).

FIG. 81 presents a schematic illustration and photographs of NCM and tECM concentration and centrifugation. Protein precipitation was observed in the 5-mg/mL tECM group but not in the 5-mg/mL NCM group, suggesting that NCM had better solubility than tECM under high concentration (n=3).

FIG. 82 presents a series of photographs and a graph showing that after 6 days of culture, hydrogels supplemented with tECM or NCM (1 mg/mL and 3 mg/mL) exhibited notable shrinkage compared to the other groups.

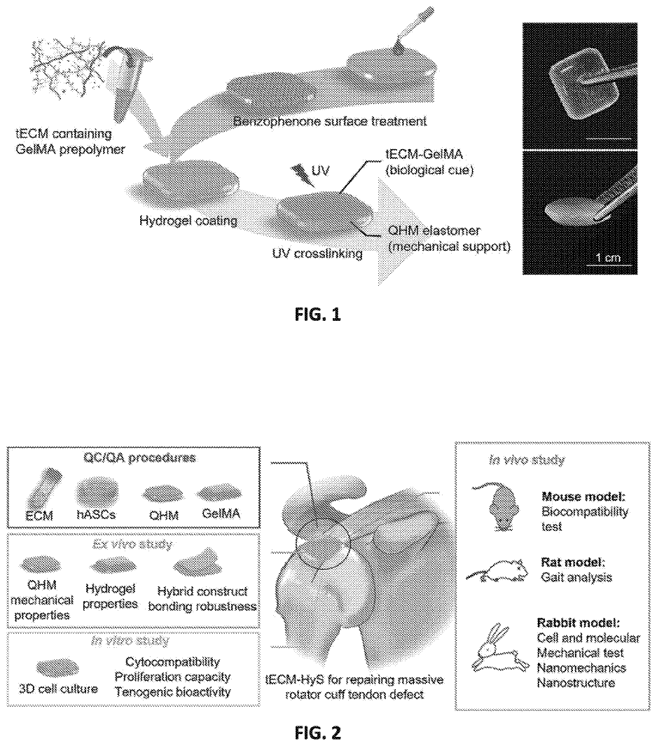

### DETAILED DESCRIPTION

### I. General

The present disclosure provides an extracellular matrix (ECM) composition that is engineered to include a composition of collagenous matrix-enriched and non-collagenous matrix (NCM)-enriched fractions. As demonstrated herein, NCM proteins can be more responsible than collagens for ECM pro-tenogenesis bioactivity desirable when, for example, using the ECM for tendon repair. For instance, the non-collagenous portion of the provided engineered ECM exhibits advantageous induces human adipose-derived stem cell (hASC) transcription of genes associated with cell proliferation, collagen synthesis, and tenogenic differentiation. Additionally, the ratio of collagens and non-collagenous matrix can be adjusted to enhance their bioactivity. Moreover, the enhanced solubility of the NCM-enriched fraction facilitates its combination with various water-soluble biomaterials for tissue engineering protocols.

The present disclosure also provides a core-shell structured, extracellular-matrix-containing hybrid scaffold (ECM-HyS) graft material including a hydrogel shell photocrosslinked to a mechanically robust elastomeric polymer core. The hydrogel and extracellular matrix of the shell advantageously provide the graft material with biological cues, while the elastomeric polymer core provides the graft material with beneficial load-bearing properties. The provided design elicits robust tendon regeneration and supports functional joint movement, both of which are extremely important for treating and repairing, for example, large-to-massive tendon injuries.

Because of the certain components of the provided graft material and their particular relative amounts, the graft material exhibits highly desirable mechanical characteristics suitable for applications such as tendon repair. For example, the graft materials disclosed herein exhibit biomechanical properties similar to those of the human supraspinatus tendon (SSPT), which has a tensile modulus of 462.78 MPa and a stiffness of 58.93 N/mm. The high mechanical strength of the graft material results at least in part from robust interfacial bonding between its hydrogel shell and elastomer core. Also advantageously, the provided graft material enables sustained release of extracellular matrix from its hydrogel shell. The graft material induces strong tendon differentiation in (hASCs and augments fast shoulder functional recovery within one month after implantation, as evidenced by gait analysis in a rat massive rotator cuff tendon defect model. The graft material also induces over 1 cm tendon-like tissue regeneration with robust biomechanical strength, as evidenced by histological analyses and mechanical tests in a rabbit massive rotator cuff tendon defect model. Another advantage of the provided graft material is its optional inclusion of non-collagenous matrix (NCM) contents of the extracellular matrix. As demonstrated herein, these non-collagenous extracellular matrix components can improve the pro-tenogenic bioactivity of the graft material in stimulating hASC proliferation and enhancing tenogenic differentiation.

### II. Definitions

As used herein, the term “photocrosslinkable” refers to a material that comprises one or more functional groups that may undergo a photochemical reaction to form covalent bonds with other functional groups on the same or different molecules upon exposure to a suitable light source, e.g., UV radiation or visible light. A photocrosslinkable material may be a polymer, a monomer, an oligomer, a macromolecule, or any combination thereof. A photocrosslinkable material may be natural or synthetic and may have various physical and chemical properties depending on its composition and structure. A photocrosslinkable material may be used to form hydrogels, coatings, adhesives, films, or other products that have desirable characteristics such as biocompatibility, biodegradability, mechanical strength and tunability.

As used herein, the term “hydrogel” refers to a highly-interdependent, biphasic matrix consisting of a solid component (usually a polymer, and more commonly a highly cross-linked polymer) that has both hydrophilic and hydrophobic character, and a liquid dispersion medium (e.g., water) that is retained in the matrix by intermolecular forces. The hydrophobic character provides the matrix with a degree of water insolubility while the hydrophilic character affords water permeability. One of skill in the art will appreciate that several different types of polymers may be used in combination to form hydrogels useful in the methods of the present invention. Being polymer networks that have high water-absorbing capacity, hydrogels often closely mimic native extracellular matrices. Hydrogels also tend to possess a degree of flexibility very similar to natural tissues, due to the relatively high water content. In some cases, hydrogels may contain well over 90% water.

As used herein, the term “extracellular matrix” refers to a composition of, e.g., macromolecules and minerals, that provides structural and biochemical support to surrounding cells. An extracellular matrix may be isolated from animal tissues, such as bone, skin, tendons, and cartilage. An extracellular matrix may alternatively be obtained from cultures of cells that secrete an extracellular matrix which can then be collected and purified.

As used herein, the term “polymer” refers to an organic substance composed of a plurality of repeating structural units (monomeric units) covalently linked to one another.

As used herein, the term “elastomeric polymer” refers to refers to a polymer that exhibits viscoelasticity (i.e., both viscosity and elasticity) and has weak intermolecular forces, low Young's modulus (E) and high failure strain compared with other materials. An elastomeric polymer may be composed of long chain-like molecules that can reconfigure themselves to distribute an applied stress and return to their original shape when the stress is removed. An elastomeric polymer may be natural or synthetic and may have various chemical structures and properties depending on its monomers and cross-linking agents.

As used herein, the term “growth factor” refers to a substance that promotes growth and/or cellular differentiation of specific tissues or cells. A growth factor may be a protein, a peptide, a hormone, a cytokine, or any other molecule that can bind to a receptor on a target cell and activate a signaling pathway that regulates cell proliferation, survival, migration, or differentiation. A growth factor may be produced by various types of tissues or cells, and may act in an autocrine, paracrine, endocrine, or juxtacrine manner. A “tenogenic growth factor” is a substance that promotes tenogenesis, which is the differentiation of cells into tenocytes or tendon-like cells. A tenogenic growth factor may belong to different families of growth factors, such as transforming growth factors beta (TGF-β), bone morphogenetic proteins (BMPs), fibroblast growth factors (FGFs), vascular endothelial growth factor (VEGF), connective tissue growth factor (CTGF), platelet-derived growth factor (PDGF), insulin-like growth factor 1 (IGF-1), or growth differentiation factor 7 (GDF-7). The tenogenic growth factor may act on various types of cells, such as mesenchymal stem cells (MSCs) or tendon progenitor cells, and may modulate their proliferation, survival, migration, or extracellular matrix production.

As used herein, the term “subject” refers to a vertebrate, and preferably to a mammal. Mammalian subjects for which the provided composition is suitable include, but are not limited to, mice, rats, simians, humans, farm animals, sport animals, and pets. In some embodiments, the subject is human. In some embodiments, the subject is male. In some embodiments, the subject is female. In some embodiments, the subject is an adult. In some embodiments, the subject is an adolescent. In some embodiments, the subject is a child. In some embodiments, the subject is above 10 years of age, e.g., above 20 years of age, above 30 years of age, above 40 years of age, above 50 years of age, above 60 years of age, above 70 years of age, or above 80 years of age. In some embodiments, the subject is less than 80 years of age, e.g., less than 70 years of age, less than 60 years of age, less than 50 years of age, less than 40 years of age, less than 30 years of age, less than 20 years of age, or less than 10 years of age.

As used herein, the terms “including,” “comprising,” “having,” “containing,” and variations thereof, are inclusive and open-ended and do not exclude additional, unrecited elements or method steps beyond those explicitly recited. As used herein, the phrase “consisting of” is closed and excludes any element, step, or ingredient not explicitly specified. As used herein, the phrase “consisting essentially of” limits the scope of the described feature to the specified materials or steps and those that do not materially affect the basic and novel characteristics of the disclosed feature.

As used herein, the singular forms “a,” “an,” and “the” include plural referents unless the content clearly dictates otherwise. Thus, for example, reference to “a polymer” optionally includes a combination of two or more polymers, and the like.

As used herein, the term “about” denotes a range of value that is +/−10% of a specified value. For instance, “about 10” denotes the value range of 9 to 11 (10+/−1).

### III. Graft Materials

In one aspect, the present disclosure provides various graft materials that generally include a shell with a photocrosslinkable hydrogel and an extracellular matrix (ECM), and a core with an elastomeric polymer covalently crosslinked to the shell. In some embodiments, the provided graft material is a hybrid scaffold (HyS) that includes a tendon extracellular matrix (tECM). The tendon hybrid construct, tECM-HyS, can be particularly useful for the repair of large-to-massive tendon defects. The hybrid construct design jointly harnesses the regenerative potential of a highly bioactive ECM-enriched hydrogel and a mechanically robust elastomer. Beneficially, provided graft materials demonstrate robust interfacial bonding to ensure scaffold integrity. The graft materials also exhibit excellent mechanical properties, e.g., human tendon-like tensile properties, and excellent suture retention that favors surgical repair and tendon functional movement. Additionally, the graft material can deliver bioactive agents in a sustained manner to, for example, support lengthy tendon repair processes. In vitro and preclinical evaluation demonstrated that the provided graft materials are biocompatibile, and that tECM-HyS embodiments augment shoulder functional movement, and achieve over 1 cm tendon regeneration with robust mechanical properties.

One advantageously improved property of the provided graft materials is their tensile modulus. The tensile modulus of a graft material determines the stiffness and resistance to deformation of the material when it is subjected to tensile loads. The tensile modulus of a graft material is important for repairing tendon defects because it relates to the biomechanical compatibility of the graft material and its ability to provide functional restoration of tendon defects. The tensile modulus of a graft material preferably should match or exceed that of the native tendon to avoid mechanical mismatch and failure of the repair. A graft material with a low tensile modulus may stretch excessively and compromise joint stability, while a graft material with a high tensile modulus may cause stress concentration and damage to the surrounding tissues.

The tensile modulus of the provided graft material can be, for example, between about 100 MPa and about 1000 MPa, e.g., between about 100 MPa and about 400 MPa, between about 130 MPa and about 500 MPa, between about 160 MPa and about 630 MPa, between about 200 MPa and about 790 MPa, or between about 250 MPa and about 1000 MPa. In terms of upper limits, the graft material tensile modulus can be, for example, less than about 1000 MPa, e.g., less than about 790 MPa, less than about 630 MPa, less than about 500 MPa, less than about 400 MPa, less than about 320 MPa, less than about 250 MPa, less than about 200 MPa, less than about 160 MPa, or less than about 130 MPa. In terms of lower limits, the graft material tensile modulus can be, for example, greater than 100 MPa, e.g., greater than about 130 MPa, greater than about 160 MPa, greater than about 200 MPa, greater than about 250 MPa, greater than about 320 MPa, greater than about 400 MPa, greater than about 500 MPa, greater than about 630 MPa, or greater than about 790 MPa. Higher tensile moduli, e.g., greater than about 1000 MPa, and lower tensile moduli, e.g., less than 100 MPa, are also contemplated. The tensile modulus can be measured according to, for example, the standard procedure of ASTM D638-10 (2010).

Another advantageously improved property of the provided graft materials is their stiffness, which can affect the mechanical behavior of a repaired tendon and ultimately influence the healing and functional outcomes. A graft material with a similar stiffness to the native tendon can help to distribute loads more evenly between the repaired tendon and the graft material. In contrast, a graft material that is too soft or too stiff may result in stress concentrations or stress shielding, which can cause the repaired tendon to be overloaded or underloaded. Moreover, the stiffness of the graft material can also influence the rate of tissue remodeling and the formation of new collagen fibers, which are important for the long-term strength and function of the repaired tendon.

The stiffness of the provided graft material can be, for example, between about 25 N/mm and about 85 N/mm, e.g., between about 25 N/mm and about 61 N/mm, between about 31 N/mm and about 67 N/mm, between about 37 N/mm and about 73 N/mm, between about 43 N/mm and about 79 N/mm, or between about 49 N/mm and about 85 N/mm. In terms of lower limits, the graft material stiffness can be, for example, less than about 85 N/mm, e.g., less than about 79 N/mm, less than about 73 N/mm, less than about 67 N/mm, less than about 61 N/mm, less than about 55 N/mm, less than about 49 N/mm, less than about 43 N/mm, less than about 37 N/mm, or less than about 31 N/mm. In terms of lower limits, the graft material stiffness can be, for example, greater than about 25 N/mm, e.g., greater than about 31 N/mm, greater than about 37 N/mm, greater than about 43 N/mm, greater than about 49 N/mm, greater than about 55 N/mm, greater than about 61 N/mm, greater than about 67 N/mm, greater than about 73 N/mm, or greater than about 79 N/mm. Higher stiffness values, e.g., greater than about 85 N/mm, and lower stiffness values, e.g., less than about 25 N/mm, are also contemplated. The stiffness can be measured according to, for example, the standard protocol of ASTM D638-10 (2010).

Another advantageously improved property of the provided graft materials is their stress at yield. The stress at yield of a graft material is a measure of the amount of stress or force that the material can withstand before it starts to deform or permanently change shape. In the context of tendon repair, a graft material preferably has a stress at yield that is greater than the normal stresses placed on the tendon during movement. If the graft material is not strong enough, it may deform under normal loads, leading to a failed repair and potential re-injury. Additionally, the stress at yield of the graft material can affect the healing process of the repaired tendon. If the graft material is too stiff and does not allow for enough movement, it can impede the healing process and prevent the tendon from properly integrating with the graft. On the other hand, if the graft material is too weak and does not provide enough support, it can lead to excessive strain on the healing tendon and delay or prevent proper healing.

The stress at yield of the provided graft material can be, for example, between about 4 MPa and about 20 MPa, e.g., between about 4 MPa and about 13.6 MPa, between about 5.6 MPa and about 15.2 MPa, between about 7.2 MPa and about 16.8 MPa, between about 8.8 MPa and about 18.4 MPa, or between about 10.4 MPa and about 20 MPa. In terms of lower limits, the graft material stress at yield can be, for example, less than about 20 MPa, e.g., less than about 18.4 MPa, less than about 16.8 MPa, less than about 15.2 MPa, less than about 13.6 MPa, less than about 12 MPa, less than about 10.4 MPa, less than about 8.8 MPa, less than about 7.2 MPa, or less than about 5.6 MPa. In terms of lower limits, the graft material stress at yield can be, for example, greater than about 4 MPa, e.g., greater than about 5.6 MPa, greater than about 7.2 MPa, greater than about 8.8 MPa, greater than about 10.4 MPa, greater than about 12 MPa, greater than about 13.6 MPa, greater than about 15.2 MPa, greater than about 16.8 MPa, or greater than about 18.4 MPa. Higher stresses at yield, e.g., greater than about 20 MPa, and lower strains at yield, e.g., less than about 4 MPa, are also contemplated. The stresses at yield can be measured according to, for example, the standard protocol of ASTM D638-10 (2010).

Another advantageously improved property of the provided graft materials is their strain at yield. The strain at yield of a graft material measures the maximum deformation that the graft can undergo before it starts to lose its elasticity and function. A high strain at yield indicates that the graft can tolerate more elongation without permanent damage. A low strain at yield indicates that the graft is more brittle and prone to rupture under loading. A graft material with a high strain at yield can provide better flexibility and adaptability to the tendon repair site, but it may also cause more creep and laxity over time.

The strain at yield of the provided graft material can be, for example, between about 5% and about 20%, e.g., between about 5% and about 14%, between about 6.5% and about 15.5%, between about 8% and about 17%, between about 9.5% and about 18.5%, or between about 11% and about 20%. In terms of upper limits, the graft material strain at yield can be, for example, less than about 20%, e.g., less than about 18.5%, less than about 17%, less than about 15.5%, less than about 14%, less than about 12.5%, less than about 11%, less than about 9.5%, less than about 8%, or less than about 6.5%. In terms of lower limits, the graft material strain at yield can be, for example, greater than about 5%, e.g., greater than about 6.5%, greater than about 8%, greater than about 9.5%, greater than about 11%, greater than about 12.5%, greater than about 14%, greater than about 15.5%, greater than about 17%, or greater than about 18.5%. Higher strains at yield, e.g., greater than about 20%, and lower strains at yield, e.g., less than about 5%, are also contemplated. The strain at yield can be measured according to, for example, the standard protocol of ASTM D638-10 (2010).

Another advantageously improved property of the provided graft materials is their peeling force. The peeling force of a graft material is a measure of resistance of the graft to shear forces that may cause elements of the material to detach from one another. A high peeling force means that the core and shell of the graft can adhere strongly and prevent failure. A low peeling force means that the graft is more vulnerable to rupture.

The 90-degree peel test peeling force of the provided graft material can be, for example, between about 4 N and about 19 N, e.g., between about 4 N and about 7.6 N, between about 4.6 N and about 8.2 N, between about 5.2 N and about 8.8 N, between about 5.8 N and about 9.4 N, or between about 6.4 N and about 10 N. In terms of lower limits, the graft material 90-degree peel test peeling force can be, for example, less than about 10 N, e.g., less than about 9.4 N, less than about 8.8 N, less than about 8.2 N, less than about 7.6 N, less than about 7 N, less than about 6.4 N, less than about 5.8 N, less than about 5.2 N, or less than about 4.6 N. In terms of lower limits, the graft material 90-degree peel test peeling force can be, for example, greater than about 4 N, e.g., greater than about 4.6 N, greater than about 5.2 N, greater than about 5.8 N, greater than about 6.4 N, greater than about 7 N, greater than about 7.6 N, greater than about 8.2 N, greater than about 8.8 N, or greater than about 9.4 N. Higher 90-degree peel test peeling force values, e.g., greater than about 10 N, and lower 90-degree peel test peeling force values, e.g., less than about 4 N, are also contemplated.

The 90-degree peel test peak load of the provided graft material can be, for example, between about 10 N and about 40 N, e.g., between about 10 N and about 28 N, between about 13 N and about 31 N, between about 16 N and about 34 N, between about 19 N and about 37 N, or between about 22 N and about 40 N. In terms of lower limits, the graft material 90-degree peel test peak load can be, for example, less than about 40 N, e.g., less than about 37 N, less than about 34 N, less than about 31 N, less than about 28 N, less than about 25 N, less than about 22 N, less than about 19 N, less than about 16 N, or less than about 13 N. In terms of lower limits, the graft material 90-degree peel test peak load can be, for example, greater than about 10 N, e.g., greater than about 13 N, greater than about 16 N, greater than about 19 N, greater than about 22 N, greater than about 25 N, greater than about 28 N, greater than about 31 N, greater than about 34 N, or greater than about 37 N. Higher 90-degree peel test peak loads, e.g., greater than about 40 N, and lower 90-degree peel test peak loads, e.g., less than about 10 N, are also contemplated.

The shell of the provided graft material includes a photocrosslinkable hydrogel. The photocrosslinkinable hydrogel can be selected to provided the graft material with desirable biocompatibility, biodegradability, non-immunogenicity, mechanical strength, and/or water content. In some embodiments, the photocrosslinkable hydrogel is a polyethylene glycol (PEG) hydrogel. In some embodiments, the photocrosslinkable hydrogel is a hyaluronic acid hydrogel.

In some embodiments, the photocrosslinkable hydrogel is a poly(ethylne glycol)-diaciylate (PEGDA) hydrogel. In some embodiments, the photocrosslinkable hydrogel is a chitosan hydrogel. In some embodiments, the photocrosslinkable hydrogel is a gelatin hydrogel.

In some embodiments, the photocrossslinkable hydrogel includes gelatin or a derivative thereof. In some embodiments, the photocrosslinkable hydrogel includes a gelatin acryloyl. In some embodiments, the photocrosslinkable hydrogel includes gelatin methacrylamide (GelMAm). In some embodiments, the photocrosslinkable hydrogel includes gelatin methacryloyl (GelMA).

The shell of the provided graft material further includes an extracellular matrix. The extracellular matrix can be selected to provide the graft material with improved biocompatibility. The extracellular matrix of the graft material can enhance compatibility of the graft with the surrounding tissue, which can improve the healing process and reduce the risk of rejection or adverse reactions. The extracellular matrix can be selected to increase the ability of the graft material to promote cell attachment, proliferation, and/or differentiation, accelerating proper integration of the graft with the surrounding tissue. The extracellular matrix can be selected to enhance the body's natural healing processes, promoting the regeneration of damaged tissue and the repair of the tendon.

In some embodiments, the extracellular matrix of the provided graft material is a tissue-derived extracellular matrix. Tissue extracellular matrix can advantageously promote corresponding tissue specific differentiation of stem cells, (e.g., mesenchymal stem cells). In some embodiments, the extracellular matrix includes or consists of small intestinal submucosa (SIS). In some embodiments, the extracellular matrix includes or consists of decellularized dermis. In some embodiments the extracellular matrix includes or consists of decellularized liver. In some embodiments, the extracellular matrix includes or consists of decellularized heart valve. In some embodiments, the extracellular matrix includes or consists of decellularized tendon.

In some embodiments, the extracellular matrix of the provided graft material is a cell-derived extracellular matrix. In some embodiments, the extracellular matrix includes or consists of decellularized cell sheets. In some embodiments, the extracellular matrix includes or consists of stem cell-derived extracellular matrix, e.g., mesenchymal stem cell (MSC)-derived extracellular matrix. In some embodiments, the extracellular matrix includes or consists of decellularized extracellular vesicles. In some embodiments, the extracellular matrix includes or consists of tumor cell-derived extracellular matrix.

In some embodiments, the extracellular matrix of the provided graft material is a urea-extracted matrix. Extracellular matrix extracted by this approach can advantageously retain a large number of non-collagenous matrix proteins, in contrast to extracellular matrix extracted using traditional acid-pepsin digestion resulting in mostly collagens. The non-collagenous content of urea-extracted extracellular matrix can beneficially maintain precise, tissue-specific regenerative bioactivity in the graft material. More particularly, urea extraction can preserve pepsin-sensitive, non-collagenous components that can act in combination to contribute to the pro-tenogenesis bioactivity of tendon extracellular matrix. In some embodiments, the extracellular matrix is a non-collagenous matrix enriched extracellular matrix fraction. For example, the extracellular matrix can be a non-collagenous matrix enriched fraction obtained using a gelation-based high speed centrifugation protocol.

The extracellular matrix can include a number of non-collagenous components shown to be advantageous in promoting tenogenesis. In some embodiments, the extracellular matrix includes at least one of fibronectin, decorin, biglycan, fibromodulin, keratocan, prolargin, cartilage oligomeric matrix protein, thrombospondin 1, thrombospondin 4, TGF-β1, TGF-β3, IGF-1, and FGF-2. In some embodiments, the extracellular matrix includes each of fibronectin, decorin, biglycan, fibromodulin, keratocan, prolargin, cartilage oligomeric matrix protein, thrombospondin 1, thrombospondin 4, TGF-β1, TGF-β, IGF-1, and FGF-2. In some embodiments, the extracellular matrix is supplemented with one or more exogenous growth factors. In some embodiments, the extracellular matrix is supplemented with one or more exogenous tenogenic growth factors.

In some embodiments, the extracellular matrix of the provided graft material is present in the form of a coating on the core portion of the graft material. In some embodiments, the extracellular matrix is an extracellular matrix powder added to, and reconstituted in, the photocrosslinkable hydrogel, such that the photocrosslinkable hydrogel includes the extracellular matrix. The amount of extracellular material in the photocrosslinkable hydrogel can be selected to provide the graft material with its advantageous properties, e.g., its ability to offer biological cues supporting tissue regeneration and/or repair, and its desirable load-bearing properties. The concentration of the extracellular matrix in the photocrosslinkable hydrogel can be, for example, between about 0.2 mg/mL and about 10 mg/mL, e.g., between about 0.2 mg/mL and about 2.1 mg/mL, between about 0.3 mg/mL and about 3.1 mg/mL, between about 0.44 mg/mL and about 4.6 mg/mL, between about 0.65 mg/mL and about 6.8 mg/mL, or between about 0.96 mg/mL and about 10 mg/mL. In terms of upper limits, the extracellular matrix concentration in the photocrosslinkable hydrogel can be, for example, less than about 10 mg/mL, e.g., less than about 6.8 mg/mL, less than about 4.6 mg/mL, less than about 3.1 mg/mL, less than about 2.1 mg/mL gel, less than about 1.4 mg/mL, less than about 0.96 mg/mL, less than about 0.65 mg/mL, less than about 0.44 mg/mL, or less than about 0.3 mg/mL. In terms of lower limits, the extracellular matrix concentration in the photocrosslinkable hydrogel can be, for example, greater than about 0.2 mg/mL, e.g., greater than about 0.3 mg/mL, greater than about 0.44 mg/mL, greater than about 0.65 mg/mL, greater than about 0.96 mg/mL, greater than about 1.4 mg/mL, greater than about 3.1 mg/mL, greater than about 4.6 mg/mL, or greater than about 6.8 mg/mL. Higher extracellular matrix concentrations, e.g., greater than about 10 mg/mL, and lower extracellular matrix concentrations, e.g., less than about 0.2 mg/mL, are also contemplated.

The elastomeric polymer of the core portion of the provided graft material can be selected to result in a graft having desired mechanical properties. For example, the elastomeric polymer can be selected to allow the graft material to approximate approximate human tendon-like biomechanical attributes, making the graft particularly advantageous for the clinical repair of large tendon defects. In some embodiments, the elastomeric polymer includes or consists of a silicone elastomer. In some embodiments, the elastomeric polymer includes or consists of a natural rubber latex. In some embodiments, the elastomeric polymer includes or consists of polydimethylsiloxane (PDMS). In some embodiments, the elastomeric polymer includes or consists of polyisoprene. In some embodiments, the elastomeric polymer includes or consists of a polyurethane elastomer.

In some embodiments, the elastomeric polymer of the provided graft material includes a reaction product of a polyol, a polyisocyanate, and an acrylate. The identity and amount of the polyol in the elastomeric polymer of the provided graft material can be selected to provide the graft material with a desired advantageous crosslinking density, hardness and modulus, tensile strength and elongation, and/or biocompatibility. In some embodiments, the polyol of the elastomeric polymer includes or consists of N,N,N′,N′-Tetrakis(2-hydroxypropyl)ethylenediamine, also known as (ethylenedinitrilo)tetra-2-propanol and QUADROL®. In some embodiments, the polyol of the elastomeric polymer includes or consists of triethanolamine. In some embodiments, the polyol of the elastomeric polymer includes or consists of triisopropanolamine. In some embodiments, the polyol of the elastomeric polymer includes or consists of 1-[N,N-Bis(2-hydroxyethyl)amino]-2-propanol. In some embodiments, the polyol of the elastomeric polymer includes or consists of 4-[N,N-Bis(2-hydroxyethyl)amino]benzaldehyde. In some embodiments, the polyol is an oven-dried polyol.

The molar fraction of the polyol in the elastomeric polymer can be, for example, between about 15% and about 60%, e.g., between about 15% and about 42%, between about 19.5% and about 46.5%, between about 24% and about 51%, between about 28.5% and about 55.5%, or between about 33% and about 60%. In terms of lower limits, the polyol molar fraction in the elastomeric polymer can be, for example, less than about 60%, e.g., less than about 55.5%, less than about 51%, less than about 46.5%, less than about 42%, less than about 37.5%, less than about 33%, less than about 28.5%, less than about 24%, or less than about 19.5%. In terms of lower limits, the polyol molar fraction in the elastomeric polymer can be, for example, greater than about 15%, e.g., greater than about 19.5%, greater than about 24%, greater than about 28.5%, greater than about 33%, greater than about 37.5%, greater than about 42%, greater than about 46.5%, greater than about 51%, or greater than about 55.5%. Higher polyol molar fractions, e.g., greater than about 60%, and lower polyol molar fractions, e.g, less than about 15%, are also contemplated.

The identity and amount of the polyisocyanate in the elastomeric polymer of the provided graft material can be selected to provide the graft material with a desired advantageous crosslinking density, chemical resistance, temperature stability, and/or adhesion. In some embodiments, the polyisocyanate of the elastomeric polymer includes or consists of hexamethylene diisocyanate. In some embodiments, the polyisocyanate of the elastomeric polymer includes or consists of isophorone diisocyanate. In some embodiments, the polyisocyanate of the elastomeric polymer includes or consists of methylene dicyclohexyl diisocyanate. In some embodiments, the polyisocyanate of the elastomeric polymer includes or consists of 2,4-diisocyanatotoluene. In some embodiments, the polyisocyanate of the elastomeric polymer includes or consists of 4,4′-methylene bis-(cyclohexylisocyanate). In some embodiments, the polyisocyanate of the elastomeric polymer includes or consists of hexamethylene diisocyanate biuret. In some embodiments, the polyisocyanate of the elastomeric polymer includes or consists of hexamethylene diisocyanate isocyanurate trimer. In some embodiments, the polyisocyanate of the elastomeric polymer includes or consists of hexamethylene diisocyanate isocyanurate trimer. In some embodiments, the polyisocyanate of the elastomeric polymer includes or consists of hexamethylene diisocyanate uretdione. In some embodiments, the polyisocyanate of the elastomeric polymer includes or consists of poly(hexamethylene diisocyanate). In some embodiments, the polyisocyanate of the elastomeric polymer includes or consists of isophorone diisocyanate trimer. In some embodiments, the polyisocyanate of the elastomeric polymer includes or consists of 1,3-cyclohexane. In some embodiments, the polyisocyanate of the elastomeric polymer includes or consists of bis(methylisocyanate). In some embodiments, the polyisocyanate of the elastomeric polymer includes or consists of 2,2,4-trimethyl-hexamethylene diisocyanate.

The molar fraction of the polyisocyanate in the elastomeric polymer can be, for example, between about 25% and about 75%, e.g., between about 25% and about 55%, between about 30% and about 60%, between about 35% and about 65%, between about 40% and about 70%, or between about 45% and about 75%. In terms of lower limits, the polyisocyanate molar fraction in the elastomeric polymer can be, for example, less than about 75%, e.g., less than about 70%, less than about 65%, less than about 60%, less than about 55%, less than about 50%, less than about 45%, less than about 40%, less than about 35%, or less than about 30%. In terms of lower limits, the polyisocyanate molar fraction in the elastomeric polymer can be, for example, greater than about 25%, e.g., greater than about 30%, greater than about 35%, greater than about 40%, greater than about 45%, greater than about 50%, greater than about 55%, greater than about 60%, greater than about 65%, or greater than about 70%. Higher polyisocyanate molar fractions, e.g., greater than about 75%, and lower polyisocyanate molar fractions, e.g, less than about 25%, are also contemplated.

The identity and amount of the acrylate in the elastomeric polymer of the provided graft material can be selected to provide the graft material with a desired advantageous flexibility, abrasion resistance, and/or adhesion. In some embodiments, the acrylate of the elastomeric polymer includes or consists of methacrylic anhydride. In some embodiments, the acrylate of the elastomeric polymer includes or consists of methyl acrylate. In some embodiments, the acrylate of the elastomeric polymer includes or consists of ethyl acrylate. In some embodiments, the acrylate of the elastomeric polymer includes or consists of methyl methacrylate. In some embodiments, the acrylate of the elastomeric polymer includes or consists of acrylic anhydride. In some embodiments, the acrylate of the elastomeric polymer includes or consists of acrylamide. In some embodiments, the acrylate of the elastomeric polymer includes or consists of methacrylamide. In some embodiments, the acrylate of the elastomeric polymer includes or consists of methacrylic acid.

The molar fraction of the acrylate in the elastomeric polymer can be, for example, between about 5% and about 40%, e.g., between about 5% and about 26%, between about 8.5% and about 29.5%, between about 12% and about 33%, between about 15.5% and about 36.5%, or between about 19% and about 40%. In terms of lower limits, the acrylate molar fraction in the elastomeric polymer can be, for example, less than about 40%, e.g., less than about 36.5%, less than about 33%, less than about 29.5%, less than about 26%, less than about 22.5%, less than about 19%, less than about 15.5%, less than about 12%, or less than about 8.5%. In terms of lower limits, the acrylate molar fraction in the elastomeric polymer can be, for example, greater than about 5%, e.g., greater than about 8.5%, greater than about 12%, greater than about 15.5%, greater than about 19%, greater than about 22.5%, greater than about 26%, greater than about 29.5%, greater than about 33%, or greater than about 36.5%. Higher acrylate molar fractions, e.g., greater than about 40%, and lower acrylate molar fractions, e.g, less than about 5%, are also contemplated.

In some embodiments, the elastomeric polymer is crosslinked to the shell of the provided graft material by a photocrosslinking agent. The identity of the photocrosslinking agent can be selected to provide the graft material with desired crosslinking properties. In some embodiments, the photocrosslinking agent includes or consists of benzophenone. In some embodiments, the photocrosslinking agent includes or consists of one or more iodonium salts. In some embodiments, the photocrosslinking agent includes or consists of one or more acetophenone derivatives. In some embodiments, the photocrosslinking agent includes or consists of one or more anthraquinone derivatives. In some embodiments, the photocrosslinking agent includes or consists of one or more triazines.

In some embodiments, the provided graft material further includes cells seeded within the shell of the graft material. In some embodiments, the graft material includes cells seeded on the shell. The cells seeded in and/or on the shell can include, for example, and without limitation, adipose-derived stem cells, tenocytes, mesenchymal stem cells, fibroblasts, chondrocytes, or any combination thereof.

### IV. Methods for Repairing Tendon Defects

Another aspect of the present disclosure relates to methods for repairing a tendon defect in a subject. The particular features of the methods have been shown to provide a subject with treatments benefiting from the enhanced characteristics of the graft materials disclosed herein. The methods generally include implanting a graft material in a subject. The graft material can be any of those discussed above. The graft material can be implanted in the subject at a location proximate to a tendon defect of the subject, e.g., at the site of the defect of the tendon.

In some embodiments, the method further includes introducing or adhering one or more types of cells to the graft material. The introduction or adhering of the cells to the scaffold can include seeding cells onto the scaffold prior to or subsequent to implantation in a subject. In some embodiments, one or more cell types promoting tendon repair are introduced or adhered to the graft material. The cell types can be any of those disclosed herein. For example, the cells introduced or adhered to the graft material can include adipose-derived stem cells, mesenchymal stem cells, or any combination thereof.

The tendon defect of the subject can be the result of, for example, a trauma, an injury, overuse, a degenerative disease, aging, or a genetic disorder. Accordingly, the provided graft material can be implemented in treatments related to, for example, sports injuries or elder care. In some embodiments, the tendon defect site includes a rotator cuff tear. Tendon defects that can be repaired with the provided graft material include defects in the supraspinatus tendon. Exemplary tendon defects include diseased, degenerated, or damaged, e.g., torn, tendons.

In some embodiments, the method further includes evaluating the subject to determine the nature of the tendon defect that requires repair, and the characteristics of the graft material appropriate to treat the subject. The evaluating of the subject can include medical imaging, such as X-ray imaging, MRI scans, or CT scans, which can provide dimensions of the tendon defect site, and can be utilized for determining the desired configuration, such as size and/or shape, of the graft material.

### V. Methods for Preparing Films

Another aspect of the present disclosure relates to methods for preparing the provided graft materials. The particular combinations of steps of the methods have been show to impart the graft materials with the advantageous features discussed above, including high mechanical strength and integrity, shape retention, and beneficial biological activity. The methods generally include providing an extracellular matrix. The extracellular matrix can be any of those disclosed herein. For example, in some embodiments, the extracellular matrix includes a cell-derived extracellular matrix or a tissue-derived extracellular matrix, e.g., a tendon extracellular matrix. In some embodiments, the extracellular matrix is a urea-extracted extracellular matrix. In some embodiments, the providing of the extracellular matrix also includes supplementing the extracellular matrix with one or more exogenous growth factors, e.g., one or more exogenous tenogenic growth factors.

In some embodiments, the providing of the extracellular matrix includes providing a non-collagenous matrix enriched fraction of an extracellular matrix. For example, in some embodiments, the method includes incubating an extracellular matrix at a temperature that promotes gelation of a collagen matrix enriched portion of the extracellular matrix, and then removing this collagen matrix-enriched portion, thereby leaving a non-collagenous matrix enriched fraction suitable for use in preparing the provided graft material. For example, gelation-based fractionation methods can utilize the thermally-dependent crosslinking property of collagen to separate the collagenous matrix and non-collagenous matrix from one another in tendon extracellular matrix. In some embodiments, the removing of the collagen-enriched portion includes centrifuging the extracellular matrix subsequent to the gelation.

In some embodiments, the provided method further includes selecting the temperature of the gelation conditions. The temperature of the gelation can influence which components of the extracellular matrix are included in the gel portion, and can determine which components, if any, are denatured. The gelation temperature can be, for example, between about 30° C. and about 45° C., e.g., between about 30° C. and about 39° C., between about 31.5° C. and about 40.5° C., between about 33° C. and about 42° C., between about 34.5° C. and about 43.5° C., or between about 36° C. and about 45° C. In terms of upper limits, the gelation temperature can be, for example, less than about 45° C., e.g., less than about 43.5° C., less than about 42° C., less than about 40.5° C., less than about 39° C., less than about 37.5° C., less than about 36° C., less than about 34.5° C., less than about 33° C., or less than about 31.5° C. In terms of lower limits, the gelation temperature can be, for example, greater than about 30° C., e.g., greater than about 31.5° C., greater than about 33° C., greater than about 34.5° C., greater than about 36° C., greater than about 37.5° C., greater than about 39° C., greater than about 40.5° C., greater than about 42° C., or greater than about 43.5° C. Higher gelation temperatures, e.g., greater than about 45° C., and lower gelation temperatures, e.g., less than about 30° C., are also contemplated. In some embodiments, the provided method further includes selecting the duration of the gelation conditions.

In some embodiments, the provided method for producing a graft material also includes forming a pre-hydrogel mixture with the extracellular matrix and a photocrosslinkable polymer. The photocrosslinkable polymer of the method can be any of those disclosed herein. For example, in some embodiments, the photocrosslinkable polymer includes or consists of gelatin, or a derivative thereof, e.g., a gelatin acryloyl such as gelatin methacryloyl.

In some embodiments, the provided method for producing a graft material also includes providing an elastomeric polymer. The elastomeric polymer of the method can be any of those disclosed herein. For example, in some embodiments, the elastomeric polymer includes a polyurethane elastomer. In some embodiments, the providing of the elastomer includes forming a pre-elastomer mixture with a polyol, a polyisocyanate, and an acrylate, and then irradiating the pre-elastomer mixture with ultraviolet light. The polyol, polyisocyanate, and acrylate of the method can be any of those disclosed herein. For example, in some embodiments, the polyol includes or consists of N,N,N′,N′-Tetrakis(2-hydroxypropyl)ethylenediamine, the polyisocyanate includes or consists of hexamethylene diisocyanate, and the acrylate includes or consists of methacrylic anhydride. In some embodiments, method further includes drying the polyol, e.g., the N,N,N′,N′-Tetrakis(2-hydroxypropyl)ethylenediamine, in an oven before forming the pre-elastomer mixture. Such drying of the polyol can in some instances improve the mechanical strength of the resulting elastomeric polymer and graft material.

In some embodiments, the provided method for producing a graft material also includes absorbing a photcrosslinking agent onto a surface of the elastomeric polymer, creating a treated elastomeric polymer. The photocrosslinking agent of the method can be any of those disclosed herein. For example, in some embodiments, the photocrosslinking agent includes or consists of benzophenone.

In some embodiments, the provided method for producing a graft material also includes applying the prehydrogel mixture to the treated elastomeric material, creating a pre-graft material. In some embodiments, the method also includes irradiating the pre-graft material with ultraviolet light for an exposure duration, producing the graft material. In some embodiments, the exposure duration is selected to be long enough to create robust bonding between the hydrogel of the graft material shell, and the elastomeric polymer of the hydrogel core, while also being short enough to minimize interference with the bioactivity of the ECM-hydrogel and with the mechanical features of the elastomer. For example, previously reported strategies for crosslinking a hydrogel and elastomer yielded extremely tough and functional bonding, but also required a long time (e.g., one hour) of exposure to UV irradiation, which may damage elastomer strength, extracellular matrix bioactivity, or hydrogel features.

In some embodiments, the particular choice of the hydrogel shell and the elastomeric polymer core allow a benzophenone treatment to enhance the crosslinking efficacy of anhydride carbonyl groups shared by both the hydrogel and the elastomeric polymer. This enhanced crosslinking efficacy permit the UV exposure duration to be significantly reduced. The exposure duration can be, for example, between about 0.2 min and about 10 min, e.g., between about 0.2 min and about 2.1 min, between about 0.3 min and about 3.1 min, between about 0.44 min and about 4.6 min, between about 0.65 min and about 6.8 min, or between about 0.96 min and about 10 min. In terms of upper limits, the exposure duration can be, for example, less than about 10 min, e.g., less than about 6.8 min, less than about 4.6 min, less than about 3.1 min, less than about 2.1 min, less than about 1.3 min, less than about 0.96 min, less than about 0.65 min, less than about 0.44 min, or less than about 0.3 min. In terms of lower limits, the exposure duration can be, for example, greater than about 0.2 min, e.g., greater than about 0.3 min, greater than about 0.44 min, greater than about 0.65 min, greater than about 0.96 min, greater than about 1.4 min, greater than about 2.1 min, greater than about 3.1 min, greater than about 4.6 min, or greater than about 6.8 min. Longer exposure durations, e.g., greater than about 10 min, and shorter exposure durations, e.g., less than about 0.2 min, are also contemplated.

In some embodiments, the provided method for producing a graft material also includes seeding cells within or onto the graft material. The cells can be any of those disclosed herein. For example, in some embodiments, the cells include or consist of stem cells, e.g., adipose-derived stem cells, mesenchymal stem cells, or a combination thereof.

### VI. Exemplary Embodiments

The following embodiments are contemplated. All combinations of features and embodiments are contemplated.

Embodiment 1: A graft material comprising: a shell comprising a photocrosslinkable hydrogel and an extracellular matrix; and a core comprising an elastomeric polymer covalently crosslinked to the shell.

Embodiment 2: An embodiment of embodiment 1, wherein the extracellular matrix comprises a tissue-derived extracellular matrix.

Embodiment 3: An embodiment of embodiment 1 or 2, wherein the extracellular matrix comprises a cell-derived extracellular matrix.

Embodiment 4: An embodiment of any embodiment of embodiments 1-3, wherein the extracellular matrix is supplemented with a growth factor.

Embodiment 5: An embodiment of embodiment 4, wherein the growth factor is a tenogenic growth factor.

Embodiment 6: An embodiment of any embodiment of embodiments 1-5, wherein the photocrosslinkable hydrogel comprises the extracellular matrix.

Embodiment 7: An embodiment of embodiment 6, wherein the concentration of the extracellular matrix in the photocrosslinkable hydrogel is between about 0.2 mg/mL and about 10 mg/mL.

Embodiment 8: An embodiment of any embodiment of embodiments 1-6, wherein the extracellular matrix is coated on a surface of the core.

Embodiment 9: An embodiment of any embodiment of embodiments 1-8, wherein the extracellular matrix comprises a urea-extracted extracellular matrix, or a non-collagenous matrix enriched fraction thereof.

Embodiment 10: An embodiment of any embodiment of embodiments 1-9, wherein the extracellular matrix comprises a tendon extracellular matrix.

Embodiment 11: An embodiment of any embodiment of embodiments 1-10, wherein the elastomeric polymer comprises a polyurethane elastomer.

Embodiment 12: An embodiment of any embodiment of embodiments 1-11, wherein the elastomeric polymer comprises a reaction product of a polyol, a polyisocyanate, and an acrylate.

Embodiment 13: An embodiment of embodiment 12, wherein the polyol comprises N,N,N′,N′-Tetrakis(2-hydroxypropyl)ethylenediamine, triethanolamine, triisopropanolamine, 1-[N,N-Bis(2-hydroxyethyl)amino]-2-propanol, 4-[N,N-Bis(2-hydroxyethyl)amino]benzaldehyde, or a combination thereof.

Embodiment 14: An embodiment of embodiment 12 or 13, wherein the polyol is an oven-dried polyol.

Embodiment 15: An embodiment of any embodiment of embodiments 12-14, wherein the polyisocyanate comprises hexamethylene diisocyanate, isophorone diisocyanate, methylene dicyclohexyl diisocyanate, 2,4-diisocyanatotoluene, 4,4′-methylene bis-(cyclohexylisocyanate), hexamethylene diisocyanate biuret, hexamethylene diisocyanate isocyanurate trimer, hexamethylene diisocyanate uretdione, poly(hexamethylene diisocyanate), isophorone diisocyanate trimer, 1,3-cyclohexane, bis(methylisocyanate), 2,2,4-trimethyl-hexamethylene diisocyanate, or a combination thereof.

Embodiment 16: An embodiment of any embodiment of embodiments 12-15, wherein the acrylate comprises methacrylic anhydride, methyl acrylate, ethyl acrylate, methyl methacrylate, acrylic anhydride, acrylamide, methacrylamide, acrylic acid, methacrylic acid, or a combination thereof.

Embodiment 17: An embodiment of any embodiment of embodiments 12-16, wherein the molar fraction of the polyol in the elastomeric polymer is between about 15% and about 60%.

Embodiment 18: An embodiment of any embodiment of embodiments 12-17, wherein the molar fraction of the polyisocyanate in the elastomeric polymer is between about 25% and about 75%.

Embodiment 19: An embodiment of any embodiment of embodiments 12-18, wherein the molar fraction of the acrylate in the elastomeric polymer is between about 5% and about 40%.

Embodiment 20: An embodiment of any embodiment of embodiments 1-19, wherein the elastomeric polymer comprises a reaction product of N,N,N′,N′-Tetrakis(2-hydroxypropyl)ethylenediamine, hexamethylene diisocyanate, and methacrylic anhydride.

Embodiment 21: An embodiment of any embodiment of embodiments 1-20, wherein the extracellular matrix comprises fibronectin, decorin, biglycan, fibromodulin, keratocan, prolargin, cartilage oligomeric matrix protein, thrombospondin 1, thrombospondin 4, TGF-$1, TGF-P3, IGF-1, FGF-2, or a combination thereof.

Embodiment 22: An embodiment of any embodiment of embodiments 1-21, wherein the extracellular matrix comprises fibronectin, decorin, biglycan, fibromodulin, keratocan, prolargin, cartilage oligomeric matrix protein, thrombospondin 1, thrombospondin 4, TGF-P1, TGF-β3, IGF-1, and FGF-2.

Embodiment 23: An embodiment of any embodiment of embodiments 1-22, wherein the photocrosslinkable hydrogel comprises gelatin or a derivative thereof.

Embodiment 24: An embodiment of embodiment 23, wherein the photocrosslinkable hydrogel comprises a gelatin acryloyl.

Embodiment 25: An embodiment of embodiment 24, wherein the photocrosslinkable hydrogel comprises gelatin methacryloyl.

Embodiment 26: An embodiment of any embodiment of embodiments 1-25, wherein the elastomeric polymer is crosslinked to the shell by a photocrosslinking agent.

Embodiment 27: An embodiment of embodiment 26, wherein the photocrosslinking agent comprises benzophenone.

Embodiment 28: An embodiment of any embodiment of embodiments 1-27, wherein the graft material further comprises: cells seeded in or on the shell.

Embodiment 29: An embodiment of embodiment 28, wherein the cells comprise stem cells.

Embodiment 30: An embodiment of embodiment 29, wherein the cells comprise adipose-derived stem cells, mesenchymal stem cells, or a combination thereof.

Embodiment 31: An embodiment of any embodiment of embodiments 1-30, wherein the graft material exhibits a tensile modulus between about 100 MPa and about 1000 MPa, as measured by ASTM D638-10 (2010).

Embodiment 32: An embodiment of any embodiment of embodiments 1-31, wherein the graft material exhibits a stiffness between about 25 N/mm and about 85 N/mm, as measured by ASTM D638-10 (2010).

Embodiment 33: An embodiment of any embodiment of embodiments 1-32, wherein the graft material exhibits a stress at yield between about 4 MPa and about 20 MPa, as measured by ASTM D638-10 (2010).

Embodiment 34: An embodiment of any embodiment of embodiments 1-33, wherein the graft material exhibits a strain at yield between about 5% and about 20%, as measured by ASTM D638-10 (2010).

Embodiment 35: An embodiment of any embodiment of embodiments 1-34, wherein the graft material exhibits a 90-degree peel test peeling force between about 4 N and about 10 N, as measured by ASTM D638-10 (2010).

Embodiment 36: An embodiment of any embodiment of embodiments 1-35, wherein the graft material exhibits a 90-degree peel test peak load between about 10 N and about 40 N, as measured by ASTM D638-10 (2010).

Embodiment 37: A method of repairing a defect of a tendon of a subject, the method comprising implanting the graft material of any embodiment of embodiments 1-36 in the subject proximate to the defect of the tendon.

Embodiment 38: An embodiment of embodiment 37, wherein the defect comprises a rotator cuff tear.

Embodiment 39: An embodiment of embodiment 37 or 38, wherein the tendon comprises a supraspinatus tendon.

Embodiment 40: A method of producing a graft material, the method comprising: providing an extracellular matrix; forming a pre-hydrogel mixture comprising a photocrosslinkable polymer and the extracellular matrix; providing an elastomeric polymer; absorbing a photocrosslinking agent onto a surface of the elastomeric polymer, thereby yielding a treated elastomeric polymer; applying the pre-hydrogel mixture to the treated elastomeric polymer, thereby creating a pre-graft material; and irradiating the pre-graft material with ultraviolet light for an exposure duration, thereby producing the graft material.

Embodiment 41: An embodiment of embodiment 40, wherein the photocrosslinking agent comprises benzophenone.

Embodiment 42: An embodiment of embodiment 40 or 41, wherein the exposure duration is between 0.2 min and 10 min.

Embodiment 43: An embodiment of any embodiment of embodiments 40-42, wherein the providing of the extracellular matrix comprises: incubating the extracellular matrix at a gelation temperature sufficient to promote gelation of a collagen matrix-enriched portion of the extracellular matrix; and removing the collagen matrix-enriched portion from the extracellular matrix.

Embodiment 44: An embodiment of embodiment 43, wherein the gelation temperature is between 30° C. and 45° C.

Embodiment 45: An embodiment of embodiment 43 or 44, wherein the removing of the collagen-enriched portion comprises centrifuging the extracellular matrix.

Embodiment 46: An embodiment of any embodiment of embodiments 40-45, wherein the extracellular matrix comprises a tissue-derived extracellular matrix.

Embodiment 47: An embodiment of any embodiment of embodiments 40-46, wherein the extracellular matrix comprises a cell-derived extracellular matrix.

Embodiment 48: An embodiment of any embodiment of embodiments 40-47, wherein the providing of the extracellular matrix comprises supplementing the extracellular matrix with a growth factor.

Embodiment 49: An embodiment of embodiment 48, wherein the growth factor is a tenogenic growth factor.

Embodiment 50: An embodiment of any embodiment of embodiments 40-42, wherein the extracellular matrix comprises a urea-extracted extracellular matrix.

Embodiment 51: An embodiment of any embodiment of embodiments 40-50, wherein the extracellular matrix comprises a tendon extracellular matrix.

Embodiment 52: An embodiment of any embodiment of embodiments 40-51, wherein the elastomeric polymer comprises a polyurethane elastomer.

Embodiment 53: An embodiment of any embodiment of embodiments 40-52, wherein the providing of the elastomeric polymer comprises: drying a polyol in an oven, thereby generating an oven-dried polyol; forming a pre-elastomer mixture comprising the oven-dried polyol, a polyisocyanate, and an acrylate; and irradiating the pre-elastomer mixture with ultraviolet light, thereby generating the elastomeric polymer.

Embodiment 54: An embodiment of embodiment 53, wherein the polyol comprises N,N,N′,N′-Tetrakis(2-hydroxypropyl)ethylenediamine, triethanolamine, triisopropanolamine, 1-[N,N-Bis(2-hydroxyethyl)amino]-2-propanol, 4-[N,N-Bis(2-hydroxyethyl)amino]benzaldehyde, or a combination thereof.

Embodiment 55: An embodiment of embodiment 53 or 54, wherein the polyisocyanate comprises hexamethylene diisocyanate, isophorone diisocyanate, methylene dicyclohexyl diisocyanate, 2,4-diisocyanatotoluene, 4,4′-methylene bis-(cyclohexylisocyanate), hexamethylene diisocyanate biuret, hexamethylene diisocyanate isocyanurate trimer, hexamethylene diisocyanate uretdione, poly(hexamethylene diisocyanate), isophorone diisocyanate trimer, 1,3-cyclohexane, bis(methylisocyanate), 2,2,4-trimethyl-hexamethylene diisocyanate, or a combination thereof.

Embodiment 56: An embodiment of any embodiment of embodiments 53-55, wherein the acrylate comprises methacrylic anhydride, methyl acrylate, ethyl acrylate, methyl methacrylate, acrylic anhydride, acrylamide, methacrylamide, acrylic acid, methacrylic acid, or a combination thereof.

Embodiment 57: An embodiment of any embodiment of embodiments 53-56, wherein the molar fraction of the polyol in the pre-elastomer mixture is between about 15% and about 60%.

Embodiment 58: An embodiment of any embodiment of embodiments 53-57, wherein the molar fraction of the polyisocyanate in the pre-elastomer mixture is between about 25% and about 75%.

Embodiment 59: An embodiment of any embodiment of embodiments 53-58, wherein the molar fraction of the acrylate in the pre-elastomer mixture is between about 5% and about 40%.

Embodiment 60: An embodiment of any embodiment of embodiments 40-59, wherein the elastomeric polymer comprises a reaction product of N,N,N′,N′-Tetrakis(2-hydroxypropyl)ethylenediamine, hexamethylene diisocyanate, and methacrylic anhydride.

Embodiment 61: An embodiment of any embodiment of embodiments 40-60, wherein the extracellular matrix comprises fibronectin, decorin, biglycan, fibromodulin, keratocan, prolargin, cartilage oligomeric matrix protein, thrombospondin 1, thrombospondin 4, TGF-β1, TGF-β3, IGF-1, FGF-2, or a combination thereof.

Embodiment 62: An embodiment of any embodiment of embodiments 40-61, wherein the extracellular matrix comprises fibronectin, decorin, biglycan, fibromodulin, keratocan, prolargin, cartilage oligomeric matrix protein, thrombospondin 1, thrombospondin 4, TGF-β1, TGF-β3, IGF-1, and FGF-2.

Embodiment 63: An embodiment of any embodiment of embodiments 40-62, wherein the photocrosslinkable polymer comprises gelatin or a derivative thereof.

Embodiment 64: An embodiment of embodiment 63, wherein the photocrosslinkable polymer comprises a gelatin acryloyl.

Embodiment 65: An embodiment of embodiment 64, wherein the photocrosslinkable polymer comprises gelatin methacryloyl.

Embodiment 66: An embodiment of any embodiment of embodiments 40-65, wherein the concentration of the extracellular matrix in the pre-hydrogel mixture is between about 0.2 mg/mL and about 10 mg/mL.

Embodiment 67: An embodiment of any embodiment of embodiments 40-66, wherein the method further comprises: seeding cells in or on the graft material.

Embodiment 68: An embodiment of embodiment 67, wherein the cells comprise stem cells.

Embodiment 69: An embodiment of embodiment 68, wherein the cells comprise adipose-derived stem cells, mesenchymal stem cells, or a combination thereof.

Embodiment 70: An embodiment of any embodiment of embodiments 40-69, wherein the graft material exhibits a tensile modulus between about 100 MPa and about 1000 MPa, as measured by ASTM D638-10 (2010).

Embodiment 71: An embodiment of any embodiment of embodiments 40-70, wherein the graft material exhibits a stiffness between about 25 N/mm and about 85 N/mm, as measured by ASTM D638-10 (2010).

Embodiment 72: An embodiment of any embodiment of embodiments 40-71, wherein the graft material exhibits a stress at yield between about 4 MPa and about 20 MPa, as measured by ASTM D638-10 (2010).

Embodiment 73: An embodiment of any embodiment of embodiments 40-72, wherein the graft material exhibits a strain at yield between about 5% and about 20%, as measured by ASTM D638-10 (2010).

Embodiment 74: An embodiment of any embodiment of embodiments 40-73, wherein the graft material exhibits a 90-degree peel test peeling force between about 4 N and about 10 N, as measured by ASTM D638-10 (2010).

Embodiment 75: An embodiment of any embodiment of embodiments 40-74, wherein the graft material exhibits a 90-degree peel test peak load between about 10 N and about 40 N, as measured by ASTM D638-10 (2010).

Embodiment 76: An extracellular matrix composition comprising a non-collagenous matrix enriched fraction of a urea-extracted extracellular matrix.

Embodiment 77: An embodiment of embodiment 77, wherein the urea-extracted extracellular matrix comprises a urea-extracted tissue-derived extracellular matrix.

Embodiment 78: An embodiment of embodiment 77 or 78, wherein the urea-extracted extracellular matrix comprises a urea-extracted tendon extracellular matrix.

Embodiment 79: An embodiment of embodiment 77, wherein the urea-extracted extracellular matrix comprises a urea-extracted cell-derived extracellular matrix.

Embodiment 80: An embodiment of any embodiment of embodiments 76-79, wherein the extracellular matrix composition is supplemented with a growth factor.

Embodiment 81: An embodiment of embodiment 79, wherein the growth factor comprises a tenogenic growth factor.

Embodiment 82: An embodiment of any embodiment of embodiments 76-81, wherein the extracellular matrix composition has the form of a fluid.

Embodiment 83: An embodiment of any embodiment of embodiments 76-81, wherein the extracellular matrix composition has the form of a lyophilized solid.

Embodiment 84: A hydrogel comprising the extracellular matrix composition of any one of embodiments 77-83.

### EXAMPLES

The present disclosure will be better understood in view of the following non-limiting examples. The following examples are intended for illustrative purposes only and do not limit in any way the scope of the present invention.

### Example 1. Synthesis of Core-Shell Structured Hybrid Scaffold Graft Material

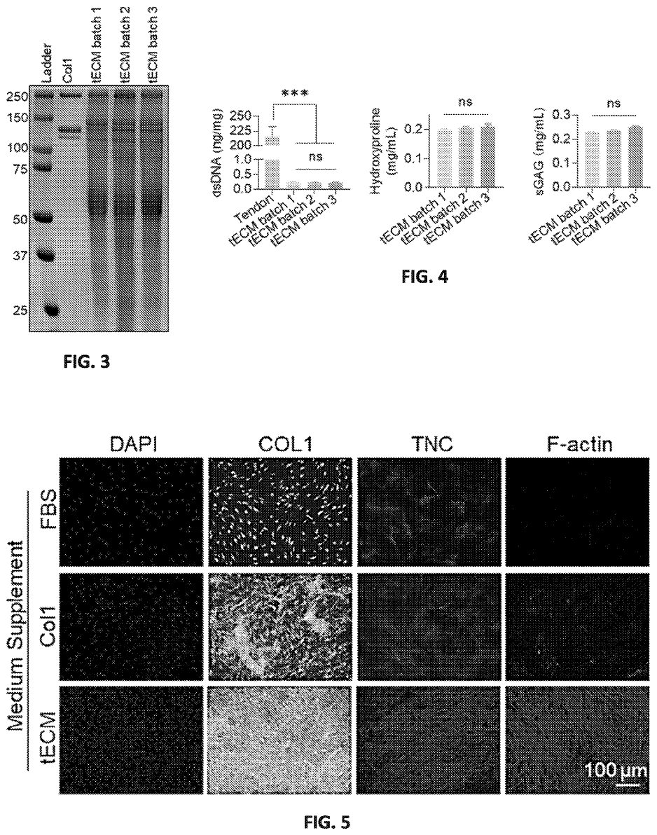

Materials used to synthesize the extracellular matrix hybrid scaffold (ECM-HyS) were systematically characterized as a quality assurance and quality control (QA/QC) step for ECM-HyS manufacturing (FIG. 2). These characterizations included the following. (1) To evaluate the cellularity and protein content of tECM, double stranded DNA (dsDNA) assay, sulfate-polyacrylamide gel electrophoresis (SDS-PAGE), hydroxyproline assay, and sulfated glycosaminoglycan (sGAG) assay were carried out. Data revealed that urea-extracted tendon extracellular matrix (tECM) was DNA-free and contained a number of low molecular weight (<100 kDa) protein components that were absent in commercially available bovine collagen type I solution (Coll) (FIGS. 3 and 4). (2) tECM bioactivity on hASCs was evaluated in 2D culture via assessment of established tenogenic markers using qPCR and immunofluorescence staining.

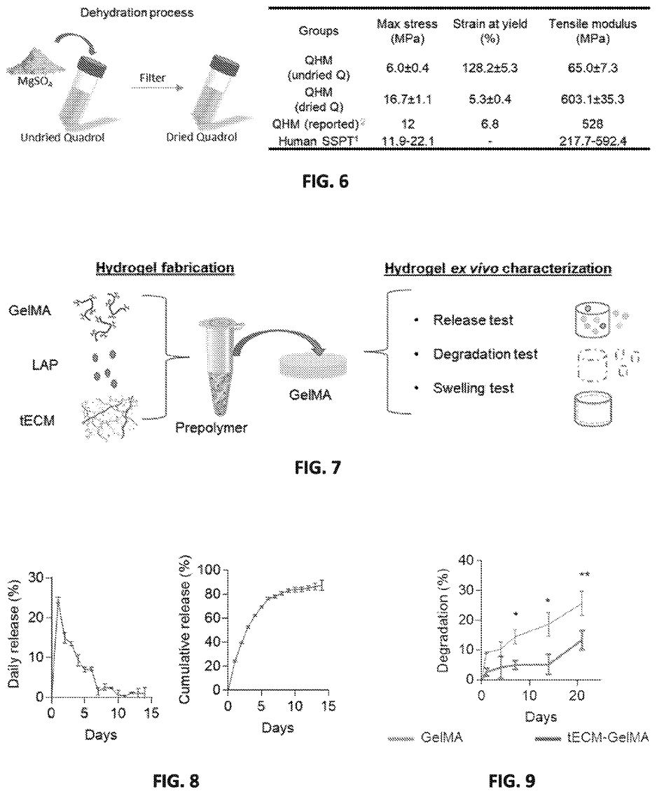

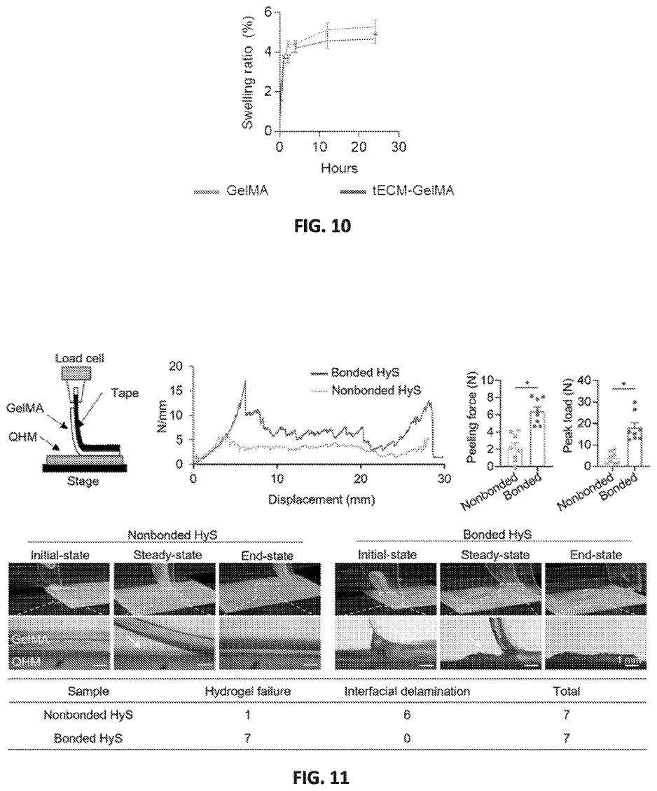

Data showed that tECM enhanced human adipose-derived stem cells (hASC) proliferation and tenogenic differentiation in vitro (FIG. 5). (3) Elastomer QHM biomechanical properties were examined via tensile and suture retention tests. In contrast to QHM elastomer as described in previously published protocols (D. F. E. Ker et al., Adv. Funct. Mater. 28, (2018): 1707107), QHM for the current graft material was fabricated using a modified method that included an extra drying step for quadrol. Without the extra drying step, fabricated QHM exhibited inferior tensile properties (FIG. 6). Without being bound to a particular theory, the inventors believe these inferior tensile properties are likely because atmospheric humidity interfered with the reaction between the hydroxyl group of quadrol and the isocyanate groups of hexamethylene diisocyanate or the anhydride carbonyl group of methacrylic anhydride. With further modification, QHM fabricated using the revised protocol resulted in higher, human tendon-like, mechanical properties (FIG. 6). (4) tECM-GelMA hydrogel was characterized based on its swelling property, degradation, and tECM release tests ex vivo. Data showed that tECM-GeIMA exhibited limited swelling capacity (4.4% swelling ratio) and degradability (25.7% of weight loss after 21 days), and initiated a burst release of tECM within the first 2 days followed by a sustained release profile over a period of 15 days (FIGS. 2-10).

The tECM-HyS construct was formed using a simple yet versatile method to assemble preshaped QHM elastomer and tECM containing GelMA hydrogel into a core-shell structured hybrid construct (tECM-HyS). There are three major components included in tECM-HyS fabrication: tECM, GelMA hydrogel, and QHM elastomer. To obtain soluble tECM extracts, bovine Achilles tendon (AT) from adult bovines was purchased from a commercial market (Shatin, Hong Kong) and subjected to urea-based extraction as described previously (D. Wang et al., FASEB J. 34, (2020): 8172). To prepare GelMA prepolymer solution, 10% GelMA (EFL) with or without tECM (final concentration in hydrogel: 0.6 mg/mL by BCA analysis) was dissolved in PBS with 0.25% (w/v) photoinitiator (lithium acylphospinate salt, LAP; EFL). A UV-crosslinkable polyurethane elastomer called QHM, which is formulated with quadrol (Q), hexamethylene diisocyanate (H), and methacrylic anhydride (M) was developed based on a published protocol (D. F. E. Ker et al. Adv. Funct. Mater. 28, (2018): 1707107) with the modification that an extra drying step for Q was included. To develop tECM-HyS, briefly, the surface of the QHM elastomer was treated by absorbing benzophenone as described previously (H. Yuk, T. Zhang, G. A. Parada, X. Liu & X. Zhao, Nat. Commun. 7, (2016): 12028). Thereafter, the GelMA prepolymer solution was gently applied onto the freshly treated QHM followed by UV irradiation using a UV lamp (EFL; 365 nm, 25 mW/cm²) for 90 s.

SDS-PAGE analysis was performed to evaluate the protein composition of different batches of tECM as described previously (D. Wang et al., FASEB J. 34, (2020): 8172). Protein concentration from each batch of tECM was quantified using BCA kit (Invitrogen). Protein samples with the same concentration (1 mg/mL) were mixed with loading buffer and heated at 95° C. for 5 minutes. The denatured samples were loaded on 8% SDS-PAGE gel, stained with 0.5% of Coomassie blue solution, and imaged using a ChemiDoc Imaging System (Bio-Rad).

A Quant-iT Picogreen dsDNA assay was performed to examine the cellular content in each batch of tECM as described previously (B. B. Rothrauff, G. Yang & R. S. Tuan, Stem Cell Res. Ther. 8, (2017): 133). After extraction, the tECM were digested by papain (Sigma-Aldrich) at 60° C. for 1 hour. The digested tECM samples or dsDNA standard were mixed with Quant-iT Picogreen reagent (Invitrogen) and incubated for 5 minutes at room temperature. Fluorescence was measured at a wavelength of 485/530 nm using microplate reader (SpectraMax i3X) and plotted versus the mass of the DNA component standard.

An sGAG assay was performed in order to quantitatively assess sGAG content of each batch of tECM as described previously (K. Shimomura, B. B. Rothrauff & Tuan, Am. J. Sports Med. 45, (2017): 604). The tECM was digested with 1 mg/mL papain solution. Papain digested samples were reacted using the Glycosaminoglycan Assay Kit (BLYSCAN™) according to the manufacturer's protocol. The absorbance was measured at a wavelength of 656 nm using a microplate reader. Dilutions of provided chondroitin 4-sulfate were used to generate a standard curve.

A hydroxyproline assay was performed to quantify the collagen content in each batch of tECM as described previously (K. Shimomura, B. B. Rothrauff & Tuan, Am. J. Sports Med. 45, (2017): 604). The tECM was digested with 4 N of sodium hydroxide (Sigma-Aldrich). The samples were heated to 120° C. for 20 minutes and then neutralized with 4 N of hydrochloric acid (Sigma-Aldrich). The samples were then oxidized with chloramine-T (Sigma-Aldrich) at room temperature for 20 minutes and reacted with Ehrlich's reagent (Sigma-Aldrich) at 65° C. for 20 minutes. The absorbance was measured at a wavelength of 550 nm using a microplate reader. The hydroxyproline content was calculated based on a calibrated standard curve.

A swelling test was performed to evaluate the swelling behavior of hydrogels. GeIMA and tECM-GelMA hydrogels (round disk: 8 mm (diameter)×1 mm (thickness)) were used for ex vivo swelling test. The swelling behavior of the hydrogel was measured in distilled water at 37° C. At designated time points (20 minutes, 40 minutes, 60 minutes, 2 hours, 4 hours, 8 hours, 24 hours, 48 hours, 72 hours), the hydrogels were removed from the water and weighed after quick blotting with filter paper (Whatman, Sigma-Aldrich). Each swelling value was the average value of three specimens measured at the same time. Swelling rate was calculated using the following formula:

$$Swelling rate ( \% ) = \frac{W s - W d}{W d} \times 100 ,$$

Where Wd refers to the dry weight of hydrogel at day 0 and Ws refers to the swollen weight of hydrogel at designated time points.

GelMA and tECM-GelMA hydrogels (round disk: 8 mm (diameter)×1 mm (thickness)) were used for ex vivo degradation tests. Hydrogel samples were freeze-dried for 24 hours, measured for initial weight (Wi), and then kept in PBS that was refreshed daily in an incubator at 37° C. The specimens were later rinsed with deionized water (dH₂O) and freeze dried at predetermined time intervals and final weight (Wf) were recorded. The degradation rates were calculated using the following formula:

$$Degradation ( \% ) = \frac{W i - W f}{W i} \times 100 \%$$

To investigate the release profile of the tECM from GelMA hydrogels, a modified release study was performed as described previously (U. G. Longo, A. Lamberti, N. Maffulli & V. Denaro, Br. Med. Bull. 94, (2010): 165). Briefly, tECM-GeIMA hydrogels (round disk: 8 mm (diameter)×1 mm (thickness)) were prepared and incubated in 1 mL of 1×PBS in 24-well plates. As a control, the same volume of GelMA hydrogel without tECM was also prepared. At 24-hour intervals over the course of 12 days, a 0.5-mL aliquot was collected, and the incubation solution was subsequently replenished with an equal volume of PBS. The protein content of collected samples was assessed using the bicinchoninic acid (BCA) assay to determine the amount of tECM released. The daily release and cumulative release of tECM protein profile were determined using the following formulas:
Daily protein release profile:

$$P t ( \% ) = \frac{C t \times V}{W e} \times 100$$

Cumulative protein release profile:

$$Cumulative percentage release ( \% ) = \frac{C tV {\sum }_{i = 1}^{i = n - 1} C i V i}{W ( e )} \times 100 \%$$

Pt: daily release, percentage release at time t (day); Ct: concentration of proteins at time t (day);
V: the volume of incubation solution; W(e): total mass (pg) of the encapsulated proteins.

QHM elastomer was fabricated as previously described, with modifications (D. F. E. Ker et al., Adv. Funct. Mater. 28, (2018): 1707107). Due to local humid environment, water molecules in the air may hinder the reaction between the hydroxyl group of Q and the isocyanate groups of H or the anhydride carbonyl group of M. This can compromise the mechanical properties of the QHM elastomer. Therefore, a drying step was performed to remove the absorbed water in Q. For drying Q, 5 g of MgSO₄ (as a drying agent) was added to 30 mL of Q, and the resulting mixture was vortexed and stored in an oven at 90 TC before being filtered with a filter paper (DotMed). The freshly dried Q was mixed with H and M in a 50-mL conical tube at a molar ratio of 1:1.5:0.5, respectively, prior to vigorous vortexing for 1.5 minutes. Subsequently, the QHM mixture was degassed in a vacuum desiccating chamber for 2 minutes and transferred into the appropriate mold. The mold with QHM prepolymer was degassed for an additional 30 minutes and then placed in a pressure pot chamber at 40 psi under a nitrogen atmosphere overnight. The following day, QHM elastomer samples were released from their molds, sanded using a sandpaper (Eagle Brand, Korea), and heat-cured between 90° C. for 5 hours in a heating oven.

### Example 2. Robust Interfacial Bonding in the Core-Shell Structured Hybrid Scaffold Graft Material

The bonding integrity of the hybrid constructed HyS is essential for its in vivo application. To investigate the attachment strength of the core (QHM elastomer) and shell (GeIMA hydrogel) layers of HyS, quantitative assessment in the form of a 90-degree peeling test and qualitative assessments including scanning electron microscopy (SEM) and in vivo mouse subcutaneous histological assessments were used (FIGS. 11-13).

To quantitatively measure the interfacial bonding strength between the GeIMA hydrogel layer (thickness, 1 mm) and the QHM elastomer, bonded (photocrosslinked) and nonbonded (nonphotocrosslinked) HyS were subjected to 90-degree peel testing with a peel rate of 50 mm/min (FIG. 11). Briefly, the QHM elastomer was secured onto a rigid bottom substrate (stage platform of the peel test grip) while the GelMA hydrogel was attached to a thin supporting substrate (GORILLA® tape) at the top, which minimized undesired hydrogel damage and elongation during testing (FIG. 11). Subsequently, the interfacial bonding strength was assessed by force-displacement profile and failure mode analysis (FIG. 11). Data showed that bonded HyS yielded significantly higher peeling force and peak load compared to nonbonded HyS (FIG. 11). Additionally, in bonded HyS, failure occurred within the GelMA hydrogel bulk rather than at the GeIMA hydrogel-QHM interface as shown by the residual layer of GelMA hydrogel remaining on the QHM elastomer (FIG. 11). In contrast, failure of nonbonded HyS occurred at the GelMA hydrogel-QHM interface rather than within the GelMA hydrogel bulk, with the hydrogel being easily peeled off from the QHM elastomer surface (FIG. 11).

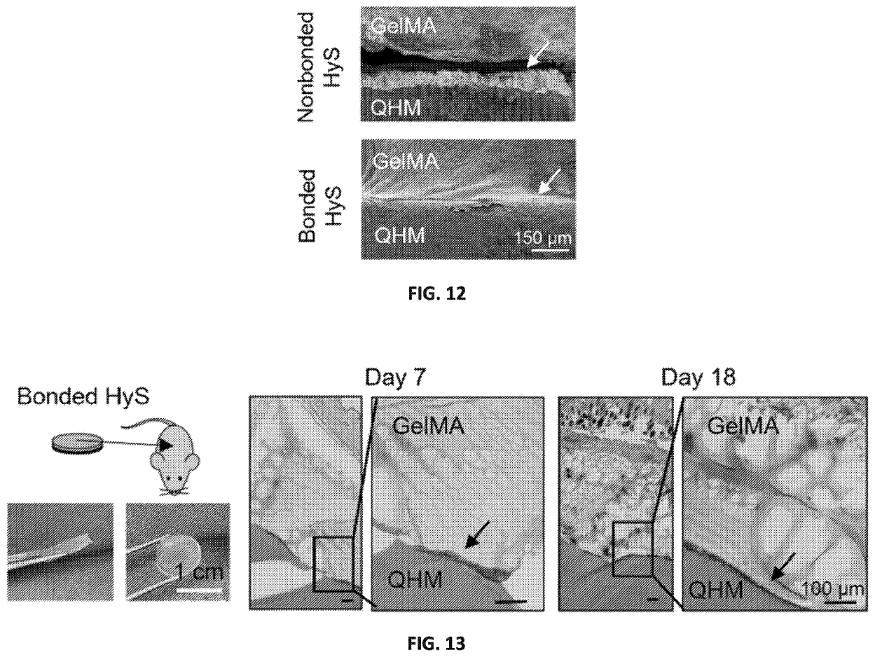

To qualitatively assess interfacial bonding strength, scanning electron microscopy (SEM) and in vivo mouse subcutaneous implantation studies (with Hematoxylin and Eosin (H&E) staining) were performed on HyS (FIGS. 12 and 13). SEM analysis was performed to characterize the interfacial bonding between GelMA coating and elastomer core of HyS. HyS with (bonded HyS) or without (nonbonded) photocrosslinking treatment were included for SEM analysis. The interfaces between GelMA hydrogel and QHM elastomer were examined by SEM (Hitachi SU8010). The specimens (4 mm (width)×4 mm (length)×2 mm (thickness)) were prepared and lyophilized before SEM analysis. The cross-sections of the samples were examined after sputter-coating with a thin layer of gold, and elemental analysis was performed using a SEM.

Representative SEM images showed that GeIMA hydrogel firmly attached to the QHM elastomer for bonded HyS, whereas nonbonded HyS showed a clear delamination with little-to-no residual GelMA hydrogel on the QHM elastomer (FIG. 12). Furthermore, bonded HyS samples were subcutaneously implanted in mice. H&E staining showed that bonded HyS maintained robust attachment between the GeIMA hydrogel and QHM elastomer at 7- and 18-days postgrafting (FIG. 13).

Together, the quantitative peel testing results as well as qualitative SEM and in vivo mouse subcutaneous studies demonstrated that bonded HyS using benzophenone mediated photocrosslinking treatment exhibits strong interfacial attachment strength between its hydrogel and elastomer layers.

### Example 3. Robust Tensile Properties and Suture Retention Values of the Hybrid Scaffold Graft Material

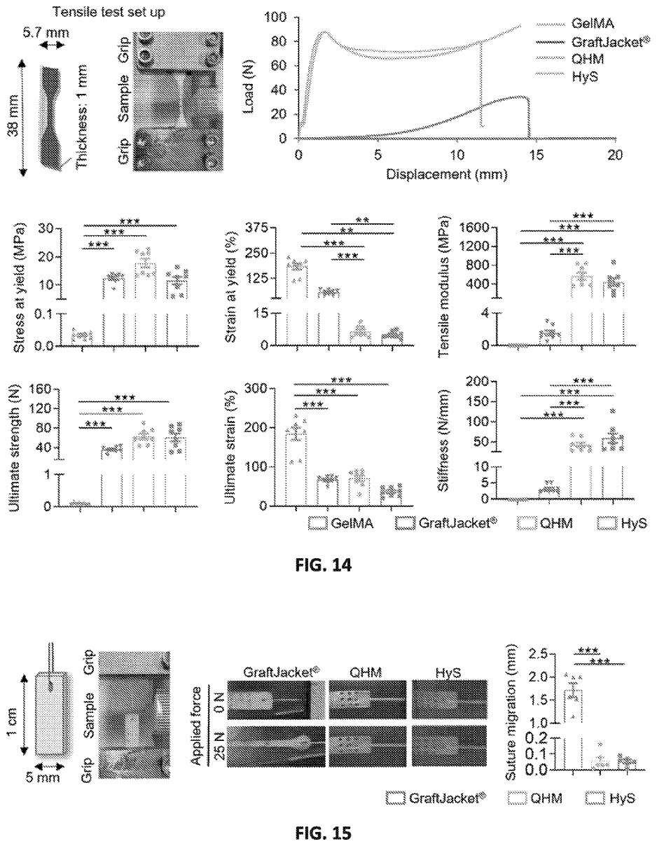

Prior work showed that the QHM elastomer, the core material of the provided hybrid scaffold graft material, exhibits human SSPT-like tensile attributes and excellent suture retention (D. F. E. Ker et al., Adv. Funct. Mater. 28, (2018): 1707107). In this example, tensile and suture retention tests were performed to ensure that the introduction of benzophenone mediated photocrosslinking treatment for HyS fabrication does not adversely affect the tensile properties and allows the provided graft material to robustly sustain surgical repair (FIGS. 14 and 15).

To assess whether HyS could sustain physiologically relevant mechanical loads during tendon functional movement, tensile testing was performed on GeIMA hydrogel, QHM elastomer, commercially available acellular dermal matrix (acellular dermal matrix (ADM); GRAFTJACKET®, as a clinical scaffold control), and HyS (FIG. 14). For the tensile test, samples were fabricated in a modified type IV shape following the guidelines in the American Society for Testing and Materials (ASTM) method D638-10. A horizontal mechanical tester (Admet) equipped with a 500-lb load cell was used together with MTestQuattro software (Admet, Version 6.00.05) to acquire tensile data. Representative force-displacement profiles showed that the QHM elastomer and HyS similarly exhibited an initial steep increase in load-displacement followed by sustained plastic behavior prior to failure, whereas ADM clinical control exhibited a gradual increase in load-displacement followed by failure after sustained deformation (FIG. 14). The force-displacement profile of GelMA hydrogel was not evident due to failure at low load (FIG. 14). As with force-displacement profiles, the QHM elastomer and HyS exhibited similar tensile properties in terms of stress at yield, strain at yield, tensile modulus, ultimate strength, ultimate strain, and stiffness (FIG. 14). Most notably, the QHM elastomer and HyS exhibited human SSPT tensile moduli (approximately 500-600 MPa) (D. F. E. Ker et al., Adv. Funct. Mater. 28, (2018): 1707107) which were 400-fold higher than ADM and 25,000-fold higher than GelMA hydrogel. Interestingly, ADM showed similar stress at yield (11.9 f 0.5 MPa) as HyS and QHM elastomer (12.5±0.3 MPa and 17.65 f 1.1 MPa, respectively) but higher strain at yield (ADM: 56.3±2.4%, HyS: 5.05±0.2%, and QHM elastomer: 5.3±0.4%), resulting in a lower tensile modulus and stiffness than the QHM elastomer and HyS (FIG. 14). Thus, tensile testing showed that the QHM elastomer and HyS exhibited human SSPT-like tensile attributes, which were superior to those exhibited by ADM and GelMA hydrogel.

To assess whether HyS could sustain surgical repair (since rotator cuff repairs frequently fail by tendon pull-through of suture), a suture retention test was performed on rectangular-shaped ADM, QHM elastomer, and HyS (FIG. 15). For the suture retention test, a horizontal mechanical tester (Admet) equipped with a 100-lb load cell was used together with MTestQuattro software (Admet, Version 6.00.05) to acquire data. When 25 N force was applied, no obvious suture migration (less than 0.1 mm) or scaffold deformation was observed on either the QHM elastomer or HyS, while ADM exhibited significant elongation and approximately 1.72 mm suture migration (FIG. 15). The results showed that HyS maintained its shape and prevented suture pull-through effect under 25 N, suggesting HyS exhibited superior suture retention properties.

Together, these results demonstrate that HyS exhibited human SSPT-like tensile properties and excellent suture retention properties superior to those of a leading commercial scaffold ADM, suggesting that HyS has clinically favor biomechanical attributes vital to rotator cuff repair stability.

### Example 4. Strong Proliferation and In Vitro Tenogenic Differentiation of hASCs Encapsulated in tECM-HyS

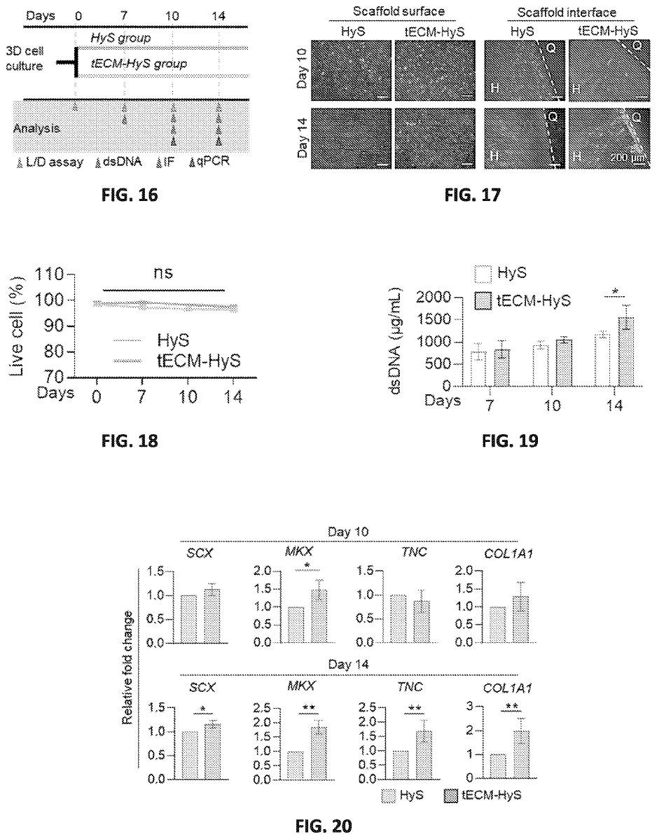

Since benzophenone mediated photocrosslinking treatment was performed during tECM-HyS fabrication, such treatment may potentially influence the bioactivity of tECM within HyS GelMA layer. Therefore, to evaluate the biocompatibility and tenogenic bioactivity of tECM-HyS, hASCs were encapsulated into the HyS, and in vitro studies on cell viability, proliferation, and tenogenic differentiation were performed (FIG. 16). hASCs were encapsulated in the gel layer of HyS or tECM-HyS, and a set of experiments were performed to characterize cell viability, proliferation, and tenogenic differentiation. In brief, hASCs (passage 2-5, 4 million/mL) were mixed with GelMA prepolymer containing either 20% (v/v) tECM (final concentration in gel: 0.6 mg/mL) as tECM-GeIMA group or 20% (v/v) PBS as HyS group and hybrid constructs were formed by UV irradiation (365 nm, 25 mW/cm², 90 s). Different groups of scaffolds were cultured in growth medium (DMEM-high glucose (Thermo Fisher), 10% (v/v) FBS, 1% (v/v) P/S, and 50 ng/mL ascorbic acid (Santa Cruz). On days 7 and 14, live/dead (Invitrogen) and dsDNA (Quant-iT PicoGreen dsDNA Reagent, Invitrogen) assays were performed to determine cell viability and dsDNA content, respectively.

The cell viability of encapsulated hASCs was assessed up to 14 days using a live/dead assay (FIGS. 17 and 18). Fluorescence microscopy was utilized to visualize the distribution of live and dead cells in the middle portion of the gel as well as the interface between the gel and the QHM elastomer. From day 7 to day 14, high cell viability (more than 90%) was observed in both HyS and tECM-HyS, indicating that HyS and tECM-HyS were both cytocompatible. Cell proliferation was assessed by dsDNA assay (FIG. 19). After 14 days of culture, a significantly higher amount of dsDNA was measured in the tECM-HyS group than that in the HyS group (FIG. 19), suggesting that tECM-HyS enhanced cell proliferation compared to HyS.

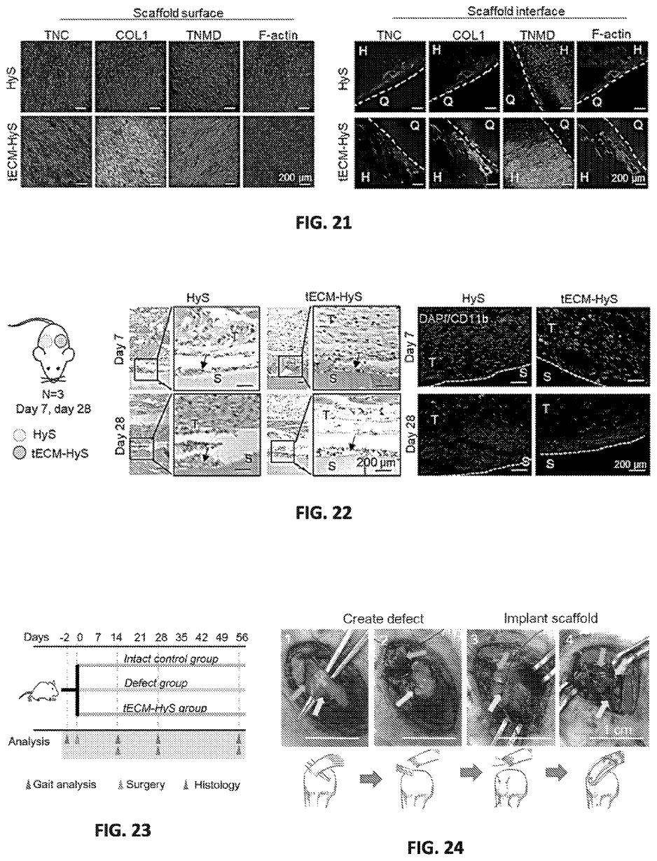

Tenogenic differentiation was assessed using qRT-PCR (SCX, MKX, TNC, and COL1A1) and fluorescence staining (TNC, COL1 TNMD, and F-actin) for tenogenesis-associated markers (FIGS. 20 and 21). For 2D cell culture, cells were fixed with 4% (w/v) paraformaldehyde (PFA) at room temperature for 30 minutes and permeabilized with either Triton-X100 (0.1% w/v in PBS) or saponin (0.5% w/v in PBS) at room temperature for 20 minutes. For 3D cell culture, hydrogels were fixed with 4% (w/v) PFA at room temperature for 1 hour and permeabilized with Triton-X100 (0.10% w/v in PBS) at room temperature for 30 minutes. The sample was then incubated with 10% (v/v) donkey serum (Sigma-Aldrich) to block nonspecific bonding, followed by incubation overnight at 4° C. with primary antibodies including rabbit anti-SCX (1:200; Abcam, ab58655), mouse anti-TNC (1:500, ab3970), rabbit anti-COL1 (1:250; NOVUS, NBP1-89912), rabbit anti-TNMD (1:500; Abcam, ab203676). Alexa Fluor 488 donkey anti-rabbit (1: 500; Abeam, A21206) or Alexa Fluor 647 donkey anti-mouse (1:500; Abeam, A21571) was used as a secondary label and incubated with samples for 1 hour at room temperature. Cells were nuclear-counterstained with Hoechst (1:1000; Thermo Fisher, 62249) for 15 minutes and imaged using fluorescence microscope (Olympus IX83-ZDC Inverted Microscope) for 2D cell culture or confocal microscope (Leica TCS, SP8 Inverted confocal microscope) for 3D cell culture. qPCR was performed to investigate the tenogenic gene expression of hASCs in tECM-HyS. At designated time points, total RNA was isolated by Trizal (Invitrogen) and RNA extraction kit (New England Biolabs) and cDNA was synthesized with a reverse transcription PCR kit (New England Biolabs) as previously described (D. Wang et al., FASEB J. 34, (2020): 8172). qPCR was performed using Luna Universal Probe qPCR Master Mix (New England Biolabs) on a qPCR system (Applied Biosystem, Life Technology) and the A 6-FAM dye-labeled TaqMan MGF probe (Thermo Fisher) was used to identify tenogenesis marker genes, including scleraxis (SCX; Hs03054634_g1), tenascin C (TNC; Hs01115665_ml), type I collagen (COL1A1; Hs00164004_ml), and mohawk (MKX; Hs00543190_ml). Relative fold change gene expression was calculated using the ΔΔCT method and normalized to FBS control group.

After 10 and 14 days of culture, significantly increased expression of SCX, MKX, TNC, and COL1A1 was observed in the tECM-HyS relative to the HyS control groups (FIG. 20). Consistently, fluorescence staining results showed that enhanced expression of all tenogenesis associated markers (COL1, TNC, and TNMD) in the tECM-HyS group relative to the HyS group (FIG. 21).

Taken together, the findings demonstrated that the benzophenone photocrosslinking procedure to form tECM-HyS hybrid did not affect its bioactivity. tECM-HyS exhibited excellent cytocompatibility, but also markedly promoted hASC proliferation and tenogenic differentiation in vitro.

### Example 5. Biocompatibility of tECM-HvS in a Mouse Subcutaneous Implantation Model

To examine the biocompatibility of tECM-HyS in vivo, tECM-HyS and HyS were processed in the form of discs and implanted subcutaneously into the wild-type mice for histological (H&E and IHC) evaluation at designated timepoints (FIG. 22). Each mouse was implanted with one HyS and one tECM-HyS scaffold (a round disk shape; 8 mm (diameter)×2 mm (thickness)) and euthanized at designed timepoints (post-implantation days 7 and 28) for further histological examination. At day 7, infiltration of a small number of cells positive for CD11b+ (a pan-myeloid marker) were observed at the surface area of the tECM-HyS. At day 28, both HyS and tECM-HyS were surrounded by a thin fibrous capsule. There was no obvious evidence of an overexuberant inflammatory response in both implantation groups. Thus, the histology results suggested that both HyS and tECM-HyS were biocompatible.

### Example 6. Promotion of Shoulder Function Restoration by tECM-HvS in a Rat Full-Thickness Rotator Cuff Tendon Defect Model

Tendon function recovery is a crucial demand of patients with tendon injuries (Y. Liu et al., J Orthop Translat 26, (2012): 31). However, most preclinical studies only focused on biological and mechanical assessments while the functional evaluation was often neglected (Y.

Liu et al., J Orthop Translat 26, (2012): 31). In light of this, a rat massive rotator cuff tendon defect model as previously described (J. Beck, D. Evans, P. M. Tonino, S. Yong, & J. J. Callaci, Am. J. Sports Med. 40, (2012): 2037) was used with Sprague-Dawley rats assigned to three groups: (1) intact control group, (2) defect only group, and (3) tECM-HyS group (FIG. 23). Tendon repair efficacy of tECM-HyS was evaluated using macroscopic, histological, micro-CT, and gait analyses at designated time points after the surgery (FIGS. 23-29). A total of 36 Sprague-Dawley rats (gender: male; average age: 12 weeks; weight, 250 g; LASEC, CUHK) were assigned into three groups: (1) the intact control group, (2) defect only group, and (3) tECM-HyS implantation group. For the intact control group, no defect or treatment was performed. For the defect only group, no treatment was applied after creating the tendon defects and the wound was closed immediately after the surgery. For the tECM-HyS implantation group, a 5.0 Prolene suture (Ethicon) was passed through the SSPT and firmly secured on to the tECM-HyS (rectangle shape; 2 mm (width)×3 mm (length)×1 mm (thickness)) with completion of a locking stitch using a modified Mason-Allen technique (N. Bhalerao & S. Pagdal, Int. J. Orthop. Sci. 5, (2019): 13; H. M. Klinger, H. Steckel, G. Spahn, G. H. Buchhorn & M. H. Baums, Clin. Biomech. (Bristol, Avon) 22, (2007): 106). Another suture end was passed through the bone tunnels, which were drilled at the greater tuberosity using a 0.5 mm drill, and then tied with the tECM-HyS approximated to the anatomic footprint on the greater tuberosity (N. Bhalerao & S. Pagdal, Int. J. Orthop. Sci. 5, (2019): 13).

Although in this animal model, tendon defects were only made on rat SSPT, bone tunnels were created to secure the scaffolds using sutures. Therefore, micro-CT was performed to evaluate the bone density and new bone formation at tendon insertion and the bone tunnel sites. Each sample was scanned with micro-CT (Scanco Medical). The resolution was set to 19 μm per voxels and 1024×1024 pixels. The images were thresholded to distinguish bone voxels using a standard threshold for each sample. A low pass Gaussian filter (Sigma=1.2, Support=2) was used for 3D reconstruction. The scanning data was analyzed by Scanco Medical [CT 35 software (Scanco Medical). After thresholding, the bone volume fraction (BV/TV), bone mineral density of total volume (BMD of TV), bone mineral density of bone volume (BMD of BV), trabecular number (Tb·N), trabecular thickness (Tb,Th), and trabecular separation (Tb·Sp) were calculated.

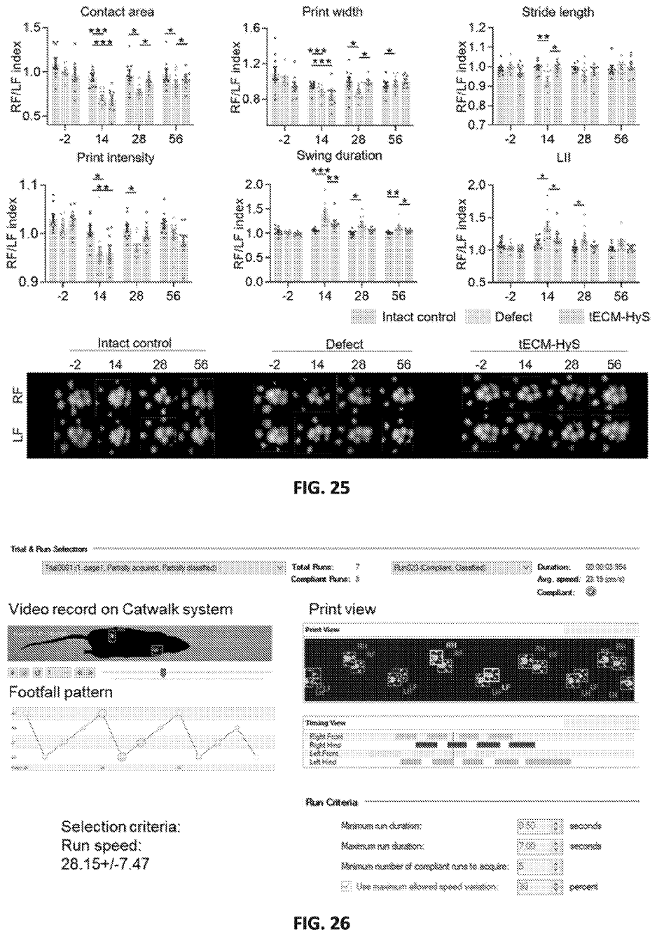

Gait parameters and gait patterns, such as paw contact area, print width, stride length, paw print intensity, swing duration, and limb idleness index (LII), were examined using a rodent CatWalk system (FIGS. 25 and 26) (Y. Liu et al., J Orthop Translat 26, (2012): 31; S. C. Fu, Y. C. Cheuk, L. K. Hung & K. M. Chan, Osteoarthritis Cartilage 20, (2012): 1409). Gait analysis was conducted 2 days before surgery as well as 2 weeks, 4 weeks, and 8 weeks after surgery. The automated Catwalk system (Catwalk XT 9.0; Noldus) employed a motorized platform microscope (Leica) and ImagePro image premier analysis software (Media Cybernetics). The rats were pretrained to cross the walkway daily for 2 days before the surgery and data were collected to assess the presurgery gait performance. At 2, 4 and 8 weeks postsurgery, the walking behaviors of the rats were recorded. During the data collection, the walkway was set as a horizontal platform, the camera was set at 60 cm from the walkway, and the entire run was recorded by a video camera. Recorded runs with a steady walking speed were accepted as compliant runs for paw-print autoclassification of left forelimb (LF), right forelimb (RF), left hind limb (LH) and right hind limb (RH) by the built-in software. The footprint intensity, footprint area, print width, stride length, and LII were calculated (Y. Liu et al., J. Orthop. Translat. 26, (2021): 31; S. C. Fu, Y. C. Cheuk, L. K. Hung & K. M. Chan, Osteoarthritis Cartilage 20, (2012): 1409).

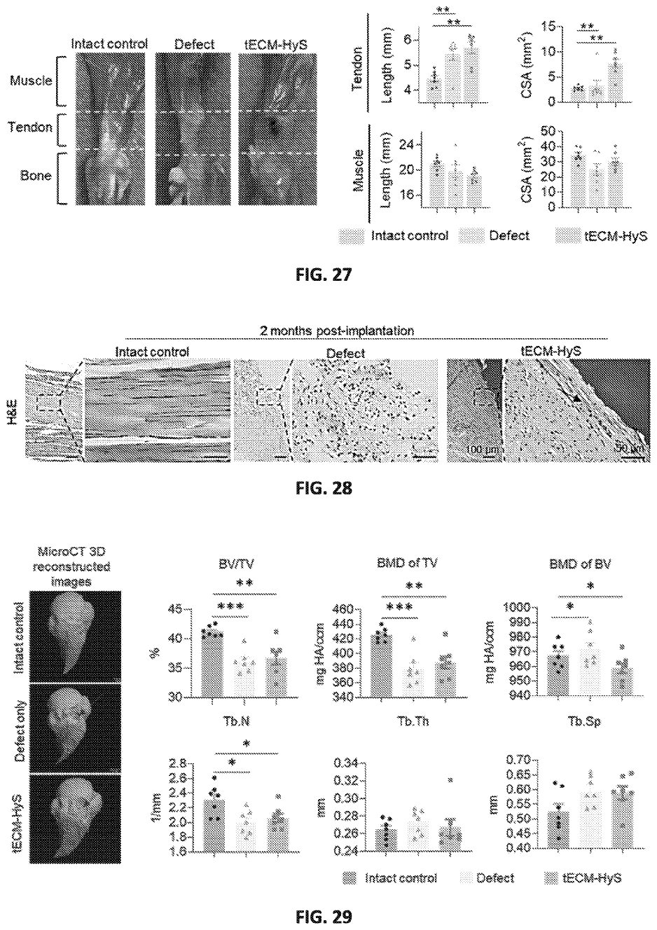

Usually, the impaired shoulder function and pain due to tendon tear prompted the rats to avoid bearing weight on the injured paws, resulting in a lower contact intensity, print width, and print intensity, a longer swing duration, and a higher LII than the uninjured contralateral paw (Y. Xu et al. Neurosci. Bull. 35, (2019): 401; S. M. Perry, C. L. Getz & L. J. Soslowsky, J. Shoulder Elbow Surg. 18, (2009): 296). Results showed that at an early time point after the surgery, i.e., day 14, significant differences in gait parameters (such as the contact area, print width, stride length, and print intensity) were observed between the tECM-HyS and intact control groups (FIG. 25). On days 28 and 56, comparable gait parameters indicating similar gait performance were observed between the tECM-HyS and intact control groups (FIG. 25). Although some studies suggested that spontaneous tendon healing could be achieved in rats (J. H. Choi, I. K. Shim, M. J. Shin, Y. N. Lee & K. H. Koh, PLoS One 17, (2022): e0266030), the decreased values of contact area, print width, and print intensity and the increased values of swing duration and LII indicated that the shoulder function in the defect only group was not recovered after surgery even after 56 days (FIG. 25). At 2 months post-operatively, the regenerated SSPTs were observed macroscopically, histologically, and radiologically (FIGS. 27-29). These results indicated that neotendon tissue was formed in the tECM-HyS group with no obvious inflammatory response (FIGS. 27 and 28). However, the implantation of tECM-HyS did not help with the healing of bone-tunnel created in the rat rotator cuff injury model (FIG. 29). Using a massive rotator cuff tendon defect model in rat, results therefore demonstrated that tECM-HyS improved the quality of tendon healing and augmented shoulder functional restoration as evidenced by the gait analysis.

### Example 7. Superior Tendon Regeneration with Robust Biomechanical Properties Achieved with tECM-HyS in a Rabbit Massive Rotator Cuff Tendon Defect Model

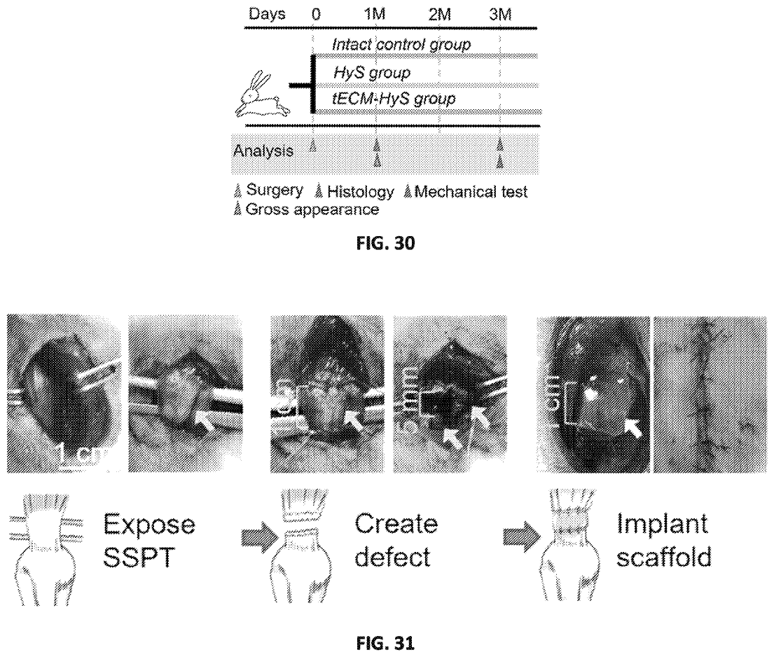

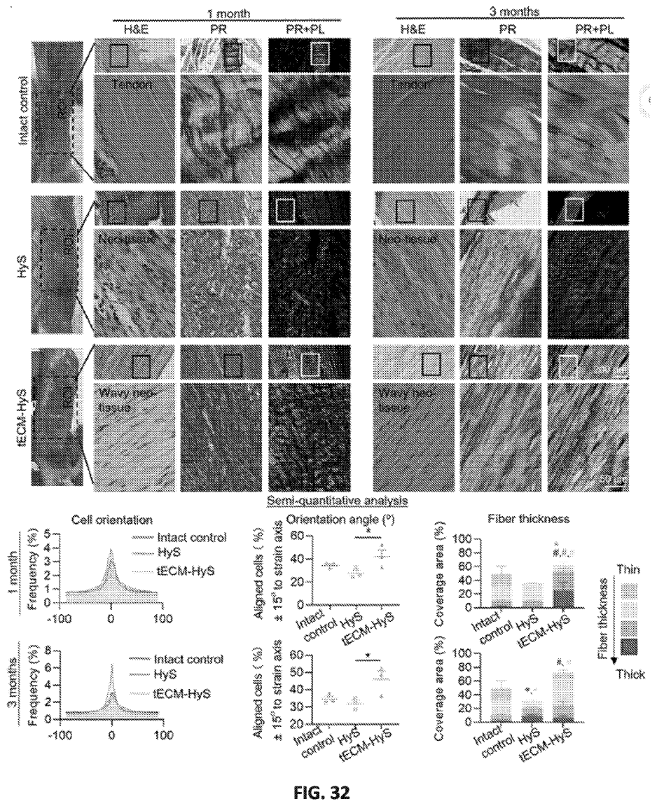

To investigate the healing efficacy of tECM-HyS for tendon tears, a rabbit massive rotator cuff tendon defect model was established. A rabbit model was selected to test because rabbits are larger than rats, therefore facilitating surgical models and techniques as well as providing greater accuracy and reproducibility (G. Depres-Tremblay et al., J. Shoulder Elbow Surg. 25, (2016): 2078). Additionally, rabbits can generate stronger shoulder force to better challenge the tendon healing capacity of our tECM-HyS (V. Burgio et al., Processes 10, (2022): 485). To determine whether tECM-HyS promotes tendon healing efficacy, a 1-cm length, full-thickness tendon defect was created on rabbit SSPT and implanted with HyS or tECM-HyS (FIGS. 30 and 31). The contralateral shoulder was used as an intact control group. Additionally, a mock surgery on cadaver rabbits was performed to evaluate the initial mechanical properties of HyS augmented tendons. To evaluate tissue healing, macroscopic, histological (H&E, picrosirius red) and biomechanical analysis were performed to assess tendon gross appearance, neotissue cell alignment, collagen fiber orientation, density, and maturation (FIGS. 32-34).

More specifically, a total of 40 New Zealand White rabbits (gender, male or female; average age, 16 weeks; weight, 3.0-4.0 kg; LASEC, CUHK) were randomly assigned into 3 groups: (1) intact control group, (2) HyS implantation group, and (3) tECM-HyS implantation group. All animals underwent an index procedure in which either the left or right SSPT was randomly detached, leaving the contralateral intact shoulder as an intact control group. Under anesthesia, a full-thickness tendon defect 5 mm in length was made on the rabbit SSPT (Z. Zheng et al., Acta Biomater 51, (2017): 317). Subsequently, a tECM-HyS or HyS scaffold (rectangle shape; 10 mm (width)×10 mm (length)×2 mm (thickness)) was implanted and connected to the two ends of the defect tendons by sutures using a modified Masson-Allen technique (H. M. Klinger, H. Steckel, G. Spahn, G. H. Buchhorn & M. H. Baums, Clin. Biomech. (Bristol, Avon) 22, (2007): 106). The rabbits were euthanized at the designated time points, and the entire supraspinatus muscle and tendon unit was harvested for further analysis.

At designated time points, the harvested tendon specimens were immediately fixed in 4% (w/v) PFA for 48 hours, dehydrated, and embedded in paraffin blocks. Histological sections (7 mm) were prepared using a microtome (Leica, RM2235) and subsequently stained with H&E, Picrosirius red, and Masson's Trichrome. The stained sections were photographed digitally under a microscope (Nikon Ni-U Eclipse Upright Microscope) or polarizing microscopy (Nikon Ni-U Eclipse Upright Microscope equipped with D-SA Analyzer Slide for Simple Polarization).

To qualitatively and quantitatively assess collagen during tendon healing, combined use of picrosirius red staining, circularly polarized light, and image-analysis software, was performed to identify collagen fibers and assess collagen content and spatial distribution. The area of collagen fibers as a function of their color hue was quantified from tissue sections stained with Picrosirius red and imaged with polarized light microscopy. The color hue corresponds to relative fiber thickness from thin green fibers to increasingly thick yellow, orange, and red fibers.

Images (400× magnifications) observed using polarized light microscopy were converted from 24-bit RGB (red, green, and blue) to 8-bit HSB (hue, saturation, brightness) stack using ImageJ software (NIH). By using threshold function, color intensity was defined as 2-9 and 230-256 for red, 10-38 orange, 39-51 yellow and 52-128 green. The hue intensity for 129-229 was defined as nonbirefringent collagen and total hue range considered as collagen pixel percentage. The collagen content for each color component was calculated as follows:

$$Collagen fraction cover area ( \% ) = \frac{Cover area of color fraction}{Total area} \times 100 \%$$

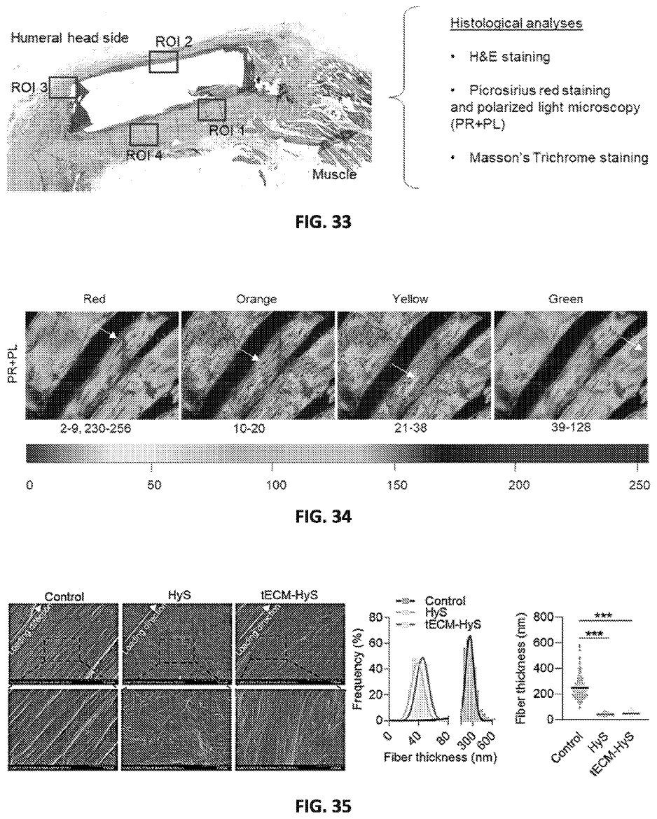

At 1 month after surgery, no obvious symptoms of severe inflammation were shown in either HyS or tECM-HyS groups based on the gross appearance of tendons (FIG. 32). H&E staining images showed that both the HyS and tECM-HyS groups exhibited increased cellularity relative to the intact control group. Semiquantitative cellular orientation analysis showed that cells were more aligned in tECM-HyS than in HyS group. Picrosirius red staining examined by polarized light microscopy (FIGS. 33 and 34) was used to assess the birefringent nature of anisotropic molecules, characteristic of tendon ECM, with scar tissue generally having less birefringence (P. B. Voleti, M. R. Buckley & L. J. Soslowsky, Annu. Rev. Biomed. Eng. 14, (2012): 47). Semi-quantitative results from Picrosirius red staining showed that birefringence of the HyS was less than the tECM-HyS group and the majority of fibers in HyS group were yellow while in tECM-HyS group was orange-to-red, indicating that the neotissue in tECM-HyS group were wavy and aligned while the HyS group was loose and disorganized (FIG. 32). At 3 months after surgery, decreased cellularity was observed in both HyS and tECM-HyS groups and better cellular alignment was observed in tECM-HyS group compared to HyS group (FIG. 32). The images of picrosirius red staining with polarized microscopy showed that the birefringence in HyS was significantly less than the tECM-HyS group, suggesting that the amount and fiber size of deposited collagen fibers was lower for the HyS group compared to control and tECM-HyS group (FIG. 32).

Data showed enhanced collagen synthesis and better ECM organization in tECM-HyS group which was similar to control group at 3 month after surgery (FIG. 32). Thus, histological analysis demonstrated that tECM-HyS exhibited improved rotator cuff tendon healing at both 1 month and 3 months post-implantation relative to HyS group that lacking tECM supplementation.

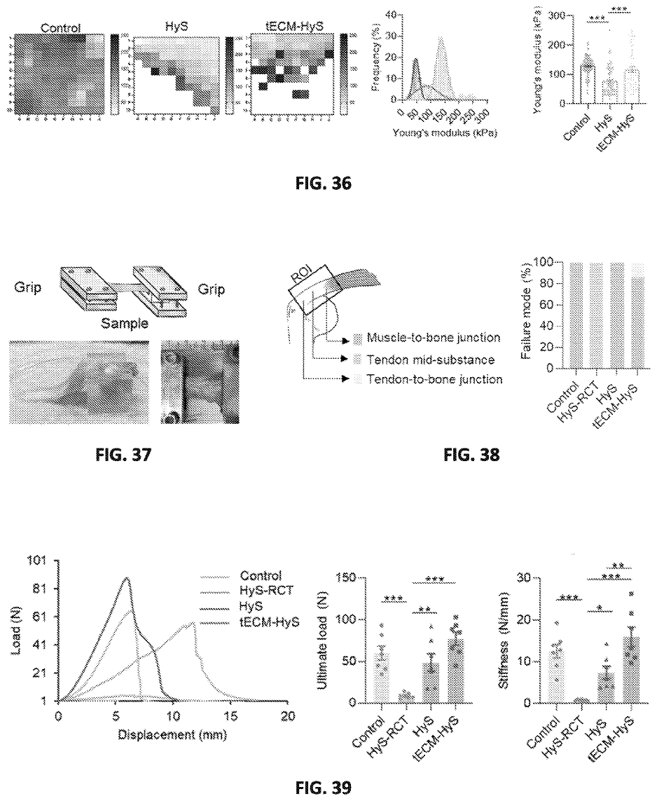

Successful tendon repair also requires recovery of tissue strength, which is important for the tendon to fulfill its functionality and avoid retear occurrence, especially in cases of large tendon tears. Therefore, to assess rotator cuff shoulder function, biomechanical testing of the SSPT-humeral bone complex for intact control group, HyS repaired rabbit cadaver tendon group (HyS-RCT; mock surgery that HyS was implanted in a rabbit cadaver model), HyS (at 3 month after implantation), and tECM-HyS (at 3 month after implantation) was performed (FIGS. 35-39).

The tensile test was performed to evaluate the biomechanical properties of regenerative tendon as previously described (D. F. E. Ker et al., Adv. Funct. Mater. 28, (2018): 1707107). The tissue samples were harvested, wrapped in saline-soaked gauze, sealed in plastic bags, and immediately frozen at −20° C. Before tensile testing at room temperature, the tendons were thawed on ice and slowly warmed to room temperature. A horizontal mechanical tester (Admet) equipped with 500-lb load cells was used together with MTestQuattro Software (Admet, Version 6.00.05) to acquire tensile data. Samples were secured with the tendon end gripped via sandpaper and the humeral head bone mounted in a custom-made block as shown in FIG. 37. Sample were preloaded to 1 N and subsequently uniaxial loaded at a rate of 0.15 mm/s, which corresponds to a strain rate of about 1% per second, until failure. Failure mode was recorded during testing. Ultimate load was defined as the highest load at which the construct failed. Stiffness was defined as the change in tissue sample length in relation to the applied force.

In the HyS-RCT group, all repaired tendons failed at the tendon mid-substance due to suture pullout. In both the intact control and HyS groups, failure sites were shifted toward the musculotendinous junction. In the tECM-HyS group, ruptures at both tendon-to-bone and muscle-to-bone junctions were observed (FIGS. 35-39). There were no differences in failure mode among the intact control, HyS, and tECM-HyS groups. Based on the tensile tests (FIGS. 35-39), the tECM-HyS group achieved comparable maximum load and stiffness (77.47±2.89 N; 15.94±0.13 N/mm) with the intact control group (54.29±3.1 N; 10.8±0.61 N/mm), and significantly higher than the HyS-RCT group (9.12±0.49 N; 0.82±0.04 N/mm). Furthermore, although HyS group showed comparable maximum load (48.63±4.0 N) with the intact control and tECM-HyS group, its stiffness (7.40±0.57 N/mm) was significantly lower tECM-HyS group.

Taken together, these results demonstrate that tECM-HyS enhanced rotator cuff tendon regeneration and biomechanical features in a rabbit massive tendon defect (>1 cm) model.

### Example 8. Pro-Tenogenesis Bioactivity of Enzymatically Digested tECM

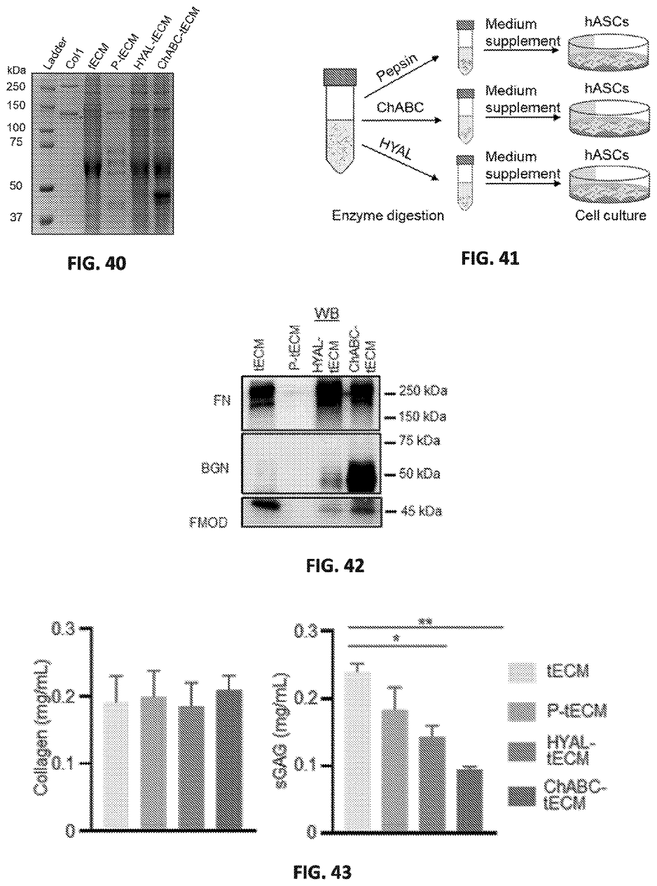

Previous work revealed that urea-extracted tECM contains a number of non-collagenous ECM components, including fibronectin and other low-to-medium molecular weight components (<100 kDa), such as decorin, biglycan, and fibromodulin (G. Yang et al., Biomaterials 34, (2013): 9295; D. Wang et al., FASEB J. 34, (2020): 8172). As illustrated in FIG. 41, to assess the nature of the molecular moieties responsible for tECM pro-tenogenesis bioactivity, tECM was treated separately with: (1) pepsin (0.1 mg/mL, pH=2, 6 hours), a non-specific protease for most proteins except for native, triple helical collagens (Z. Fu et al., Biol. Chem. 402, (2021): 861); (2) chondroitinase (ChABC) (0.02 mg/mL, pH=8, 6 hours), which mostly digests chondroitin sulfate (CS) (W. Wang, J. Wang & F. Li, Adv. Exp. Med. Biol. 925, (2017): 75); and (3) hyaluronidase (HYAL) (0.14 mg/mL, pH=5.3, 6 hours), which mostly digests hyaluronic acid (HA) (W. Wang, J. Wang & F. Li, Adv. Exp. Med. Biol. 925, (2017): 75). Specifically, pepsin (Worthington, USA, 9001-75-6) was dissolved in 1 N HCl at 1 mg/mL and mixed into tECM (1 mg/mL) at a volumetric ratio of 1:10, with pH adjusted to 2 for optimal efficiency, and incubated at room temperature for 6 hours with continuous agitation (J. F. Collins & R. Fine, Biochim. Biophys. Acta 657, (1981): 295; Z. Fu et al., Biol. Chem. 402, (2021): 861). Hyaluronidase (Worthington, LS002592) was dissolved in Tris buffer (1 M in dH₂O; Sigma-Aldrich, USA) at 1 mg/mL and mixed with tECM (1 mg/mL) at a volumetric ratio of 4:25, with pH adjusted to 5.3 for optimal efficiency, and incubated at 37° C. for 6 hours with continuous agitation (W. Wang, J. Wang & F. Li, Adv. Exp. Med. Biol. 925, (2017): 75). Chondroitinase (Sigma-Aldrich, 9024-13-9) was dissolved in Tris buffer at 1 mg/mL and mixed into tECM (1 mg/mL) at a volumetric ratio of 3:125, with pH adjusted to 8 for optimal efficiency, and incubated at 37 C for 6 hours with continuous agitation (W. Wang, J. Wang & F. Li, Adv. Exp. Med. Biol. 925, (2017): 75). All enzyme activities were completely blocked with a neutral pH and in a serum-containing environment.

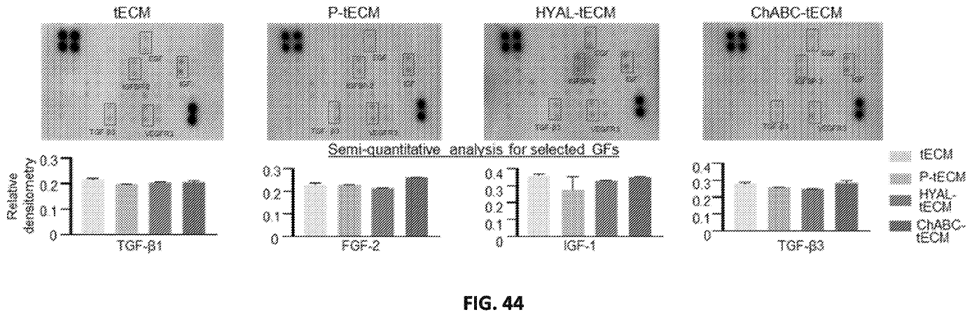

Subsequently, the protein profile of the enzymatically treated tECM preparations was analyzed by sodium dodecyl sulfate-polyacrylamide gel electrophoresis (SDS-PAGE) (FIG. 40). Briefly, protein samples of the same concentration were loaded onto the gel and separated by electrophoresis (90 V for stacking gel and 120 V for resolving gel). The SDS-PAGE gel was fixed and stained with Coomassie brilliant blue (Thermo Fisher Scientific). The background was then distained by a solution of 30% (v/v) methanol and 10% (v/v) acidic acid in dH₂O until the protein band was sufficiently observable. The results showed that the P-tECM-treated group contained mostly collagen chains, consistent with α1 and α2 chains, as well as dimeric P-bands, a pattern similar to that of the commercial Coll preparation. Other protein bands in P-tECM were detected between 75 kDa and 37 kDa. In contrast, the protein patterns of HYAL-tECM and ChABC-tECM were similar to that of untreated tECM (FIG. 40) (W. B. LaRiviere et al., J. Vis. Exp. 168, (2021): 10.3791/62319). Western blot analysis demonstrated the presence of fibronectin and fibromodulin in tECM, HYAL-tECM, and ChABC-tECM, but not in P-tECM (FIG. 42). Biglycan was detected in HYAL-tECM and ChABC-tECM but not tECM or P-tECM. The sGAG content in tECM was significantly higher than HYAL-tECM and ChABC-tECM (FIG. 43). Interestingly, a growth factor array (FIG. 44) revealed similar levels of FGF-2, EGF, IGF-1, and TGF-β3 in tECM, P-tECM, HYAL-tECM, and ChABC-tECM.

Each enzymatically treated tECM was then tested for its bioactivity by inclusion as a cell culture supplement, in comparison to supplementation with 2% FBS, Coll, and tECM (FIG. 41). hASCs were isolated from the infrapatellar fat pad tissues of patients (4 donors, 59-74 years old, male and female) undergoing total knee replacement surgery. Cells were sorted by flow cytometry with a mesenchymal stem cell (MSC) isolation kit (BD Bioscience, USA; positive markers CD90, CD105, CD73, and CD44 and negative markers CD34, CD116, CD19, CD45, and HLA-DR) and suspended in alpha-minimum essential medium (a-MEM; Gibco, USA) containing 10% (v/v) fetal bovine serum (FBS; Gibco), 1% (v/v) penicillin/streptomycin (P/S; Gibco), and 1 ng/mL fibroblast growth factor-2 (FGF-2; R&D Systems, USA). Rigorous quality control procedures were conducted to minimize any variability in cell behavior caused by different FBS batches. For example, clonal proliferation was tested by colony formation unit fibroblast assay (CFU-F), and differentiation multipotency was verified using a trilineage differentiation assay (osteogenic, adipogenic, and chondrogenic) at early passages (before 3) and late passages (after 8). All experiments with hASCs were performed at passages 3-5.

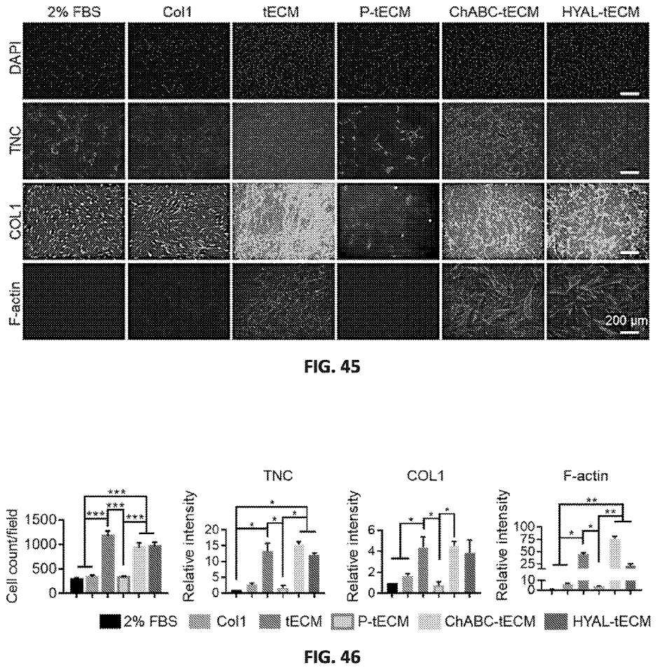

Cell proliferation was estimated using DAPI-based cell counting, and tenogenic differentiation was assessed based on presence of tenogenic markers (TNC, COL1) detected by immunofluorescence staining and phalloidin-stained cytoskeletal F-actin (FIGS. 45 and 46). Briefly, at each time point, the cells were fixed with either ice-cold methanol at −20° C. or 4% (w/v) paraformaldehyde (PFA; Sigma-Aldrich) at room temperature for 20 minutes. For the PFA-fixed samples, the cells were permeabilized with saponin solution (0.5% (w/v) saponin in PBS; Sigma-Aldrich) at room temperature for 20 minutes. The sample was incubated with 10% (v/v) donkey serum (Sigma-Aldrich) to block non-specific bonding. Antibodies were diluted accordingly with 0.1% (w/v) saponin and 1% (w/v) bovine serum albumin (BSA; Thermo Fisher Scientific) in PBS. The sample was incubated with the primary antibody overnight at 4° C. and with the secondary antibody for 1 hour at room temperature. The following antibodies were used: mouse monoclonal anti-tenascin C (TNC, Abeam, UK; 1:500), rabbit monoclonal anti-collagen type I (COL1, Novus Biologicals; 1:200), and mouse monoclonal anti-seleaxis (SCX; Abeam; 1:500). Alexa Fluor 488- and 647-conjugated antibodies (Abeam; 1:500, respectively) were used as secondary antibodies. Cell nuclei were identified by DAPI (Abeam; 1:6000), and F-actin was identified by Alexa Fluor 555-conjugated phalloidin (Thermo Fisher Scientific; 1:1000).

DAPI-stained images and DAPI cell counting suggested that the tECM-, ChABC-tECM-, and HYAL-tECM-treated groups exhibited significantly higher proliferation, while the result in the P-tECM-treated group was similar to that in the 2% FBS- and Coll-treated groups (FIGS. 45 and 46). Semi-quantitative analysis of fluorescence signal showed that the staining intensities of TNC, COL1, and F-actin were comparable among the tECM-, ChABC-tECM-, and HYAL-tECM-treated groups. On the hand, the staining intensities of these markers were largely decreased in the P-tECM-treated group compared to the tECM-treated group but similar to those in the 2% FBS- and Coll-treated groups (FIGS. 45 and 46).

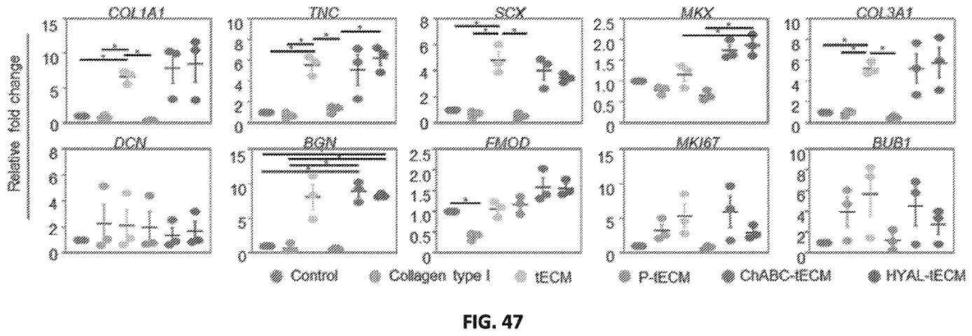

Additionally, to further characterize the pepsin digestion mediated reduction of tECM bioactivity, hASCs were cultured for 3 and 6 days followed by qPCR to assess the gene expression of tenogenic markers and proliferation-associated markers, i.e., SCX, COL1A1, TNC, MKX, COL3A1, DCN, BGN, FMOD, MKI67, and BUBI (FIG. 47). At each time point, total RNA was isolated with an RNA extraction kit (QIAgen, USA) and cDNA was synthesized with a reverse transcription PCR (RT-PCR) kit (New England Biolabs, USA) according to the manufacturer's protocols. qPCR was performed using TaqMan on a qPCR system (Applied Biosystem, Life Technology, USA), and the results were quantified by the comparative Ct method. A 6-FAM dye-labeled TaqMan MGF probe (Thermo Fisher Scientific) was used to identify tenogenesis marker genes, including scleraxis (SCX; Hs03054634_g1), collagen type I (COL1A1; Hs00164004_m1), tenascin-C(TNC; Hs01115665_m1), and mohawk (MKX; Hs00543190_ml). The expression level of each gene was normalized to a housekeeping gene (GAPDH; Hs02758991_g1). The analysis results revealed that hASCs treated with HYAL-tECM and ChABC-tECM exhibited comparable expression levels of tenogenesis-associated genes, including COL1A1, TNC, SCX, MKX, COL3A1, and BGN when compared to the tECM group. In contrast, compared to tECM, HYAL-tECM, and ChABC-tECM group, the P-tECM group displayed a significantly lower expression level of gene expression (i.e., COL1A1, TNC, SCX, MKX, COL3A1, and BGN), which was similar to the control and collagen type I groups (FIG. 47).

Taken together, these results demonstrated that tECM treatment enhanced hASC proliferation and tenogenic differentiation compared to cells cultured with 2% FBS or Coll. Pepsin treatment, which removed most of the non-collagenous components of tECM, significantly compromised its tenogenic activity. Other enzyme digestion groups did not show significant effects on the pro-tenogenesis activity of tECM.

### Example 9. Bioactivity of P-tECM Supplemented with Tenogenesis-Associated Growth Factors

As shown previously (G. Yang et al., Biomaterials 34, (2013): 9295), tECM extract contains various growth factors (GFs) that have been reported to regulate tenogenic differentiation during tendon development and repair, such as TGF-βs, IGF-1, and FGF-2 (T.

Molloy, Y. Wang & G. Murrell, Sports Med. 33, (2003): 381). The significant reduction of bioactivity in the acid-pepsin treated P-tECM preparation (FIGS. 40-47) suggests possible functional involvement of one or more of these GFs in the pro-tenogenesis effect of tECM due to degradation and/or inactivation of the GFs (A. J. Zollinger & M. L. Smith, Matrix Biol. 60-61, (2017): 27; T. A. H. Jarvinen & E. Ruoslahti, Br. J. Pharmacol. 176, (2019): 16). To test this hypothesis, the tenogenic bioactivity of P-tECM with or without supplementation of different GFs was investigated. The concentrations of GFs were set at a low dose (10 ng/mL) and a high dose (50 ng/mL) based on previous reports (G. Yang, B. B. Rothrauff, H. Lin, S. Yu & R. S. Tuan, Tissue Eng. Part A 23, (2017): 166; N. L. Leong et al., J. Orthop. Res. 38, (2020): 7).

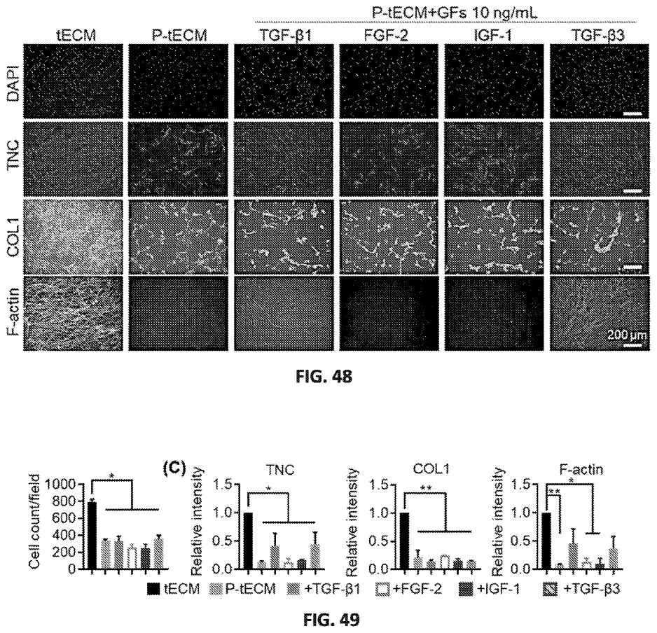

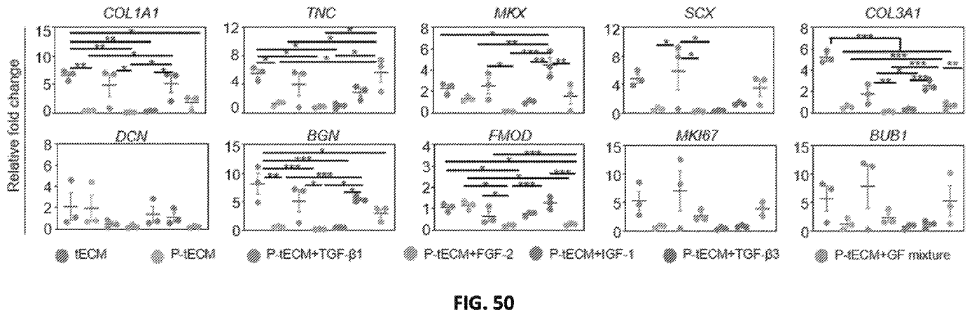

Cell counting based on DAPI nuclear staining showed that cell proliferation in P-tECM cultures supplemented with 10 ng/mL GF (i.e., TGF-β1, or FGF-2, or IGF-1, or TGF-β3) was not comparable to those exposed to the undigested tECM (FIGS. 48-50). The supplementation of TGF-β1 and the mixed GFs resulted in higher expression levels of proliferation-associated markers (MKI67 and BUB1), although these differences did not reach statistical significance when compared to the other groups (FIG. 50). Immuno- and cytoskeletal staining also revealed low staining intensities of TNC, COL1, and F-actin in all GF-supplemented groups (FIGS. 48 and 49). Specifically, COL1 in the tECM-treated group showed a fibrous structure, while discrete aggregation of COL1 was observed in the P-tECM-treated groups with or without 10 ng/mL GF supplementation (FIGS. 48 and 49). While the P-tECM-treated groups did show the presence of COL 1, they lacked the characteristic fibrous network arrangement observed in the tECM-treated group (FIGS. 48 and 49). Semi-quantitative analyses showed that the staining intensities of TNC and COL1 in all GF-supplemented P-tECM-treated groups were significantly lower than those in the tECM-treated group on day 6 (FIG. 49). The qPCR results showed that the treatment of TGF-β1 and TGF-β3 induced higher expression levels of tenogenesis-associated markers (i.e., COL1A1, TNC, MKX SCX, COL3AI, BGN, and FMOD) compared to FGF-2 and IGF-1 group. Interestingly, the gene expression levels of COL1A1, MKX, COL3A1, DGN, BGN, and FMOD in the group treated with a mixture of GFs was lower than those in the TGFs group (FIG. 50).

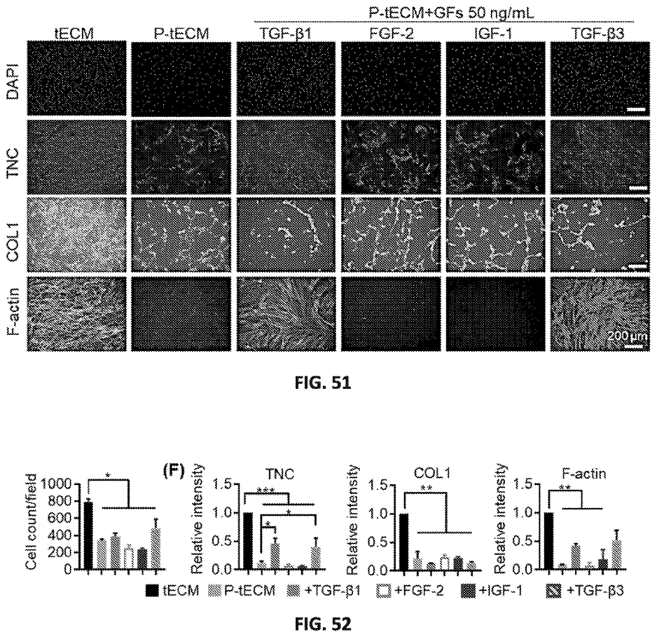

To assess whether the activities of the GFs were dose dependent, the effects of GF supplementation at 50 ng/mL were also examined. Interestingly, although TGF-β1, FGF-2, TGF-β3, or a mixture of GFs induced comparable expression levels of proliferation-associated genes (BGN and FMOD) as tECM, DAPI nuclear staining showed that cell proliferation in the high dose GF supplemented group was still not comparable to those exposed to the tECM (FIGS. 51-53). Both TGF-(31 and TGF-β3 enhanced the staining intensity of TNC, but not COL1 and F-actin, compared with that in the P-tECM-treated group but still not comparable to tECM-treated group (FIGS. 51-53). The staining intensities of these markers in the FGF-2- and IGF-1-supplemented P-tECM-treated groups remained reduced compared to the tECM-treated group (FIGS. 51-53). In contrast, qPCR analysis demonstrated that the application of TGF-β1, TGF-β3, and the GF mixture led to comparable expression levels of tenogenesis-associated genes, such as COL1A1, TNC, SCX, COL3A1, DCN, and BGN, in comparison to the tECM group (FIG. 53).

Taken together, these results showed that direct “add back” or supplementation with tenogenic GFs could not fully restore the lost bioactivity of acid-pepsin digested tECM, in terms of cell proliferation and tenogenic differentiation. These data thus suggest that, in addition to these known tenogenic GFs, other factors present in the non-collagenous ECM components also play an important role in tECM tenogenic bioactivity.

### Example 10. Fractionation of tECM into CM- and NCM-Enriched Fractions

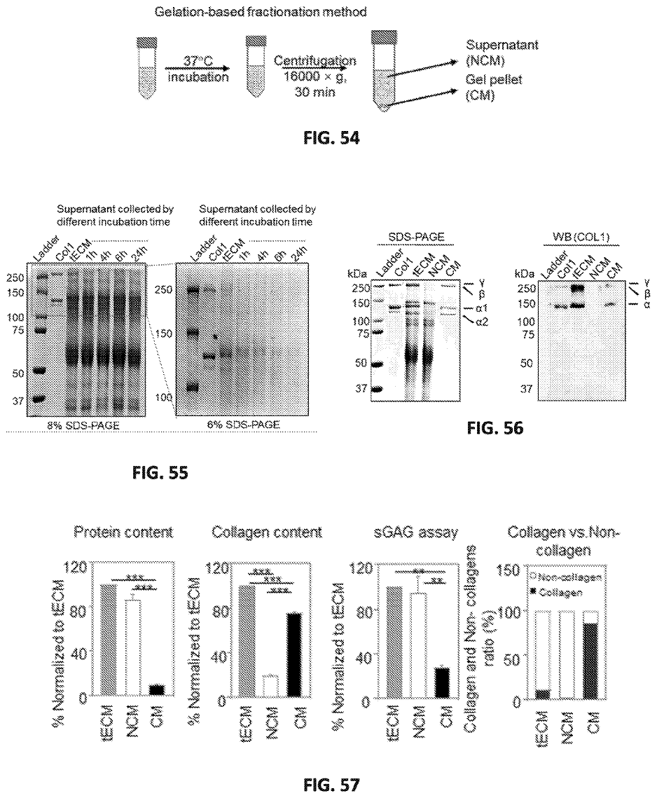

To assess the relative functional importance of collagenous versus non-collagenous components in the pro-tenogenesis bioactivity of tECM, tECM was fractionated into CM and NCM fractions their bioactivity was assessed on hASCs. A modified fractionation method based on the thermal gelation property of collagen was used (P. F. Slivkaet al., Biomater. Sci. 2, (2014): 1521). As shown in FIG. 54, tECM was incubated at 37° C. and later subjected to high-speed centrifugation, yielding a gel pellet containing insoluble CM components as well as a clear supernatant containing NCM components. More specifically, tECM was incubated in a 37° C. water bath to induce the polymerization of collagen. Subsequently, tECM was centrifuged at 16000×g for 30 minutes, and the supernatant was collected as the NCM fraction, while the precipitated pellet was collected as the CM fraction. The suspension of CM fraction was vigorously pipetted through a 10-μL pipet tip to homogenize the material as much as possible (P. F. Slivka et al., Biomater. Sci. 2, (2014): 1521). All the fractionated samples were resuspended to their original volume using PBS. Since the gelation-based fractionation method included a thermal incubation step, the incubation time was optimized for effective separation of the CM and NCM fractions and minimizing protein degradation that could influence tECM bioactivity. SDS-PAGE/Coomassie blue staining showed that collagens and non-collagens were separated only after incubation for 4 hours and longer (FIG. 55). Therefore, among four different thermal incubation durations (1, 4, 6, and 24 hours), the 4-hour incubation time was selected for subsequent experiments.

SDS-PAGE, Western blot (WB), BCA, and hydroxyproline assays were used to characterize the tECM fractionation outcome (FIGS. 56-58). For the WB assay, the gel was soaked in transfer buffer (Sigma-Aldrich) and l g/L SDS in dH₂O. The protein was transferred onto a PVDF membrane (Thermo Fisher Scientific) using a wet transfer system (Bio-Rad, USA), and incubated with a primary antibody (rabbit anti-human collagen type I, 1:1000; Novus Biologicals, USA) and a secondary antibody (goat anti-rabbit secondary antibody; 1:5000; Bio-Rad). The membrane was allowed to react with a chemiluminescence substrate (Thermo Fisher Scientific) and imaged immediately using a ChemiDoc imaging system (Bio-Rad). For the hydroxyproline assay, collagen concentration in different tECM fractions was estimated using a chloramine-T hydroxyproline assay (D. D. Cissell, J. M. Link, J. C. Hu & K. A. Athanasiou, Tissue Eng. Part C Methods 23, (2017): 243, standardized with commercial bovine collagen type I solution (Coll, Advanced BioMatrix, Inc., USA). All samples in triplicate were placed in a 96-well plate (200 μL/well) and A₅₅₀ values determined spectrophotometrically in a SpectraMax i3X Multimode Microplate Reader (Molecular Devices, USA).

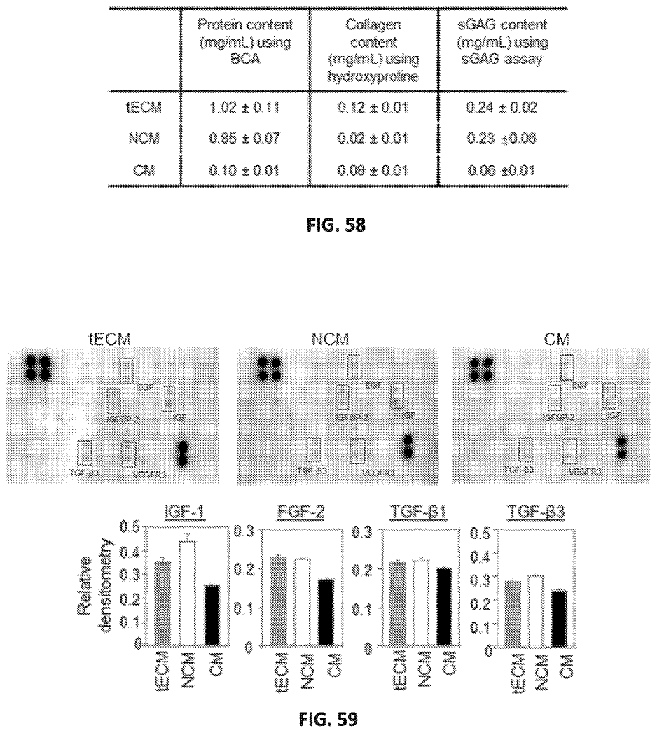

SDS-PAGE revealed the presence of collagen bands, i.e., monomeric α-, dimeric β-, and trimeric γ-chains, in tECM and the CM fraction but not in the NCM fraction, whereas non-collagenous components were mainly observed in tECM and the NCM fraction (FIG. 56). SDS-PAGE revealed the presence of collagen bands, i.e., monomeric α-, dimeric β- in the tECM and the CM fractions but not in the NCM fraction, whereas non-collagenous proteins were mainly observed in the tECM and the NCM fractions (FIG. 56). WB showed that the presumptive collagen bands in the CM fraction were positively immunostained with a COLI antibody (FIG. 56). It is important to note that the modified fractionation process, involving steps such as centrifugation, gelation, resolubilization, and separation, may potentially lead to some loss of collagen content in the CM. Therefore, BCA, hydroxyproline, and sGAG assays were conducted to accurately quantify the content. BCA assay showed that the final protein concentrations of the tECM, NCM fraction, and CM fraction were 1.02±0.11 mg/mL, 0.85±0.07 mg/mL (97.48%), and 0.1±0.001 mg/mL (9.92%), respectively (FIGS. 57 and 58). Additionally, a hydroxyproline assay was performed to quantify the collagen contents in the different fractions (C. M. da Silva, E. Spinelli & S. V. Rodrigues, Food Chem. 173, (2015): 619), revealing contents of 0.12±0.01 mg/mL, 0.02±0.01 mg/mL (14.710%), and 0.09±0.01 mg/mL (85.28%) in the tECM, NCM and CM fractions, respectively (FIGS. 57 and 58). Moreover, the sGAG assay showed that sGAG contents in the tECM, NCM and CM fractions were 0.24±0.02 mg/mL, 0.23±0.06 mg/mL, and 0.06±0.01 mg/mL respectively (FIGS. 57 and 58). Notably, tECM and NCM exhibited higher sGAG levels compared to CM, while CM exhibited significantly higher collagen levels compared to the other fractions (FIGS. 57 and 58). Interestingly, the GF array showed that tECM and NCM exhibited similar amount of FGF-2, EGF, IGF-1, and TGF-β3. Conversely, the GF content in the CM group was slightly lower than that in the other groups (FIG. 59).

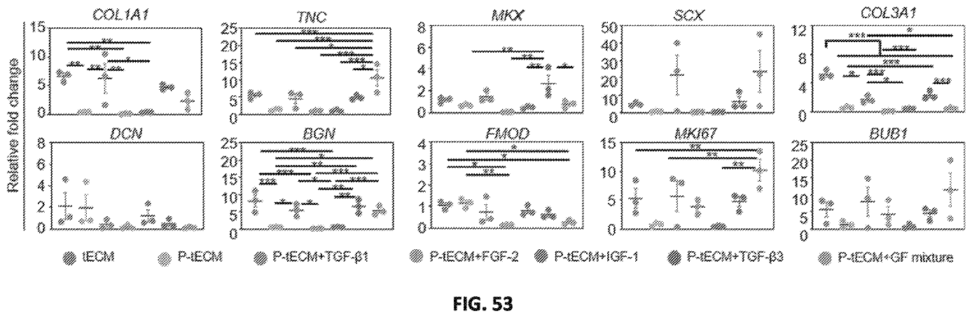

As compared to CM fraction of tECM, NCM fraction of tECM possessed a reduced amount of collagens as indicated by the hydroxyproline assay, but high amount of non-collagenous components as seen by SDS-PAGE (FIGS. 50-53). Notably, WB analysis revealed that collagen type I is the main component in CM fraction (FIG. 52). Therefore, the results suggest that the gelation-based, high-speed centrifugation method effectively fractionated tECM into NCM and CM fractions, which were subsequently tested for their pro-tenogenesis bioactivity on hASCs.

### Example 11. Pro-Tenogenesis Bioactivity of NCM and CM tECM Fractions in 2D Culture

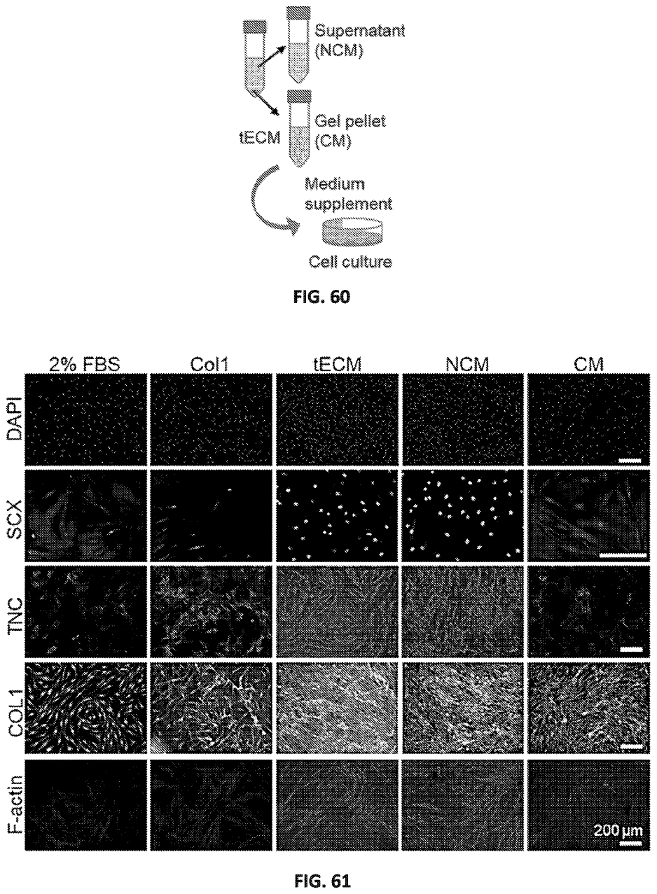

The pro-tenogenesis bioactivity of the NCM and CM fractions of tECM on 2D hASC cultures was tested, with 2% FBS and Coll as controls (FIG. 60). For 2D cell culture, hASCs (1×10⁴ cells/cm²) were seeded on tissue culture plates in Dulbecco's modified Eagle's medium (DMEM; Gibco) with 2% (v/v) FBS, 1% (v/v) insulin-transferrin-selenium-X (ITS-X; Gibco), 1% (v/v) P/S, 50 μg/mL ascorbic acid (Sigma-Aldrich), and 10% (v/v) of one of the following supplements: Coll (1 mg/mL, Advanced BioMatrix), tECM (1 mg/mL), pepsin digested tECM (P-tECM, 1 mg/mL), HYAL digested tECM (HYAL-tECM, 1 mg/mL), and ChABC digested tECM (ChABC-tECM, 1 mg/mL). Additionally, based on prior work, tECM (500 μg/mL) contains various GFs and GF-binding proteins, including TGF-β1 (872.2 pg/mL), TGF-β3 (34.6 pg/mL), insulin-like growth factor binding protein-3 (IGFBP-3, 162.8 pg/mL), and FGF-2 (469.4 pg/mL) (B. B. Rothrauff, G. Yang & R. S. Tuan, Stem Cell Res. Ther. 8, (2017): 133), which have established roles in tendon healing and regeneration (T. Molloy, Y. Wang & G. Murrell, Sports Med. 33, (2003): 381). Since pepsin treatment may either digest the GF within tECM or inactivate GF bioactivity (T. Marchbank, R. Boulton, H. Hansen & R. J. Playford, Gut 51, (2002): 787; V. Beachley et al., J. Biomed. Mater. Res. A 106, (2018): 147; R. J. Playford et al., Gastroenterology 108, (1995): 92), the effects of P-tECM supplemented with GFs including IGF-1, TGF-β1, TGF-β3, or FGF-2 (10 or 50 ng/mL) on hASC tenogenesis were further investigated (I. Rajpar & J. G. Barrett, J. Tissue Eng. 10, (2019): 2041731419848776). For gelation-based fractionated tECM bioactivity test, same culture condition was applied on hASCs with the following supplements: NCM (1 mg/mL), CM (1 mg/mL), and mixture of NCM and P-tECM (NCM+P-tECM, with volume ratio of 5:1).

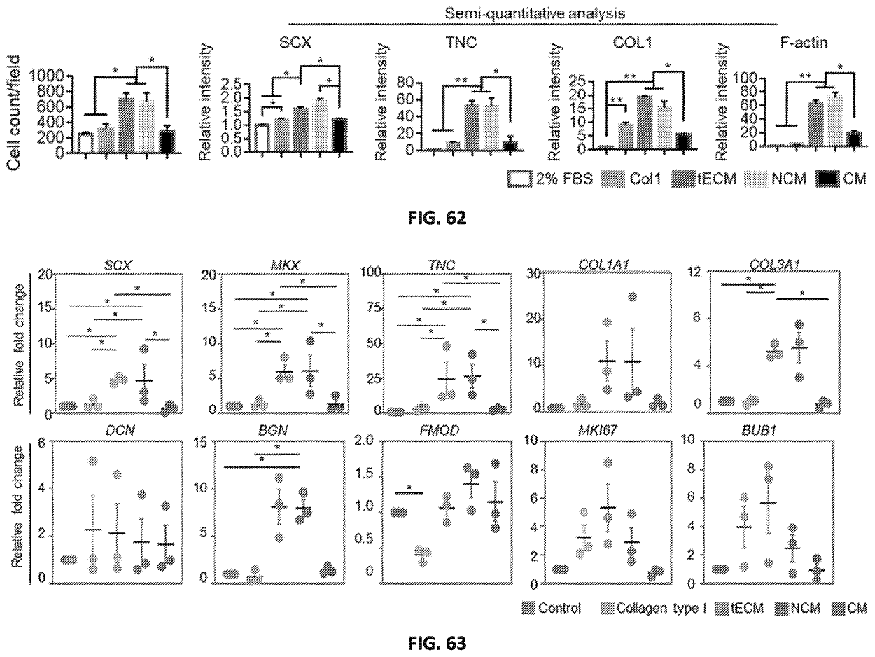

On culture day 6, DAPI-based cell counting and fluorescence staining were used to assess cell proliferation and tenogenic differentiation, respectively. DAPI-stained images and DAPI cell counting suggested that the cell proliferation in the NCM-treated group was comparable to that in the tECM-treated group and significantly higher than that in the 2% FBS-, Col1-, and CM-treated groups (FIGS. 61 and 62). Semi-quantitative analysis of immunostaining and cytoskeletal staining showed that tECM and its NCM fraction exhibited comparable fluorescence signal intensities of TNC, COL1, and F-actin, all of which were significantly higher than those in the other groups (FIGS. 61 and 62). Unlike the NCM-treated group, the CM-treated group showed significantly reduced cell proliferation and staining intensities of tenogenic markers compared to the tECM-treated group (FIGS. 61 and 62). Similarly, after 6 days of culture, qPCR assay showed that NCM-treated group exhibited increased gene expression of tenogenic markers (SCX, MKX, and TNC), which was comparable to the tECM-treated group and significantly higher than the 2% FBS-, Coll-, and CM-treated groups (FIG. 57).

Based on the results in FIGS. 45-48, direct “add back” or supplementation with tenogenic GFs could not fully restore the lost bioactivity of acid-pepsin digested tECM. Additionally, as the data from this Example (FIGS. 54-57) demonstrated that NCM exhibited tECM-like tenogenic bioactivity on hASCs, the bioactivity of P-tECM supplemented with NCM was further investigated (FIG. 58). On culture day 6, DAPI-stained images and DAPI cell counting suggested that the cell proliferation in the P-tECM+NCM-treated group was comparable to that in the tECM- and NCM-treated groups and significantly higher than that in the 2% FBS- and P-tECM-treated groups (FIGS. 59 and 60). Data from qPCR assays showed no significant difference of proliferation-associated gene expression (MKI67 and BUB1) among all the groups, while results from DAPI-stained images and DAPI cell counting indicated that the cell population in the NCM-treated group was comparable to that in the tECM-treated group and significantly higher than that in the control, collagen type I-, and CM-treated groups (FIG. 63). Furthermore, qPCR assays showed that the NCM-treated group showed increased gene expression of tenogenic markers (SCX, MKX, TNC, and BGN), which was comparable to the tECM-treated group and significantly higher than the control, collagen type I-, and CM-treated groups (FIG. 63).

Supplementation of tenogenic GFs (by directly “adding back” to the culture medium) was ineffective in restoring the lost bioactivity of acid-pepsin-digested tECM (FIGS. 48-53).

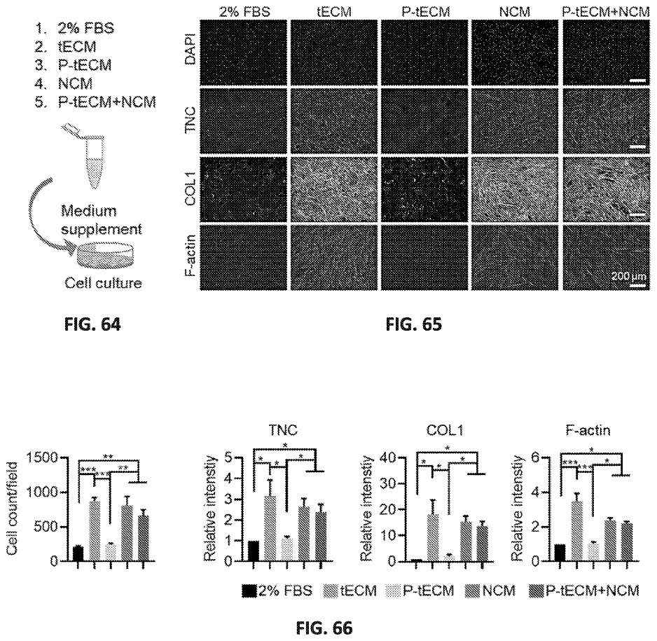

Additionally, as data (FIGS. 60-63) demonstrated that NCM exhibited tECM-like tenogenic bioactivity on hASCs, the bioactivity of P-tECM supplemented with NCM was also investigated (FIG. 64). Notably, the treatment of tECM with pepsin effectively digested most of the non-collagenous components under acidic condition (pH<2), as evidenced by SDS-PAGE gel (FIG. 40). It is noted that pepsin does not exhibit enzymatic activity on NCM under neutral conditions (pH=7). NCM (1 mg/mL) and P-tECM (1 mg/mL) were combined in a volumetric ratio of 5:1, resulting in a mixture with a composition of approximately 0.8 mg/mL of NCM and 0.2 mg/mL CM. This ratio closely approximates the relative proportions of CM and NCM found in tECM, as determined based on the results obtained from hydroxyproline assay and BCA assay (FIGS. 57 and 58).

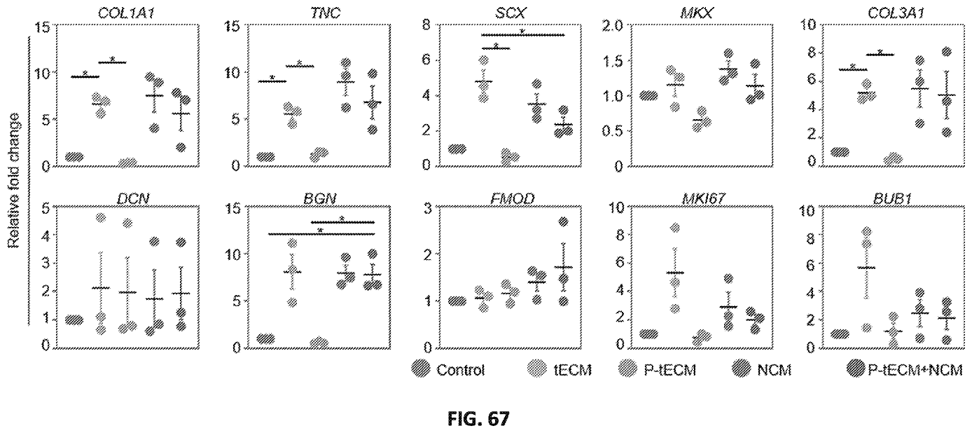

On day 6, although no significant difference of proliferation-associated gene expression was observed among all the groups, DAPI-stained images and DAPI cell counting suggested that the cell population in the P-tECM+NCM-treated group was comparable to that in the tECM- and NCM-treated groups and significantly higher than that in the control and P-tECM-treated groups (FIG. 5B). Semi-quantitative analysis of immunostaining and cytoskeletal staining showed that the NCM+P-tECM-treated group exhibited comparable fluorescence signal intensities of TNC, COL1, and F-actin in the tECM- and NCM-treated groups, which were significantly higher than those in the other groups (FIGS. 65 and 66). qPCR analysis showed comparable expression of tenogenesis-associated genes (i.e., COL1A1, TNC, SCX, COL3A1, and BGN) among tECM, NCM, and NCM+P-tECM groups. NCM+P-tECM showed significantly higher BGN expression than the control and P-tECM groups, while tECM showed significantly higher expression levels of COL1A1, TNC, and COL3A1 than the control and P-tECM groups (FIG. 67).

Taken together, the retention of pro-tenogenesis bioactivity in the NCM fraction strongly suggested that the non-collagenous components of tECM were primarily responsible for its tenogenic bioactivity on hASCs.

### Example 12. Transcriptomic Profiling of tECM-, CM-, and NCM-Induced hASC Differentiation

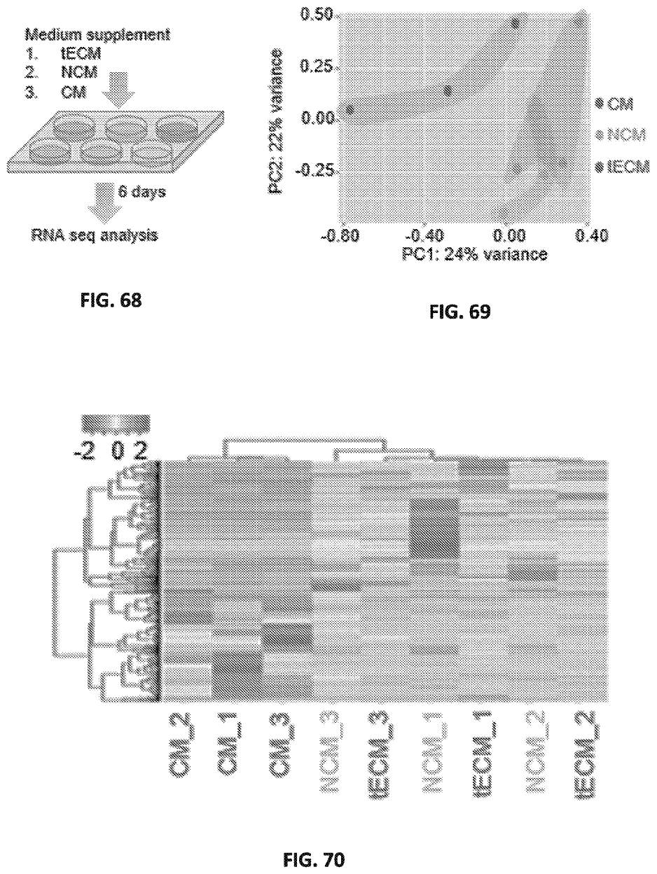

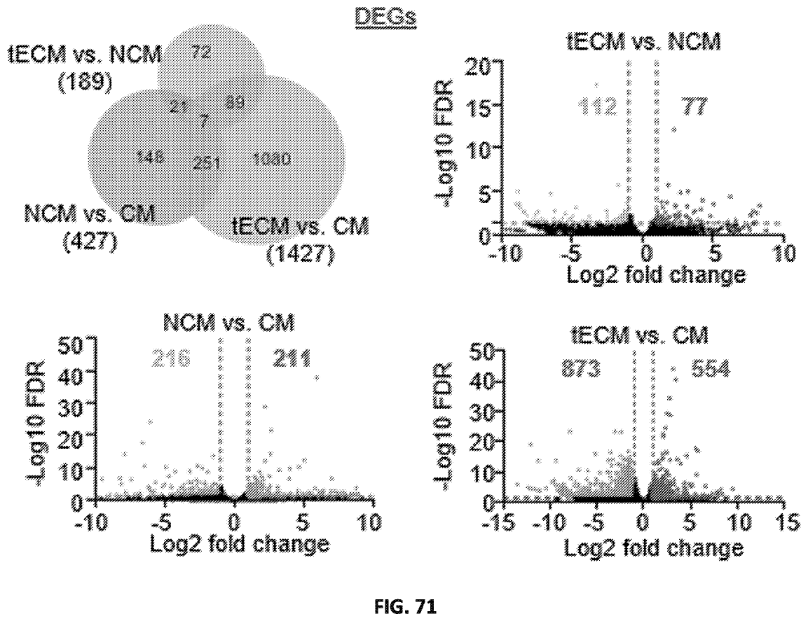

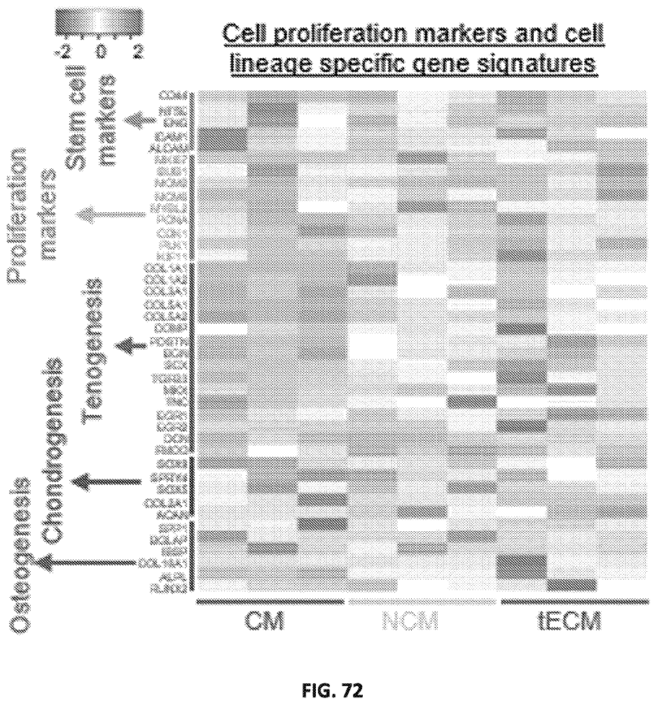

To assess the molecular events underlying tECM-, CM-, and NCM-driven pro-tenogenic differentiation of hASCs, total cellular RNA was extracted from hASCs cultured under three different conditions (tECM (1 mg/mL, 10% v/v), CM (1 mg/mL, 10% v/v), or NCM (1 mg/mL, 10% v/v)) for RNA-Seq analysis (FIG. 68). Principle component analysis (PCA) showed a clear segregation of different culture conditions, with the greatest variance on PC1 separating the CM group from the NCM and tECM groups (FIG. 69), which is consistent with the heatmap analysis of differentially expressed genes (DEGs) (FDR <0.05 with an absolute fold change of 2 or greater between comparisons) presented in FIG. 70. DEGs and the top-ranked DEGs including 5 upregulated (SPARC, HAPLN1, COL5AI, COL5A2, LRRC15) and 5 downregulated genes (TMEM35A, ABI3BP, NPTX1, PPL, HLA-DOA) are shown in FIG. 71. To characterize stem cell lineage commitment under different culture conditions, expression of established markers related to stem cells, proliferation, tenogenesis, chondrogenesis, and osteogenesis was examined. A heatmap of cell-specific gene signatures showed that both the tECM and NCM groups induced elevated expression of proliferation and tenogenesis genes compared with the CM group. In contrast, stem cell surface markers and chondrogenesis-related markers showed a higher expression in the CM group compared to tECM and NCM groups (FIG. 72).

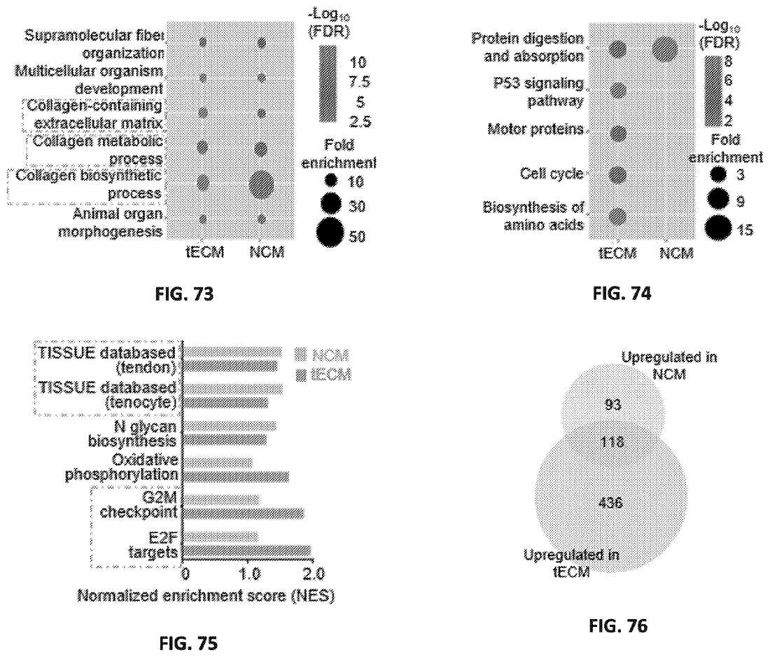

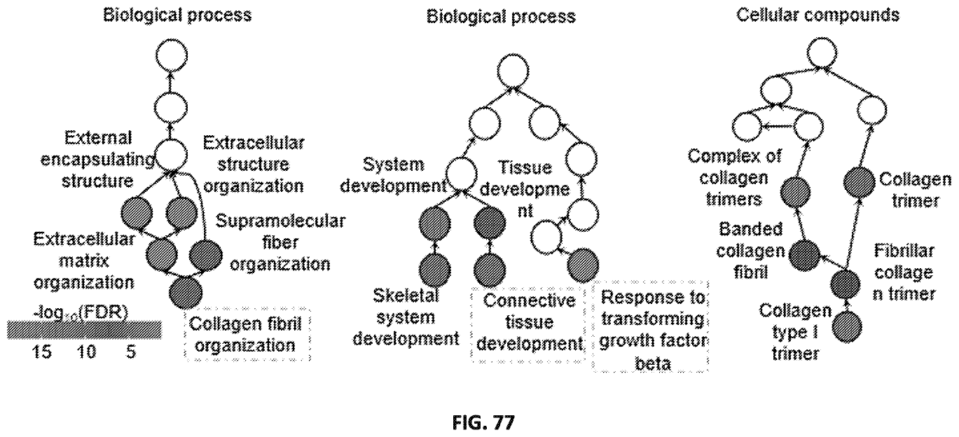

The similarities and differences in the transcriptomic profiles induced by the tECM and NCM groups in comparison to the CM group were next explored. Specifically, 1427 DEGs were found in the tECM group compared to the CM group, with 554 up-regulated and 873 down-regulated. When the NCM group was compared to the CM group, 427 DEGs were identified, with 211 up-regulated and 216 down-regulated (FIG. 71). Gene ontology (GO) enrichment analysis, KEGG pathway analysis, and gene set enrichment analysis (GSEA) were then performed on the DEGs between tECM vs. CM and NCM vs. CM. GO analysis revealed that compared to the CM group, the tECM and NCM groups had similar GO enrichment patterns, such as enhanced collagen synthesis (FIG. 73). KEGG Pathway and GSEA revealed that the “proliferation-related”, “tenocyte,” and “tendon” gene sets from the MSigDB hallmark gene sets, KEGG databases, and TISSUE gene sets were positively enriched (higher NES) in the tECM and NCM groups compared to the CM group (FIGS. 74 and 75). An additional comparison was made between the list of upregulated DEGs in both the tECM and NCM groups and the CM group, which included 118 genes in total. Enriched GO terms for these 118 genes included collagen fibril organization, connective tissue development, and fibrillar collagen trimer (FIGS. 76 and 77), and KEGG pathway analysis showed that these genes were involved in pathways related to focal adhesion and ECM-receptor interaction. Similarly, STRING analysis resulted in a dense network of proteins with three highly connected clusters centered around fibrillar collagen, collagen biosynthesis, and connective tissue. Moreover, the differences in the transcriptomic profiles induced by the tECM and NCM groups were observed by classifying the gene expression profiles between the two groups. Briefly, summarized from GO enrichment, KEGG pathway, STRING network, and GSEA analyses, compared to the NCM treatment, the tECM treatment was associated with GO terms or pathways related to amino acid-related pathways and cell cycle progression, which are involved in many essential biological processes, e.g., cell proliferation and collagen synthesis.

Taken together, this data highlights the distinct gene expression profiles of the tECM and NCM groups compared to the CM group, and demonstrates that the difference in gene expression between the tECM and NCM groups is minor. Importantly, tECM and NCM share similar GO enrichment patterns and pathways related to collagen synthesis; and induce enhanced proliferation and tenogenic differentiation (GSEA analysis and heatmap analysis of lineage-specific gene expression).

### Example 13. Pro-Tenogenesis Bioactivity of NCM and CM tECM Fractions in 3D Culture

The soluble ECM shows promise for use as an injectable solution or in conjunction with a wide range of diverse water-soluble biomaterials for various clinical applications. Therefore, the solubility of ECM emerges as a critical factor in these scenarios. Notably, when NCM and tECM were concentrated at 5 mg/mL, protein precipitation was observed in the tECM group but not in the NCM group, suggesting that NCM exhibited superior solubility than tECM at higher concentrations (FIG. 81).

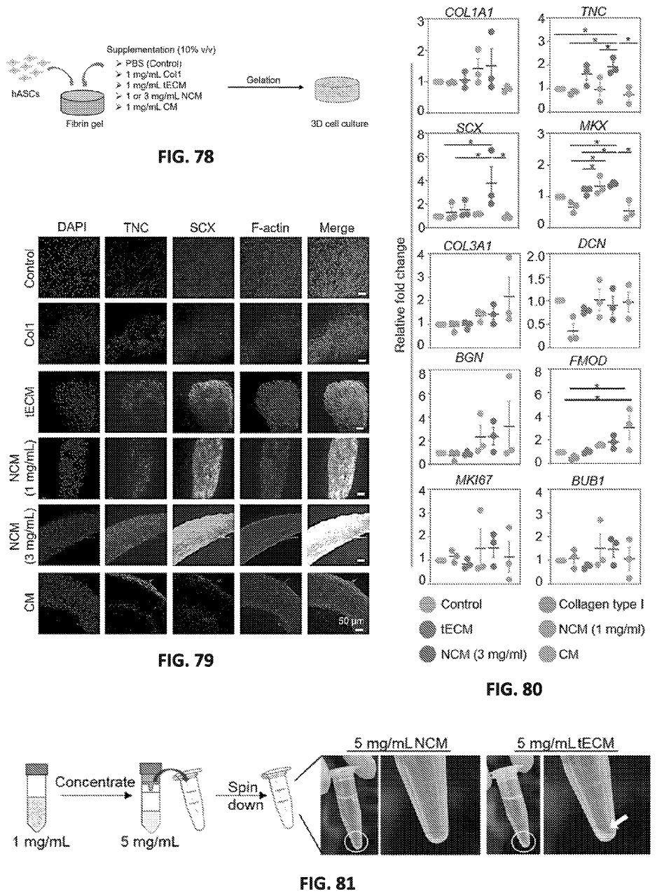

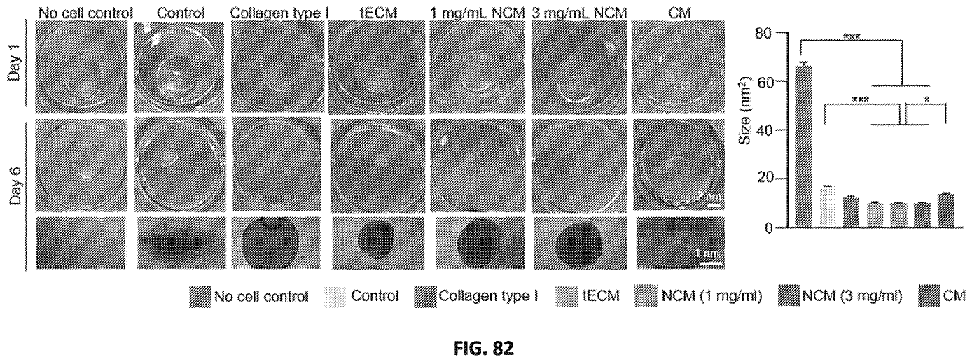

Based on data indicating the superior solubility and bioactivity (2D setting) of NCM fraction (FIG. 81), an additional example explored the tenogenesis bioactivity of the NCM fraction in 3D culture (FIG. 78). Coll (1 mg/mL), tECM (1 mg/mL), NCM (1 mg/mL and 3 mg/mL), and CM (1 mg/mL) were separately mixed with hASCs seeded in fibrin gels in 3D cultures, with fibrin gel only as a control group. To evaluate the morphological changes, the size of the gels was measured for up to 7 days. Interestingly, the results showed that hydrogels supplemented with tECM and NCM (1 mg/mL and 3 mg/mL) were significantly smaller than the other groups (FIG. 82). The release test revealed that the NCM (3 mg/mL) group exhibited a burst release of proteins within the first 2 days, which was significantly higher compared to other groups. Furthermore, the pro-tenogenesis effect was assayed by fluorescence staining and qPCR after 6 days of culture (FIGS. 79 and 80). For 3D cell culture, hASCs (2x 10⁶ cells/mL) were encapsulated in fibrin gels containing tECM fractions or Coll. Briefly, cells were suspended in fibrinogen (Enzyme Research Laboratories, USA) and thrombin (Enzyme Research Laboratories) before incubation for 10 minutes at 37° C. to induce gelation. The final concentration was 5 mg/mL fibrinogen, 1.5 U/mL thrombin, 50 ng/mL ascorbic acid, as well as 20% (v/v) of 1 mg/mL tECM, or 1 mg/mL Coll, or NCM (1 or 3 mg/mL) in each 300 μL fibrin gel (a round disk, 10 mm (diameter)×4 mm (thickness)). The hASC-encapsulated fibrin gels were cultured in DMEM containing 10% (v/v) FBS, 1% (v/v) P/S, and 50 μg/mL ascorbic acid for designated time points.

The tECM-, NCM (1 mg/mL and 3 mg/mL)-supplemented fibrin constructs showed enhanced staining intensity of SCX and TNC compared to those in the other groups (FIG. 62). Interestingly, hydrogel contraction was observed in tECM-supplemented and NCM-supplemented fibrin gels (FIG. 79). The qPCR results showed that NCM groups, both 1 mg/mL and 3 mg/mL, exhibited significantly enhanced expression levels of MKX over fibrin gel control group (FIG. 63). The 3-mg/mL NCM group showed significantly enhanced expression levels of TNC over the 1-mg/mL NCM group, and enhanced levels of SCX, MKX, and TNC over control, Coll-, and CM-treated groups. On the other hand, the CM group showed enhanced expression levels of FMOD compared with the collagen type I and control groups (FIG. 80).

Collectively, these results demonstrate that the NCM fraction displays superior solubility compared to tECM. Furthermore, when NCM is incorporated into a fibrin gel, it effectively stimulates hASC tenogenesis.

Although the foregoing disclosure has been described in some detail by way of illustration and example for purpose of clarity of understanding, one of skill in the art will appreciate that certain changes and modifications within the spirit and scope of the disclosure may be practiced, e.g., within the scope of the appended claims. It should also be understood that aspects of the disclosure and portions of various recited embodiments and features can be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only and is not intended to limit the disclosure. In addition, each reference provided herein is incorporated by reference in its entirety for all purposes to the same extent as if each reference was individually incorporated by reference.

What is claimed is:

1. A graft material comprising:
a shell comprising a photocrosslinkable hydrogel and an extracellular matrix; and
a core comprising an elastomeric polymer covalently crosslinked to the shell.

2. The graft material of claim 1, wherein the extracellular matrix comprises a tissue-derived extracellular matrix.

3. The graft material of claim 1 or 2, wherein the extracellular matrix comprises a cell-derived extracellular matrix.

4. The graft material of any one of claims 1-3, wherein the extracellular matrix is supplemented with a growth factor.

5. The graft material of any one of claims 1-4, wherein the photocrosslinkable hydrogel comprises the extracellular matrix.

6. The graft material of claim 5, wherein the concentration of the extracellular matrix in the photocrosslinkable hydrogel is between about 0.2 mg/mL and about 10 mg/mL.

7. The graft material of any one of claims 1-5, wherein the extracellular matrix is coated on a surface of the core.

8. The graft material of any one of claims 1-7, wherein the extracellular matrix comprises a urea-extracted extracellular matrix, or a non-collagenous matrix enriched fraction thereof.

9. The graft material of any one of claims 1-8, wherein the extracellular matrix comprises a tendon extracellular matrix.

10. The graft material of any one of claims 1-9, wherein the elastomeric polymer comprises a reaction product of a polyol, a polyisocyanate, and an acrylate.

11. The graft material of claim 10, wherein the polyol is an oven-dried polyol.

12. The graft material of any one of claims 1-11, wherein the elastomeric polymer comprises a reaction product of N,N,N′,N′-Tetrakis(2-hydroxypropyl)ethylenediamine, hexamethylene diisocyanate, and methacrylic anhydride.

13. The graft material of any one of claims 1-12, wherein the extracellular matrix comprises fibronectin, decorin, biglycan, fibromodulin, keratocan, prolargin, cartilage oligomeric matrix protein, thrombospondin 1, thrombospondin 4, TGF-β1, TGF-β3, IGF-1, FGF-2, or a combination thereof.

14. The graft material of any one of claims 1-13, wherein the photocrosslinkable hydrogel comprises gelatin or a derivative thereof.

15. The graft material of any one of claims 1-14, wherein the elastomeric polymer is crosslinked to the shell by a photocrosslinking agent, and wherein the photocrosslinking agent comprises benzophenone.

16. A method of repairing a defect of a tendon of a subject, the method comprising implanting the graft material of any one of claims 1-15 in the subject proximate to the defect of the tendon.

17. A method of producing a graft material, the method comprising:
providing an extracellular matrix;
forming a pre-hydrogel mixture comprising a photocrosslinkable polymer and the extracellular matrix;
providing an elastomeric polymer;
absorbing a photocrosslinking agent onto a surface of the elastomeric polymer, thereby yielding a treated elastomeric polymer;
applying the pre-hydrogel mixture to the treated elastomeric polymer, thereby creating a pre-graft material; and
irradiating the pre-graft material with ultraviolet light for an exposure duration, thereby producing the graft material.

18. The method of claim 17, wherein the photocrosslinking agent comprises benzophenone.

19. The method of claim 17 or 18, wherein the exposure duration is between 0.2 min and 10 min.

20. The method of any one of claims 17-19, wherein the providing of the extracellular matrix comprises:
incubating the extracellular matrix at a gelation temperature sufficient to promote gelation of a collagen matrix-enriched portion of the extracellular matrix; and
removing the collagen matrix-enriched portion from the extracellular matrix.

21. The method of any one of claims 17-20, wherein the providing of the elastomeric polymer comprises:
drying a polyol in an oven, thereby generating an oven-dried polyol;
forming a pre-elastomer mixture comprising the oven-dried polyol, a polyisocyanate, and an acrylate; and
irradiating the pre-elastomer mixture with ultraviolet light, thereby generating the elastomeric polymer.

22. The method of any one of claims 17-21, wherein the method further comprises:
seeding cells in or on the graft material.
